(19) United States
(12) Patent Application Publication
(10) Pub. No.: US 2008/0279874 A1
(43) Pub. Date: Nov. 13, 2008

(54) COMPOSITIONS AND METHODS FOR MODULATION OF PLK1 KINASE ACTIVITY

(75) Inventors: Frank Loganzo, JR., New City, NY (US); Girija Krishnamurthy, Chestnut Ridge, NY (US); Weidong Warren Ding, Orangeburg, NY (US); Xingzhi Cindy Tan, Congers, NY (US); Jagruti Hasmukh Patel, Saddle Brook, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/115,750

(22) Filed: May 6, 2008

Related U.S. Application Data
(60) Provisional application No. 60/974,618, filed on Sep. 24, 2007.
(60) Provisional application No. 60/916,433, filed on May 7, 2007.

(51) Int. Cl. A61K 39/00; C12N 9/12; G01N 33/573; G01N 33/53; G01N 33/569; G06G 7/58; A61P 37/04; A61K 38/16; G01N 33/567; C07K 14/00; C07K 16/40

(52) U.S. Cl. 424/185.1; 435/194; 435/7.4; 435/7.1; 435/7.37; 435/7.21; 530/324; 530/387.9; 514/12; 703/11

(57) ABSTRACT

Described are compositions and methods for activating a Plk1 protein as well as phospho-specific anti-Myt1 antibodies that can be used to detect phosphorylation of Myt1. Activated Plk1 protein, phospho-specific anti-Myt1 antibodies, and/or Plk1 substrates can be used in screening assays to identify compounds that modulate the ability of Plk1 to phosphorylate and/or bind to a Plk1 substrate.

### PRIOR APPLICATIONS

This Application claims priority to U.S. Application No. 60/916,433, filed May 7, 2007, and U.S. Application No. 60/974,618, filed Sep. 24, 2007. The entire content of these provisional applications is herein incorporated by reference.

### TECHNICAL FIELD

The invention relates to protein chemistry and cellular and molecular biology.

### BACKGROUND

Plk1 (polo-like kinase-1) is a serine/threonine kinase that has several critical functions in cell division, including initiation of mitosis, centrosome maturation, spindle assembly, anaphase regulation, and cytokinesis (Barr et al. (2004) Nat. Rev. Mol. Cell Biol. 5:429-440). However, Plk1 over-expression can increase cell proliferation and causes tumor growth in mouse models (Smith et al. (1997) Biochem. Biophys. Res. Commun. 234:397-405). In patients, Plk1 protein is over-expressed in solid tumors including those derived from colon, pancreas, prostate, ovary, endometrium, and skin (see, e.g., Takahashi et al. (2003) Cancer Sci. 94:148-152; Gray et al. (2004) Mol. Cancer Ther. 3:641-646; Weichert et al. (2004) Prostate 60:240-245; Takai et al. (2001) Cancer Lett. 164:41-49; Takai et al. (2001) Cancer Lett. 169:41-49; Kneisel et al. (2001) J. Cancer Res. Clin. Oncol. 127:S41; and Kneisel et al. (2002) J. Cutan. Pathol. 29:354-358). High levels of Plk1 mRNA have been reported in patient tumors derived from lung, head and neck, esophagus/stomach (Wolf et al. (1997) Oncogene 14:543-549; Knecht et al. (1999) Cancer Res. 59 2794-2797; and Tokumitsu et al. (1999) Int. J. Oncol. 15:687-692). High Plk1 has also been observed in samples derived from cases of lymphoma and non-Hodgkin's lymphoma (see, e.g., Ito et al. (2004) Anticancer Res. 24:259-263 and Mito et al. (2005) Leuk. Lymphoma 46:225-231). Some of these studies show a positive correlation between Plk1 expression in tumors and increased invasive potential in colorectal and endometrial carcinoma and reduced survival in patients with lung cancer, head and neck cancer, and non-Hodgkins lymphoma. Hence, inhibition of Plk1 may be useful for the treatment of a variety of cancers (Stebhardt and Ullrich (2006) Nat. Rev. Cancer 6:321-330).

### SUMMARY

The invention is based, at least in part, on the discovery that the kinase activity of a Plk1 protein can be enhanced by activation with ATP and a divalent cation such as manganese. The invention is also based, at least in part, on the discovery that an antibody that specifically recognizes a phosphorylated threonine 495 of membrane-associated tyrosine- and threonine-specific cdc-2 inhibitory kinase (Myt) (a phospho-specific anti-pT495 Myt1 antibody) can be used to detect phosphorylation of Myt1 by Plk1. Activated Plk1 protein and/or phospho-specific anti-pT495 Myt1 antibodies can be used, e.g., in screening assays to identify compounds that modulate (e.g., inhibit or stimulate) the ability of Plk1 to phosphorylate a Plk1 substrate. Such compounds (e.g., compounds that inhibit Plk1 activity) can be used in the treatment of cancers. Moreover, pre-activation of Plk1 kinase activity allows for use of the protein in ultra-high throughput screening assay formats, enabling simultaneous evaluation of large numbers of compounds for activity towards Plk1.

The invention is also based, in part, on the discovery that Plk1 interacts with and phosphorylates centromere protein B (CENPB). Accordingly, CENPB polypeptides can be used, for example, in screening assays to identify compounds that modulate (e.g., inhibit or stimulate) Plk1 kinase activity or compounds that modulate an interaction between Plk1 and CENPB. Compounds so identified can be used in the treatment of cancer. Moreover, CENPB polypeptides are useful as biomarkers to determine the efficacy of anti-Plk1 agents (e.g., in a subject to which an anti-Plk1 agent has been administered).

In one aspect, the disclosure features a method of activating a Plk1 protein by incubating a Plk1 protein in a buffer containing (i) a divalent cation selected from the group consisting of manganese, calcium, nickel, and zinc and (ii) ATP, wherein the divalent cation and ATP are present in amounts sufficient to increase the kinase activity of the Plk1 protein. The buffer can optionally contain MnCl₂ (e.g., at least 10 mM MnCl₂) and/or a detergent such as 3-[(3-Cholamidopropyl)dimethyl ammonio]-1-propanesulfonate (CHAPS; e.g., at least 0.05% CHAPS).

In some embodiments, at least 100 μg/ml (or at least 165 μg/ml) of the Plk1 protein can be incubated in the buffer. The Plk1 protein can be incubated in the buffer for a period of, e.g., at least one hour. The Plk1 protein can be activated either in the absence of a Plk1 substrate or in the presence of a Plk1 substrate (e.g., any of the Plk1 substrates described herein).

In another aspect, the disclosure features a method of detecting the kinase activity of a Plk1 protein, which method includes the following steps: providing a Plk1 protein activated by any of the methods described herein; contacting the activated Plk1 protein with a Plk1 substrate under conditions effective to permit phosphorylation of the Plk1 substrate; and measuring phosphorylation of the Plk1 substrate, wherein phosphorylation of the Plk1 substrate indicates kinase activity of the Plk1 protein.

The disclosure also features a method of detecting the kinase activity of a Plk1 protein, which method includes the following steps: contacting a Plk1 protein with a Plk1 substrate under conditions effective to permit phosphorylation of the Plk1 substrate, wherein the Plk1 substrate is a polypeptide containing full length Myt1 or a fragment of Myt1 that is subject to phosphorylation by Plk1 on a threonine residue that corresponds to position 495 of Myt1; contacting the Plk1 substrate with a phospho-specific anti-pT495 Myt1 antibody; and measuring binding of the antibody to the Plk1 substrate to thereby detect phosphorylation of the Plk1 substrate, wherein phosphorylation of the Plk1 substrate indicates kinase activity of the Plk1 protein. In some embodiments, prior to contacting the Plk1 protein with the Plk1 substrate, the Plk1 protein can be incubated in a buffer containing an amount of manganese and ATP sufficient to increase the kinase activity of the Plk1 protein.

In another aspect, the disclosure features a method of identifying a compound that inhibits phosphorylation of a Plk1 substrate, which method includes the steps of: providing a Plk1 protein activated by any of the methods described herein; contacting, in the presence of a candidate compound, the activated Plk1 protein with a Plk1 substrate; and measuring phosphorylation of the Plk1 substrate, wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

The disclosure also features a method of identifying a compound that inhibits phosphorylation of a Plk1 substrate, which method includes the following steps: contacting, in the presence of a candidate compound, a Plk1 protein with a Plk1 substrate, wherein the Plk1 substrate is a polypeptide containing full length Myt1 or a fragment of Myt1 that is subject to phosphorylation by Plk1 on a threonine residue that corresponds to position 495 of Myt1; contacting the Plk1 substrate with a phospho-specific anti-pT495 Myt1 antibody; and measuring binding of the antibody to the Plk1 substrate to thereby detect phosphorylation of the Plk1 substrate, wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

In some embodiments of the methods described herein, the contacting and/or the measuring can occur in a cell such as a mammalian cell (e.g., a human cell).

In some embodiments of the methods described herein, measuring phosphorylation of the Plk1 substrate can include the steps of: contacting the Plk1 substrate with an antibody that (i) is conjugated to a first fluorescent agent and (ii) specifically binds to the Plk1 substrate when the Plk1 substrate is phosphorylated on a serine or threonine residue, wherein the Plk1 substrate is conjugated to a second fluorescent agent; and detecting the occurrence of fluorescence resonance energy transfer between the first fluorescent agent and the second fluorescent agent as an indicator of phosphorylation of the Plk1 substrate.

In some embodiments of the methods described herein, measuring phosphorylation of the Plk1 substrate can include: contacting the Plk1 substrate with an antibody that (i) is conjugated to a detection moiety and (ii) specifically binds to the Plk1 substrate when the Plk1 substrate is phosphorylated on a serine or threonine residue; removing antibody that is not bound to the Plk1 substrate; and detecting the detection moiety associated with the Plk1 substrate as an indicator of phosphorylation of the Plk1 substrate.

In some embodiments of the methods described herein, measuring phosphorylation of the Plk1 substrate can include passaging the Plk1 substrate through a stationary phase, wherein increased or decreased retardation of the Plk1 substrate during passage through the stationary phase indicates the phosphorylation status of the Plk1 substrate.

The Plk1 substrate used in any of the methods described herein can optionally be, or contain, a polypeptide containing full length Myt1 or a fragment thereof that is subject to phosphorylation by Plk1. In some embodiments, the Plk1 substrate can be, or contain, a polypeptide containing a fragment of Myt1 that is subject to phosphorylation by Plk1 on a serine residue that corresponds to position 426 of Myt1, on a serine residue that corresponds to position 435 of Myt1, on a serine residue that corresponds to position 469 of Myt1, or on a threonine residue that corresponds to position 495 of Myt1. In some embodiments, the Plk1 substrate can contain, or consist of, the amino acid sequence as depicted in SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:14. The Plk1 substrate can be less than 50 amino acids in length.

The Plk1 substrate used in any of the methods described herein can optionally be, or contain, a polypeptide containing full length cell cycle phosphatase Cdc25C, Cyclin B1, early mitotic inhibitor 1 (Emi1), anaphase-promoting complex/cyclosome 1 (APC1), anaphase-promoting complex/cyclosome subunit 3 (APC3), anaphase-promoting complex subunit 8 (APC8), nucleolar phosphoprotein B23 (B23/Nucleophosmin), breast cancer type 2 susceptibility protein homolog (BRCA2), centrosomal protein of 55 kDa (Cep55), kinesin family member 23 (KIF23/CHO1/Mklp1), Cohesin, golgi reassembly stacking protein 1 (GRASP65), heat shock transcription factor 1 (HSF1), Kizuna, kinesin family member 20A (KIF20A/Mklp2/Rabkinesin6), Ndd1p , ninein-like protein (Nlp), nuclear migration protein nudC (NudC), p53, Plk1-interacting checkpoint helicase (PICH), peptidylprolyl cis/trans isomerase, NIMA-interacting 1 (Pin1), stathmin 1/oncoprotein 18 (Stathmin/Op18), translationally-controlled tumor protein homolog (TCTP), Vimentin, Wee1, centromere protein B (CENPB), tumor protein p73 (p73), Bora, DNA topoisomerase II alpha, origin recognition complex 1 (Hbo1), Aurora B, Mitotic centromere-associated kinesin (MCAK), Rho-associated, coiled-coil containing protein kinase 2, MLF1 interacting protein (PBIP1), budding uninhibited by benzimidazoles 1 homolog beta (BubR1), cytoplasmic polyadenylation element-binding protein (CPEB), human phosphatase HsCdc14A, small GTP/GDP-binding protein Ran or a fragment of any of these proteins that is subject to phosphorylation by Plk1.

In another aspect, the disclosure features a method of assessing the ability of a compound to inhibit phosphorylation of a Plk1 substrate by a Plk1 protein in a cell, which method includes the following steps: providing a cell expressing a Plk1 protein and a Plk1 substrate; incubating the cell in the presence of a compound identified by any of the methods described herein; and measuring the amount of the Plk1 substrate in the cell after incubating the cell in the presence of the compound, wherein a difference in the amount of the Plk1 substrate in the cell after incubation with the compound as compared to the amount of the Plk1 substrate in the cell in the absence of incubation with the compound indicates that the compound inhibits phosphorylation of the Plk1 substrate by the Plk1 protein. In one example of the practice of this method, the Plk1 substrate can be Emi1 and an increase in the amount of Emi1 in the cell after incubation with the compound as compared to the amount of Emi1 in the cell in the absence of incubation with the compound indicates that the compound inhibits phosphorylation of Emi1 by the Plk1 protein.

In another aspect, the disclosure provides an isolated peptide that is less than 50 amino acids in length and contains, or consists of, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or a variant thereof, wherein the variant is a phosphorylation substrate of Plk1. In some embodiments, the peptide contains, or consists of, a variant of SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12 in which at least one but not more than five amino acid residues are substituted, deleted, or inserted. In some embodiments, the amino acid sequence of the peptide is as depicted in SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

In some embodiments, a peptide described herein can be phosphorylated on a serine residue that corresponds to position 426 of Myt1, on a serine residue that corresponds to position 435 of Myt1, on a serine residue that corresponds to position 469 of Myt1, or on a threonine residue that corresponds to position 495 of Myt1. In some embodiments, the peptide contains, or consists of, the amino acid sequence depicted in SEQ ID NO:13.

In another aspect, the disclosure provides an isolated antibody that specifically binds to a peptide whose amino acid sequence consists of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13. In some embodiments, the antibody preferentially binds the peptide when phosphorylated on a threonine amino acid residue that corresponds to position 495 of Myt1.

In another aspect, the disclosure features a method of generating an immune response in a mammal by administering to the mammal an effective amount of any one of the peptides described herein.

In another aspect, the disclosure features a method of detecting the kinase activity of a Plk1 protein. The method includes the steps of: contacting a Plk1 protein with a Plk1 substrate under conditions effective to permit phosphorylation of the Plk1 substrate, wherein the Plk1 substrate is a polypeptide containing a CENPB protein or a fragment of a CENPB protein that is subject to phosphorylation by Plk1; and measuring phosphorylation of the Plk1 substrate, wherein phosphorylation of the Plk1 substrate indicates kinase activity of the Plk1 protein. The CENPB protein can contain, or consist of, the amino acid sequence of SEQ ID NO:19.

In some embodiments, the Plk1 substrate can be a polypeptide containing a fragment of a CENPB protein that is subject to phosphorylation by Plk1 on a serine residue that corresponds to position 43 of CENPB, on a serine residue that corresponds to position 156 of CENPB, on a threonine residue that corresponds to position 169 of CENPB, on a serine residue that corresponds to position 307 of CENPB, or on a threonine residue that corresponds to position 396 of CENPB. In some embodiments, the Plk1 substrate can contain, or consist of, SEQ ID NO:21, SEQ ID NO:22, SEQ ID NO:23, SEQ ID NO:24, or SEQ ID NO:25. In some embodiments, the Plk1 substrate can be less than 50 (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15,14, 13, 12, 11, 10, 9, or 8) amino acids in length.

In another aspect, the disclosure provides a method of identifying a compound that inhibits phosphorylation of a Plk1 substrate. The method includes the steps of: contacting, in the presence of a candidate compound, a Plk1 protein with a Plk1 substrate, wherein the Plk1 substrate is a polypeptide containing a CENPB protein or a fragment of a CENPB protein that is subject to phosphorylation by Plk1; and measuring phosphorylation of the Plk1 substrate, wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

In some embodiments of the above methods, measuring phosphorylation of the Plk1 substrate includes: contacting the Plk1 substrate with a phospho-specific anti-CENPB antibody; and measuring binding of the antibody to the Plk1 substrate to thereby detect phosphorylation of the Plk1 substrate. In some embodiments, the phospho-specific anti-CENPB protein antibody specifically recognizes CENPB at an epitope containing a phosphorylated serine residue that corresponds to position 43 of CENPB, a phosphorylated serine residue that corresponds to position 156 of CENPB, a phosphorylated threonine residue that corresponds to position 169 of CENPB, a phosphorylated serine residue that corresponds to position 307 of CENPB, or a phosphorylated threonine residue that corresponds to position 396 of CENPB.

In some embodiments of the above methods, measuring phosphorylation of the Plk1 substrate includes: contacting the Plk1 substrate with an antibody that (i) is conjugated to a detection moiety and (ii) specifically binds to the Plk1 substrate when the CENPB protein is phosphorylated on a serine or threonine residue; and detecting the detection moiety associated with the Plk1 substrate as an indicator of phosphorylation of the Plk1 substrate. The antibody can be, e.g., a phospho-specific anti-CENPB antibody that specifically recognizes CENPB at an epitope containing a phosphorylated serine residue that corresponds to position 43 of CENPB, a phosphorylated serine residue that corresponds to position 156 of CENPB, a phosphorylated threonine residue that corresponds to position 169 of CENPB, a phosphorylated serine residue that corresponds to position 307 of CENPB, or a phosphorylated threonine residue that corresponds to position 396 of CENPB.

In some embodiments of the above methods, measuring phosphorylation of the Plk1 substrate includes passaging the Plk1 substrate through a stationary phase, wherein an increased or decreased retardation of the Plk1 substrate during passage through the stationary phase indicates the phosphorylation status of the Plk1 substrate.

In another aspect, the disclosure features a method for identifying a compound that inhibits an interaction between a Plk1 protein and a Plk1 substrate. The method includes the steps of: contacting, in the presence of a candidate compound, a Plk1 protein with a Plk1 substrate, wherein the Plk1 substrate is a polypeptide containing a CENPB protein or a fragment of a CENPB protein that binds to Plk1; and measuring binding of the Plk1 protein to the Plk1 substrate, wherein decreased binding of the Plk1 protein to the Plk1 substrate in the presence of the candidate compound as compared to binding of the Plk1 protein to the Plk1 substrate that occurs in the absence of the candidate compound indicates that the candidate compound inhibits an interaction between the Plk1 protein and the Plk1 substrate.

In some embodiments of the above methods, the measuring can occur in a cell.

In another aspect, the disclosure features a method of inhibiting phosphorylation of a CENPB protein by a Plk1 protein by administering to a subject an effective amount of a compound that inhibits phosphorylation of a CENPB protein by a Plk1 protein. The disclosure also features a method of inhibiting an interaction between a Plk1 protein and a CENPB protein by administering to a subject an effective amount of a compound that inhibits an interaction between a Plk1 protein and a CENPB protein. The compound used in either of these methods can optionally be, or contain, a polypeptide containing a CENPB protein or a fragment of a CENPB protein that binds to Plk1. In some embodiments, the subject is a mammal such as a human. The subject can have, be suspected of having, or be at risk of developing, a cancer. In some embodiments, the above methods can include a step of determining if one or more cells of the subject's cancer express a Plk1 protein, a CENPB protein, or a Plk1 protein and a CENPB protein.

In another aspect, the disclosure features a method for evaluating the efficacy of an anti-Plk1 agent, which method includes the steps of: providing a biological sample obtained from a subject to whom an anti-Plk1 agent has been administered; and detecting phosphorylation of a CENPB protein in the biological sample, wherein a decreased level of phosphorylation of the CENPB protein as compared to the level of phosphorylation in a biological sample taken from another subject or from the subject prior to administration of the anti-Plk1 agent indicates that the anti-Plk1 therapy is effective.

In some embodiments, the anti-Plk1 agent inhibits Plk1 kinase activity and/or Plk1 expression. Inhibition of Plk1 expression can be, e.g., (i) inhibition of Plk1 protein expression; (ii) inhibition of Plk1 mRNA expression; or (iii) inhibition of Plk1 protein or Plk1 mRNA stability (e.g., increased degradation of a Plk1 protein or a Plk1 mRNA). Examples of anti-Plk1 agents include scytonemin, ON01910, and BI 2536.

In another aspect, the disclosure features an isolated peptide that is less than 50 (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length and contains a fragment of a CENPB protein that is subject to phosphorylation by Plk1 on a serine residue that corresponds to position 43 of CENPB, on a serine residue that corresponds to position 156 of CENPB, on a threonine residue that corresponds to position 169 of CENPB, on a serine residue that corresponds to position 307 of CENPB, or on a threonine residue that corresponds to position 396 of CENPB.

In another aspect, the disclosure features an isolated peptide that is less than 50 (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length and contains SEQ ID NO:21, SEQ ID NO:22, SEQ ID NO:23, SEQ ID NO:24, or SEQ ID NO:25, or a variant thereof, wherein the variant is a phosphorylation substrate of Plk1.

In some embodiments, the peptide contains, or consists of, SEQ ID NO:21, SEQ ID NO:22, SEQ ID NO:23, SEQ ID NO:24, or SEQ ID NO:25.

In some embodiments, the peptide contains, or consists of, a variant of SEQ ID NO:21, SEQ ID NO:22, SEQ ID NO:23, SEQ ID NO:24, or SEQ ID NO:25 in which at least one but not more than five amino acid residues are substituted, deleted, or inserted.

In some embodiments of the above peptides, the peptide is phosphorylated, e.g., on a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB.

In another aspect, the disclosure features an isolated antibody that specifically binds to any of the CENPB peptides described herein. In some embodiments, the antibody preferentially binds the peptide when phosphorylated on a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB.

In another aspect, the disclosure provides a method of generating an immune response in a mammal. The method includes the step of administering to the mammal an effective amount of any of the above peptides.

In another aspect, the disclosure features a method for generating a compound that inhibits the interaction between a Plk1 protein and a CENPB protein. The method includes the steps of: providing a three-dimensional structure of a molecule or a molecular complex containing (a) a Plk1 protein or a CENPB-binding fragment thereof, (b) a CENPB protein or a Plk1-binding fragment thereof, or (c) a molecular complex containing (a) and (b); designing, based on the three-dimensional structure, a compound containing a region that inhibits the interaction between a Plk1 protein and a CENPB protein; and producing the compound.

In another aspect, the disclosure features a compound generated by the above method and a pharmaceutical composition containing the above compound and a pharmaceutically acceptable carrier.

In another aspect, the disclosure features a composition (e.g., a protein array) containing at least two (e.g., at least three, at least four, at least five, at least six, at least seven, at least eight, at least nine, at least 10, at least 11, at least 12, at least 15, at least 20, at least 21, at least 22, at least 23, at least 24, at least 25, at least 30, or at least 35 or more) polypeptides or fragments thereof that are subject to phosphorylation by a Plk1 protein. The polypeptides include any of the Plk1 substrates described herein such as Myt1, Cdc25C, Cyclin B1, Emi1, APC1, APC3, APC8, B23/Nucleophosmin, CENPB, BRCA2, Cep55, CHO1/Mklp1, Cohesin, GRASP65, HSF1, Kizuna, Mklp2/Rabkinesin6, Ndd1p, Nlp, NudC, p53, Plk1-interacting checkpoint helicase (PICH), Pin1, Stathmin/Op18, TCTP, Vimentin, Wee1, tumor protein p73 (p73), Bora, DNA topoisomerase II alpha, origin recognition complex 1 (Hbo1), Aurora B, Mitotic centromere-associated kinesin (MCAK), Rho-associated, coiled-coil containing protein kinase 2, MLF1 interacting protein (PBIP1), budding uninhibited by benzimidazoles 1 homolog beta (BubR1), cytoplasmic polyadenylation element-binding protein (CPEB), human phosphatase HsCdc14A, or small GTP/GDP-binding protein Ran.

In some embodiments, the composition can contain one or more CENPB polypeptide and/or one or more Myt1 polypeptide. For example, the composition can include at least two of SEQ ID NOS: 2, or 4-12 and/or at least two of SEQ ID NOS:19, or 21-25. In some embodiments, the composition contains one or more CENPB polypeptide containing a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB. In some embodiments, the composition can contain a Myt1 polypeptide containing T495 of Myt1.

In some embodiments, the composition can contain a polypeptide containing one or more heterologous sequences.

In some embodiments, the composition contains less than 50,000 (e.g., less than 40,000, less than 30,000, less than 20,000, less than 15,000, less than 10,000, less than 5,000, less than 4,000, less than 3,000, less than 2,000, less than 1,500, less than 1,000, less than 750, less than 500, less than 200, less than 100, or less than 50) different polypeptides.

In some embodiments, the composition contains more than 50 (e.g., more than 60, more than 70, more than 80, more than 90, more than 100, more than 200, more than 500, more than 1000, more than 2000, more than 3000, more than 4000, more than 5000, more than 6000, more than 7000, more than 8000, or more than 10000 or more) different polypeptides.

In some embodiments of any of the compositions described above, the at least two polypeptides are bound to a solid support. The solid support can be a protein array chip, a particle (e.g., an encoded, magnetic, or magnetic and encoded particle), or any other solid support described herein.

In another aspect, the disclosure features a kit containing any of the compositions described above and optionally instructions for detecting the binding of a Plk1 protein to a polypeptide and/or instructions for detecting (or measuring) phosphorylation of a polypeptide by a Plk1 protein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE FIGURES

FIG. 1 is a line graph depicting the phosphorylation of Myt1_{—}7T peptide by recombinant Plk1 using a DELFIA® assay. The Y-axis represents the mean ± the standard deviation of signal emissions produced from europium (counts). The X-axis represents the concentration of ATP. The upper line (triangles) represents the data obtained for the phosphorylation of 1.0 μM biotinylated Myt1_{—}7T peptide and the lower line (squares) represents the data obtained for the phosphorylation of 0.5 μM biotinylated Myt1_{—}7T peptide.

FIG. 2 is a line graph depicting the effect of two different divalent cations on phosphorylation of Myt1_{—}13T peptide by recombinant Plk1 using the LANCE™ assay. The Y-axis represents the mean ± the standard deviation of APC/europium signal emissions at 665/615 nm. The X-axis represents the amount of input Plk1 in ng/well. The upper line (diamonds) represents the data obtained for the phosphorylation of biotinylated Myt1_{—b 13}T peptide at 10 mM MnCl₂. The middle line (squares) represents the data obtained for the phosphorylation of biotinylated Myt1_{—}13T peptide at 5 mM MnCl₂ and 5 mM MgCl₂. The lower line (triangles) represents the data obtained for the phosphorylation of biotinylated Myt1_{—}13T peptide at 10 mM MgCl₂.

FIG. 3 is a scatter plot graph depicting the results of a LANCE™ assay to determine the activity of pre-activated Plk1 and unactivated Plk1 towards Myt1_{—}13T peptide. The Y-axis represents the mean of APC/europium signal emissions at 665/615 nm. The X-axis represents the amount of input Plk1 in ng/well. The squares (upper) represent data points for pre-activated Plk1 and the diamonds (lower) represent the data points obtained for the unactivated Plk1.

FIG. 4 is a scatter plot depicting the results of a LANCE™ assay to determine the activity of Plk1 pre-activated with MnCl₂ with or without ATP. The Y-axis represents the mean signal to background ratio (S/B) of APC/europium signal emissions at 665/615 nm. The X-axis represents the amount of input Plk1 in nanograms (ng)/well. The diamonds (upper) represent data points for Plk1 pre-activated with MnCl₂ and ATP. The squares and the triangles (lower) represent the data points obtained for the unactivated Plk1 or Plk1 pre-activated with only MnCl₂ (no ATP).

FIG. 5 is a line graph depicting the results of a LANCE™ assay of a time course of pre-activation of Plk1. The Y-axis represents the mean signal to background ratio of APC/europium signals at 665/615 nm. The X-axis represents the amount of input Plk1 in ng/well. Plk1 was pre-activated at 4° C. for 1 hour (small rectangles), 2 hours (squares), 3 hours (diamonds), 4 hours (triangles), 6 hours (asterisks), or 20 hours (squares).

FIG. 6 is a scatter plot depicting the activity of Plk1 pre-activated with ATP and manganese or magnesium using the LANCE™ assay. The Y-axis represents the mean signal to background ratio (S/B) of APC/europium signal emissions at 665/615 nm. The X-axis represents the amount of input Plk1 innanograms (ng)/well. The diamonds (upper) represent data points for Plk1 pre-activated with MnCl₂ and ATP. The squares (lower) represent the data points obtained for the Plk1 pre-activated with MgCl₂ and ATP.

FIG. 7 is a line graph depicting activity of Plk1 pre-activated either at 165 μg/ml (2.36 μM) or in plate with a range of 0.006˜50 nM using the LANCE™ assay. The Y-axis represents the mean signal to background ratio (S/B) of APC/europium signal emissions at 665/615 nm. The X-axis represents the amount of pre-activated Plk1 (in nM) used in each LANCE™ assay reaction. The upper line (squares) represents the data obtained for the phosphorylation of biotinylated Myt1_{—}13T peptide by Plk1 pre-activated at 2.36 μM (165 μg/ml). The lower line (diamonds) represents the data obtained for the phosphorylation of biotinylated Myt1_{—}13T peptide by Plk1 pre-activated at a lower concentration (0.006˜50 nM).

FIG. 8 is a pair of photographs of an immunoblot and an autoradiograph depicting the auto-phosphorylation of PLK1 in the presence of manganese or magnesium. At room temperature for 0-3 hours, 165 μg/ml (2.36 μM) Plk1 was pre-activated with ATP (10 μM unlabeled ATP and 0.033 μM gamma-³³P labeled ATP) in the absence or presence of either 10 mM MnCl₂ or MgCl₂ and ATP. Following pre-activation, the reactions were subjected to SDS-PAGE and the gel was first stained with Coomassie blue and then dried. The top photograph represents dried SDS-PAGE gel depicting the migration profile of PLK1 relative to a molecular weight marker standard (lane 1). The lower photograph is an autoradiograph depicting autophosphorylated Plk1 (lanes 4 and 5).

FIG. 9A is a pair of photographs of immunoblots depicting the phosphorylation of a GST-Myt1 protein by Plk1. Recombinant human Plk1 kinase was incubated with GST-Myt1-B (0, 2.5, 5, 10 μg) in the presence or absence of wortmannin at 25, 50, or 100 nM, and without or with 100 μM ATP (∓). Samples were fractionated by sodium dodecyl sulfate polyacrylamide gel electrophoresis (SDS-PAGE) and immunoblotted with an antibody specific for phospho-Thr495-Myt1 (top) or an antibody specific for Myt1 protein (bottom).

FIG. 9B is a photograph of an immunoblot depicting inhibition of Plk1 phosphorylation of GST-Myt1 protein by wortmannin and BI2536. Recombinant human Plk1 kinase was incubated with 5 μg GST-Myt1-B in the presence or absence of 50 nM wortmannin or 0.1, 1, or 10 nM BI2536. Samples were fractionated using SDS-PAGE and immunoblotted with an antibody specific for phospho-Thr495-Myt1. “UT” refers to reactions in which no inhibitory compound was added.

FIG. 10A is a photograph of an immunoblot depicting the phosphorylation of Cdc25 protein by Plk1. 50 ng of recombinant human Plk1 kinase was incubated for 1 hour with full-length GST-Cdc25C (0, 0.1, or 1 μg) in the presence or absence of ATP (∓). The reaction samples were fractionated by SDS-PAGE and immunoblotted using an anti-phospho-Ser198-Cdc25C antibody.

FIG. 10B is a pair of photographs of immunoblots depicting the phosphorylation of Cdc25 protein by Plk1 in the presence or absence of wortmannin. 30 ng of recombinant human Plk1 kinase was incubated for 1 hour with 1 μg full-length GST-Cdc25C in the presence or absence of ATP (∓) and 0, 1, 10, or 100 nM wortmannin. The reaction samples were fractionated by SDS-PAGE and immunoblotted using anti-phospho-Ser198-Cdc25C (top photograph) or anti-Cdc25C antibodies (bottom photograph).

FIG. 11 is a photograph of an immunoblot depicting the phosphorylation of GST-Myt1-B protein using immunoprecipitated Plk1. Plk1 was immunoprecipitated from 1 mg of total protein lysate from HeLa cells grown in the absence (lane 1) or presence (lanes 2-4) of nocodazole. The immune complexes were subjected to Plk1 kinase assay using 5 μg of GST-Myt1-B protein. Reactions were performed in the absence (lane 2) or presence of either 50 nM wortmannin (lane 3) or 50 nM BI2536 (lane 4). Samples were fractionated by SDS-PAGE and immunoblotted with anti-P-Myt1-T495.

FIG. 12 is a line graph depicting the varying levels of protein kinase activity exhibited by different Plk1 proteins (LANCE™ assay). The Y-axis represents the signal to noise ratio (fluorescence signal from reactions containing ATP/fluorescence signal from reactions not containing ATP) and the X-axis represents the amount of Plk1 (ng/well) used in the reaction. The activities of wild type full length recombinant human Plk1 kinase (wt; diamonds), full length Plk1 with Thr 210 to Asp mutation (T210D; squares), and kinase-only domain (KD; triangles) were evaluated towards Myt_{—}13T using the LANCE™ assay.

FIG. 13A is a series of photographs of immunoblots depicting the effect of Plk1 knockdown on Myt1-T495 phosphorylation in mammalian cells. DU145 cells (left) and HeLa cells (right) were treated with or without nocodazole to induce mitotic arrest and increase Plk1 levels. Cells were incubated with either scrambled siRNA (scr; negative control) or Plk1 siRNA (Plki). Cells were lysed and proteins fractionated by SDS-PAGE. Immunoblotting was conducted sequentially for Plk1, phospho-T495-Myt1, and Myt1 protein. The molecular weights of the proteins given in units of kilodaltons (kD) are indicated at the right of each photograph.

FIG. 13B is a pair of photographs of immunoblots depicting the effect of Plk1 knockdown on Emil protein levels in mammalian cells. DU145 cells (left) were treated with or without nocodazole to induce mitotic arrest and increase Plk1 levels. Cells were incubated with either scrambled siRNA (scr; negative control) or Plk1 siRNA (Plki). Cells were lysed and proteins fractionated by SDS-PAGE. Immunoblotting was conducted sequentially for Plk1 and Emi1 protein. The molecular weights of the proteins given in units of kilodaltons (kD) are indicated at the left of each photograph.

FIG. 14 is a pair of photographs of immunoblots depicting the phosphorylation of CENPB by Plk1 in a solution-phase kinase assay. Kinase reactions were performed using combinations of recombinant Plk1 (50 ng), recombinant CENPB (1 μg), and ATP (100 μM) in the following kinase buffer (20 mM HEPES/10 mM MgCl₂/5 mM 2-glycerophosphate/0.5 mM L-cysteine). The reaction samples were fractionated by SDS-PAGE and immunoblotted using anti-phosphothreonine antibodies (top photograph) or anti-CENPB antibodies (bottom photograph). Molecular weights of the resolved proteins are in kDa and indicated to the left of each photograph.

FIG. 15 depicts the amino acid sequence for human CENPB polypeptide (SEQ ID NO:19) and indicates the position of amino acid residues phosphorylated in the presence of Plk1 (“Plk1”) as determined by mass spectrometry. Also included are amino acid residues whose phosphorylation is not dependent on Plk1 under the tested conditions (“basal”). Overlined residues indicate tryptic peptides identified as modified by phosphorylation. Underlined amino acid residues indicate residues, proximal to Plk1-dependent phosphorylation sites in CENPB that may also be phosphorylated by Plk1 but could not be unambiguously identified due to the nature of the analysis.

FIG. 16A is a series of photographs of immunoblots depicting the expression of His-epitope-tagged Plk1 (“His-PLK1”) and V5-epitope-tagged (“V5-CENPB”) in H1299 cells. Cells were transiently transfected with combinations of a plasmid encoding a His-PLK1 polypeptide and/or a plasmid encoding a V5-CENPB polypeptide. Following transfection and expression, cells were lysed and subjected to SDS-PAGE and immunoblotting using antibodies specific for CENPB (top photograph), Plk1 (middle photograph), or P-tubulin (lower photograph).

FIG. 16B is a pair of photographs of immunoblots depicting the interaction of Plk1 and CENPB in H1299 cells using immunoprecipitation. Cells transfected as described in FIG. 16A were lysed and subjected to immunoprecipitation using antibodies specific for Plk1. Immunoprecipitates were next subjected to SDS-PAGE and immunoblotting using antibodies specific for CENPB (top photograph) or Plk1 (lower photograph).

FIG. 17 is a series of photographs of immunoblots depicting increased phosphorylation of CENPB in H1299 cells expressing Plk1 and CENPB. Cells were transiently transfected with combinations of a plasmid encoding a His-PLK1 polypeptide and/or a plasmid encoding a V5-CENPB polypeptide. Following transfection and expression, cells were lysed and whole-cell extracts (WCE) subjected to SDS-PAGE and immunoblotting using antibodies specific for CENPB (top-most photograph; WCE), Plk1 (middle photograph; WCE), or β-tubulin (lower photograph; WCE). WCE were also subjected to immunoprecipitation (IP) using anti-V5 antibodies. Immunoprecipitates were subjected to SDS-PAGE and immunoblotting using anti-phosphothreonine antibodies (lower-most photograph).

FIG. 18, upper panel, is a photograph of an immunoblot depicting detection of inhibition of phosphorylation of threonine 495 of Myt1 by Plk1 kinase activity due to incubation of DU145 cells treated with nocodazole with BI2536.

FIG. 18, lower panel, is a photograph of an immunoblot depicting detection of Myt protein in DU145 cells treated with nocodazole and with BI2536.

FIG. 19 is a photograph of an immunoblot depicting stabilization of Emi1 levels in DU145 cells treated with nocodazole and incubated in the presence of BI2536.

### BRIEF DESCRIPTION OF THE SEQUENCE LISTING

SEQ ID NO:1 is the amino acid sequence of human Plk1 having GenBank® Reference AAA36659.

SEQ ID NO:2 is the amino acid sequence of human Myt1 having GenBank® Reference NP_{—}004194.

SEQ ID NO:3 is the amino acid sequence of human Cdc25C having GenBank® Reference NP_{—}001781.

SEQ ID NO:4 is the amino acid sequence of a fragment of Myt1 that can be used as Plk1 substrate.

SEQ ID NO:5 is the amino acid sequence of a fragment of Myt1 that can be used as Plk1 substrate.

SEQ ID NO:6 is the amino acid sequence of a fragment of Myt1 that can be used as Plk1 substrate.

SEQ ID NO:7 is the amino acid sequence of a fragment of Myt1 that can be used as Plk1 substrate.

SEQ ID NO:8 is the amino acid sequence of a fragment of Myt1 that can be used as Plk1 substrate.

SEQ ID NO:9 is the amino acid sequence of a fragment of Myt1 that can be used as Plk1 substrate.

SEQ ID NO:10 is the amino acid sequence of a fragment of Myt1 that can be used as Plk1 substrate.

SEQ ID NO:11 is the amino acid sequence of a fragment of Myt1 that can be used as Plk1 substrate.

SEQ ID NO:12 is the amino acid sequence of a fragment of Myt1 that can be used as Plk1 substrate.

SEQ ID NO:13 is the amino acid sequence of the phosphorylated peptide of SEQ ID NO:12.

SEQ ID NO:14 is the amino acid sequence of the human biologically active variant of Myt1, which has GenBank® Reference ID AAB71843.

SEQ ID NO:15 is the nucleotide sequence of p5, a Plk1-specific siRNA.

SEQ ID NO:16 is the nucleotide sequence of p6, a Plk1-specific siRNA.

SEQ ID NO:17 is the nucleotide sequence of p7, a Plk1-specific siRNA.

SEQ ID NO:18 is the nucleotide sequence of p8, a Plk1-specific siRNA.

SEQ ID NO:19 is the amino acid sequence of the human CENPB polypeptide having Genbank® Reference No. NP_{—}001801.

SEQ ID NO:20 is the amino acid sequence surrounding the serine at position 307 (Ser307) in Plk1.

SEQ ID NO:21 is the amino acid sequence of a fragment of CENPB that can be used as Plk1 substrate.

SEQ ID NO:22 is the amino acid sequence of a fragment of CENPB that can be used as Plk1 substrate.

SEQ ID NO:23 is the amino acid sequence of a fragment of CENPB that can be used as Plk1 substrate.

SEQ ID NO:24 is the amino acid sequence of a fragment of CENPB that can be used as Plk1 substrate.

SEQ ID NO:25 is the amino acid sequence of a fragment of CENPB that can be used as Plk1 substrate.

### DETAILED DESCRIPTION

The assays and polypeptides described herein can be used to identify compounds that modulate Plk1 kinase activity. Plk1 has been shown to induce tumorigenesis in animal models of human cancer and is expressed at high levels in many cancers. Accordingly, compounds that inhibit Plk1 kinase activity are expected to decrease cellular proliferation and ultimately interfere with tumorigenesis.

### Plk1 Substrates

A Plk1 substrate, as used herein, refers to any protein or peptide that is capable of being phosphorylated by Plk1. Methods for determining whether a protein or peptide can be phosphorylated by Plk1, and thus is a Plk1 substrate, are described herein. Plk1 substrates include the proteins APC1, APC3, APC8, B23/Nucleophosmin, BRCA2, Cdc25 (e.g., Cdc25C), Cep55, CHO1/Mklp1, Cohesin, Cyclin B1, Emi1, GRASP65, HSF1, Kizuna, Mklp2/Rabkinesin6, Myt1, Ndd1p (yeast), Nlp, NudC, p53, centromere protein B (CENPB), PICH (Plk1-interacting checkpoint helicase), Pin1, Stathmin/Op18, TCTP, Vimentin, p73 (Koida et al. (2008) J. Biol. Chem. 283:8555-8563), Bora (Seki et al. (2008) J. Cell Biol. 181:65-78), DNA topoisomerase II alpha (Li et al. (2008) J. Biol. Chem. 283:6209-6221), Hbo1 (Wu et al. (2008) Proc. Natl. Acad. Sci. U.S.A. 105:1919-1924), Aurora B, MCAK (Rosasco-Nitcher et al. (2008) Science 319:469-472), Rock2 (Lowery et al. (2007) EMBO J. 26:2262-2273), PBIP1 (Lee et al. (2008) Cell Div. 3:4), BubR1 (Wong ad Fang (2007) J. Cell Biol. 179:611-617), CPEB (Setoyama et al. (2007) Proc. Natl. Acad. Sci. U.S.A. 104:18001-18006), human phosphatase HsCdc14A (Yuan et al. (2007) J. Biol. Chem. 282:27414-27423), small GTP/GDP-binding protein Ran (Feng et al. (2006) Biochem. Biophys. Res. Commun. 349:144-152), and Wee1 (as well as fragments and variants of these proteins that are capable of being phosphorylated by Plk1). In some instances, the Plk1 substrate can be a Plk1 protein (e.g., autophosphorylation).

As detailed in the accompanying Examples, Myt1 polypeptides and fragments thereof can be used as phosphorylation substrates for Plk1. Myt1 amino acid residues that can be subject to phosphorylation by Plk1 include, e.g., serine 426, serine 435, serine 469, and threonine 495. In some instances, a Myt1 polypeptide and fragment thereof can contain a threonine residue corresponding to threonine 495 of Myt1, which is subject to phosphorylation by a Plk1 protein.

Human Myt1 GenBanke Reference NP-004194) is 499 amino acids in length and has the following amino acid sequence:

(SEQ ID NO: 2)

MLERPPALAMPMPTEGTPPPLSGTPIPVPAYFRHAEPGFSLKRP
RGLSRSLPPPPPAKGSIPISRLFPPRTPGWHQLQPRRVSFRGEASETLQS
PGYDPSRPESFFQQSFQRLSRLGHGSYGEVFKVRSKEDGRLYAVKRSMSP
FRGPKDRARKLAEVGSHEKVGQHPCCVRLEQAWEEGGILYLQTELCGPSL
QQHCEAWGASLPEAQVWGYLRDTLLALAHLHSQGLVHLDVKPANIFLGPR
GRCKLGDFGLLVELGTAGAGEVQEGDPRYMAPELLQGSYGTAADVFSLGL
TILEVACNMELPHGGEGWQQLRQGYLPPEFTAGLSSELRSVLVMMLEPDP
KLRATAEALLALPVLRQPRAWGVLWCMAAEALSRGWALWQALLALLCWLW
HGLAHPASWLQPLGPPATPPGSPPCSLLLDSSLSSNWDDDSLGPSLSPEA
VLARTVGSTSTPRSRCTPRDALDLSDINSEPPRGSFPSFEPRNLLSLFED
TLDPT.

Exemplary fragments of Myt1 that can be used as Plk1 substrates include, e.g., amino acids 235-499 of SEQ ID NO:2; amino acids 239-499 of SEQ ID NO:2; amino acids 358-499 of SEQ ID NO: 2;

| | |
|---|---|
| PRNLLSLFEDTLDPT; | (SEQ ID NO: 4) |
| NLLSMFEDTLD; | (SEQ ID NO: 5) |
| PRNLLSMFEDTLDPT; | (SEQ ID NO: 6) |
| NLLSLFEDTLD; | (SEQ ID NO: 7) |
| FEPRNLLSLFEDTLD; | (SEQ ID NO: 8) |
| SFPSFEPRNLLSLFEDTLD; | (SEQ ID NO: 9) |
| PPRGSFPSFEPRNLLSLFEDTLD; | (SEQ ID NO: 10) |
| SFPSFEPRNLLSLFEDTLDPT; | (SEQ ID NO: 11) |
| and | |
| CNLLSLFEDTLDPT. | (SEQ ID NO: 12) |

Also described are Cdc25C polypeptides and fragments thereof that can serve as phosphorylation substrates for Plk1. The human Cdc25C (GenBanke Reference NP_{—}001781) is 473 amino acids in length and has the following amino acid sequence:

(SEQ ID NO: 3)

MSTELFSSTREEGSSGSGPSFRSNQRKMLNLLLERDTSFTVCPD
VPRTPVGKFLGDSANLSILSGGTPKRCLDLSNLSSGEITATQLTTSADLD
ETGHLDSSGLQEVHLAGMNHDQHLMKCSPAQLLCSTPNGLDRGHRKRDAM
CSSSANKENDNGNLVDSEMKYLGSPITTVPKLDKNPNLGEDQAEEISDEL
MEFSLKDQEAKVSRSGLYRSPSMPENLNRPRLKQVEKFKDNTIPDKVKKK
YFSGQGKLRKGLCLKKTVSLCDITITQMLEEDSNQGHLIGDFSKVCALPT
VSGKHQDLKYVNPETVAALLSGKFQGLIEKFYVIDCRYPYEYLGGHIQGA
LNLYSQEELFNFFLKKPIVPLDTQKRIIIVFHCEFSSERGPRMCRCLREE
DRSLNQYPALYYPELYILKGGYRDFFPEYMELCEPQSYCPMHHQDHKTEL
LRCRSQSKVQEGERQLREQIALLVKDMSP.

As detailed in the accompanying Examples, CENPB polypeptides and fragments thereof can be used as phosphorylation substrates for Plk1. CENPB amino acid residues that can be subject to phosphorylation by Plk1 include, e.g., a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB. In some instances, a CENPB polypeptide or fragment thereof can contain a serine residue corresponding to serine 156 of CENPB, which is subject to phosphorylation by a Plk1 protein.

Human CENPB polypeptide (Genbank® Reference No. NP_{—}001801) is 599 amino acids in length and has the following amino acid sequence:

(SEQ ID NO: 19)

MGPKRRQLTFREKSRIIQEVEENPDLRKGEIARRFNIPPSTLST
ILKNKRAILASERKYGVASTCRKTNKLSPYDKLEGLLIAWFQQIRAAGLP
VKGIILKEKALRIAEELGMDDFTASNGWLDRFRRRHGVVSCSGVARARAR
NAAPRTPAAPASPAAVPSEGSGGSTTGWRAREEQPPSVAEGYASQDVFSA
TETSLWYDFLPDQAAGLCGGDGRPRQATQRLSVLLCANADGSEKLPPLVA
GKSAKPRAGQAGLPCDYTANSKGGVTTQALAKYLKALDTRMAAESRRVLL
LAGRLAAQSLDTSGLRHVQLAFFPPGTVHPLERGVVQQVKGHYRQAMLLK
AMAALEGQDPSGLQLGLTEALHFVAAAWQAVEPSDIAACFREAGFGGGPN
ATITTSLKSEGEEEEEEEEEEEEEEGEGEEEEEEGEEEEEEGGEGEELGE
EEEVEEEGDVDSDEEEEEDEESSSEGLEAEDWAQGVVEAGGSFGAYGAQE
EAQCPTLHFLEGGEDSDSDSEEEDDEEEDDEDEDDDDDEEDGDEVPVPSF
GEAMAYFAMVKRYLTSFPIDDRVQSHILHLEHDLVHVTRKNHARQAGVRG
LGHQS.

Exemplary fragments of CENPB that can be used as Plk1 substrates include, but are not limited to, NIPPSTLSTILK (SEQ ID NO:21), TPAAPASPAAVPSEGSGGSTTGWR (SEQ ID NO:22), LAAQSLDTSGLR (SEQ ID NO:23), EAGFGGGPNATITTSLK (SEQ ID NO:24), and SEGSGGSTTGWRAREE (SEQ ID NO:25).

The polypeptides and peptides described herein can, but need not, be isolated. For example, the polypeptides can be expressed (endogenously or exogenously) in a cell for use in cell based assays (see below). The term “isolated” as applied to any of the polypeptides and peptides described herein refers to a polypeptide, or a fragment thereof, that has been separated or purified from components (e.g., proteins or other naturally-occurring biological or organic molecules) which naturally accompany it. Typically, a polypeptide or peptide is isolated when it constitutes at least 60%, by weight, of the protein in a preparation. In some embodiments, the polypeptide or peptide in the preparation consists of at least 75%, at least 90%, or at least 99%, by weight, of the protein in a preparation. Since a polypeptide or peptide that is chemically synthesized is, by its nature, separated from the components that naturally accompany it, a synthetic polypeptide is “isolated.”

As used herein, a “biologically active” fragment or variant of a full-length, wild-type Plk1 substrate (e.g., SEQ ID NOS:2, 3, or 19) is a polypeptide or peptide that retains the ability to function as a phosphorylation substrate for the kinase Plk1. For example, biologically active fragments or variants of a Plk1 substrate or a fragment thereof include, e.g., the amino acid sequences depicted in SEQ ID NOS: 5, 6, 12, or 14. Numerous assays are described herein that allow for a determination of whether a polypeptide functions as a phosphorylation substrate for Plk1.

In some embodiments, a biologically active fragment or variant of a Plk1 substrate (e.g., a biologically active fragment or variant of SEQ ID NOS:2, 3, or 19) has at least one but not more than five amino acids substituted, deleted, or inserted (e.g., as compared to the amino acid sequence of SEQ ID NOS:2, 3, or 19). In some biologically active fragment or variants of a Plk1 substrate, not more than four, three, two, or one amino acids are substituted, deleted, or inserted. Substitutions or deletions can be made at amino acid residues within the Plk1 substrates (e.g., the Plk1 substrates depicted in SEQ ID NOS:2, 3, or 19) other than serine or threonine residues (e.g., threonine 495 of Myt1 or serine 43, serine 156, threonine 169, serine 307, or threonine 396 of CENPB) that are the target of phosphorylation by Plk1.

In some embodiments, a biologically active fragment or variant is prepared by means of conservative substitution at one or more amino acid residues within any Plk1 substrate (e.g., any one of SEQ ID NOS:2, 3, or 19). A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution.

An exemplary biologically active variant of Myt1, which has the GenBank® Reference ID AAB71843, has the following amino acid sequence:

(SEQ ID NO: 14)

MLERPPALAMPMPTEGTPPPLSGTPIPVPAYFRHAEPGFSLKRP
RGLSRSLPPPPPAKGSIPISRLFPPRTPGWHQLQPRRVSFRGEASETLQS
PGYDPSRPESFFQQSFQRLSRLGHGSYGEVFKVRSKEDGRLYAVKRSMSP
FRGPKDRARKLAEVGSHEKVGQHPCCVRLEQAWEEGGILYLQTELCGPSL
QQHCEAWGASLPEAQVWGYLRDTLLALAHLHSQGLVHLDVKPANIFLGPR
GRCKLGDFGLLVELGTAGAGEVQEGDPRYMAPELLQGSYGTAADVFSLGL
TILEVACNMELPHGGEGWQQLRQGYLPPEFTAGLSSELRSVLVMMLEPDP
KLRATAEALLALPVLRQPRAWGVLWCMAAEALSRGWALWQALLALLCWLW
HGLAHPASWLQPLGPPATPPDSPPCSLLLDSSFSSNWDDDSLGPSLSPEA
VLARTVGSTSTPRSRCTPRDALDLSDINSEPPRGSFPSFEPRNLLSMFED
TLDPT.

In some embodiments, a Plk1 substrate or biologically active fragment or variant thereof can be synthesized such that it is phosphorylated on a serine or threonine residue (i.e., a serine or threonine residue that is present in any one of SEQ ID NOS:2-12, 14, or 19-25) such as the phosphorylated peptide as depicted in SEQ ID NO:1 3. In some embodiments, a polypeptide or biologically active fragment or variant thereof can be modified to substitute a serine or threonine residue (i.e., a serine or threonine residue that is present in any one of SEQ ID NOS:2-12, 14, or 19-25) with an amino acid that mimics phophorylation by Plk1 such as an aspartate amino acid residue or a glutamic acid residue. In some instances, a peptide described herein can be modified to substitute a serine or threonine residue of any one of SEQ ID NOS:2-12, 14, or 19-25, with an alternative amino acid (e.g., alanine) so as to create a peptide that cannot be phosphorylated by Plk1 (the modified peptide can be used, e.g., as a control in certain screening assays described herein).

A polypeptide containing the amino acid sequence of any one of SEQ ID NOS:2, 3, or 19, or a biologically active fragment or variant thereof can vary in length. For example, the peptide can contain the amino acid sequence of any one of SEQ ID NOS:2-12, 14, or 19-25 as well as additional amino acid sequences added to the carboxy and/or amino termini. As detailed in the accompanying Examples, the addition of amino acids (e.g., a glutathione S-transferase (GST) moiety) to the amino terminus of a Myt1 (e.g., SEQ ID NO:2) polypeptide or a Cdc25C (e.g., SEQ ID NO:3) yielded polypeptides that function as Plk1 phosphorylation substrates. In instances in which amino acids are added to the carboxy and/or amino termini, the resulting peptide can optionally be less than 100, less than 75, less than 50, less than 40, less than 30, or less than 25 amino acids in length. In some embodiments, the resulting peptide is 20 or fewer or 15 or fewer amino acids in length. In some embodiments, the number of amino acids added to the polypeptide can exceed the number of amino acids originally present in the polypeptide or biologically active fragment or variant thereof.

Polypeptides can be synthesized chemically using standard peptide synthesis techniques. See, e.g., Stewart, et al., Solid Phase Peptide Synthesis (2d ed., 1984). Polypeptides can also be produced by recombinant DNA techniques. For example, a nucleic acid molecule encoding a peptide can be inserted into a vector (e.g., an expression vector) and the nucleic acid can be introduced into a cell. Site-directed mutagenesis can optionally be used, in which a specific nucleotide (or, if desired a small number of specific nucleotides) is changed in order to change a single amino acid (or, if desired, a small number of amino acid residues) in the encoded peptide. Suitable cells for expression of the peptide include, e.g., mammalian cells (such as human cells or CHO cells), fungal cells, yeast cells, insect cells, and bacterial cells (e.g., E. coli). When expressed in a recombinant cell, the cell is cultured under conditions allowing for expression of the peptide. The peptide can optionally be recovered from a cell suspension.

A fusion protein can be prepared that contains the amino acid sequence of a Plk1 substrate or a biologically active fragment or variant thereof (e.g., any one SEQ ID NOS:2-12, 14, or 19-25) and heterologous amino acid sequences. Heterologous, as used herein when referring to an amino acid sequence, refers to a sequence that originates from a source other than the naturally occurring polypeptide from which the peptide is derived. A fusion protein containing a peptide described herein and a heterologous amino acid sequence thus does not correspond in sequence to all or part of a naturally occurring protein. A heterologous sequence can be, for example a sequence used for purification of the recombinant peptide (e.g., FLAG, polyhistidine, hemagluttanin (HA), glutathione-S-transferase (GST), or maltose-binding protein (MBP)). Heterologous sequences can also be proteins useful as diagnostic or detectable markers, for example, luciferase, green fluorescent protein (GFP), or chloramphenicol acetyl transferase (CAT). In some embodiments, the fusion protein contains a signal sequence from another protein. In certain host cells (e.g., mammalian host cells), expression and/or secretion of the peptide can be increased through use of a heterologous signal sequence. In some embodiments, the fusion protein can contain a hapten (e.g., KLH) useful, e.g., in eliciting an immune response (e.g., for antibody generation; see below). Heterologous sequences can be of varying length and in some cases can be a larger sequences than the full-length polypeptides or biologically active fragment or variants thereof to which the heterologous sequences are attached.

In some embodiments, the peptide can be conjugated to a first member of a binding pair (e.g., streptavidin or biotin). As detailed in the following Examples, the addition of a biotin moiety to the amino terminus of Myt1 polypeptides (e.g., SEQ ID NO:9 or SEQ ID NO:11) yielded conjugates that function as Plk1 phosphorylation substrates. Other binding pairs, of which first or second binding members can be conjugated to the polypeptides include, e.g., amylose, maltose, all or part of maltose-binding protein (MBP), glutathione, or all or part of GST.

### Generation of a Phospho-Specific Antibody

Antibodies or antibody fragments that bind to a phosphorylated epitope (e.g., an epitope containing a phosphorylated threonine at amino acid 495 (T495) of Myt1, a phosphorylated serine at position 43 (S43) of CENPB, a phosphorylated serine 156 (S156) of CENPB, a phosphorylated threonine 169 (T169) of CENPB, a phosphorylated serine 307 (S307) of CENPB, or a phosphorylated threonine 396 (T396) of CENPB) can be generated by immunization, e.g., using an animal, or by in vitro methods such as phage display. A polypeptide that includes, e.g., all or part of phosphorylated T495 of Myt1 (e.g., a full-length Myt1 polypeptide phosphorylated at T495) or all or part of a phosphorylated CENPB (e.g., a full-length CENPB polypeptide phosphorylated on any of the serine or threonine residues described above) can be used to generate an antibody or antibody fragment. In some embodiments, a phosphorylated fragment of the Myt1 polypeptide (e.g., a fragment that contains the phosphorylated threonine 495 residue) or CENPB (e.g., any of the phosphorylated serine or threonine residues of CENPB described above) can be used as an immunogen to generate antibodies that can be screened for reactivity to the corresponding phosphorylated protein (e.g., phosphorylated T495 of Myt1, a phosphorylated S43 of CENPB, a phosphorylated S156 of CENPB, a phosphorylated T169 of CENPB, a phosphorylated S307 of CENPB, or a phosphorylated T396 of CENPB). For example, a sequence containing, or consisting of, CNLLSLFED(pT)LDPT (SEQ ID NO:13) can be used to immunize an animal (as described in Example 2).

A peptide can be used to prepare antibodies by immunizing a suitable subject, (e.g., rabbit, goat, mouse, or other mammal) with the peptide. An appropriate immunogenic preparation can contain, for example, a chemically synthesized peptide or a recombinantly expressed peptide. The preparation can further include an adjuvant, such as Freund's complete or incomplete adjuvant, or similar immunostimulatory agent. Immunization of a suitable subject with an immunogenic peptide preparation induces a polyclonal anti-peptide antibody response.

The term antibody as used herein refers to immunoglobulin molecules and immunologically active portions of immunoglobulin molecules (i.e., molecules that contain an antigen binding site that specifically bind to the peptide). An antibody that specifically binds to a peptide described herein is an antibody that binds the peptide, but does not substantially bind other molecules in a sample. Examples of immunologically active portions of immunoglobulin molecules include F(ab) and F(ab′)₂ fragments.

The anti-peptide antibody can be a monoclonal antibody or a preparation of polyclonal antibodies. The term monoclonal antibody, as used herein, refers to a population of antibody molecules that contain only one species of an antigen binding site capable of immunoreacting with the peptide. A monoclonal antibody composition thus typically displays a single binding affinity for a particular peptide with which it immunoreacts.

Polyclonal anti-peptide antibodies can be prepared as described above by immunizing a suitable subject with a peptide immunogen. The anti-peptide antibody titer in the immunized subject can be monitored over time by standard techniques, such as with an enzyme linked immunosorbent assay (ELISA) using immobilized peptide. The titer of phospho-specific antibodies (e.g., anti-T495 Myt1 antibodies or anti-S156 CENPB antibodies) can also be determined using DELFIA® (see Examples below) or LANCE™ assay. If desired, the antibody molecules directed against the peptide can be isolated from the mammal (e.g., from the blood) and further purified by techniques such as protein A chromatography to obtain the IgG fraction. At an appropriate time after immunization, e.g., when the anti-peptide antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), or the EBV-hybridoma technique (Cole et al. (1985), Monoclonal Antibodies and Cancer Therapy, Alan R. Liss, Inc., pp. 77-96). Any of the many well known protocols used for fusing lymphocytes and immortalized cell lines can be applied for the purpose of generating an anti-peptide monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402).

As an alternative to preparing monoclonal antibody-secreting hybridomas, a monoclonal anti-peptide antibody can be identified and isolated by screening a recombinant combinatorial immunoglobulin library (e.g., an antibody phage display library) with a peptide described herein to isolate immunoglobulin library members that bind the peptide.

An anti-peptide antibody (e.g., a monoclonal antibody) can be used to isolate the peptide by techniques such as affinity chromatography or immunoprecipitation. Moreover, an anti- peptide antibody can be used to detect the peptide in screening assays described herein. An antibody can optionally be coupled to a detectable substance such as an enzyme (e.g., horseradish peroxidase, alkaline phosphatase, beta-galactosidase, or acetylcholinesterase), a first or second member of a binding pair (e.g., streptavidin/biotin or avidin/biotin), a fluorescent material (e.g., umbelliferone, fluorescein, fluorescein isothiocyanate, rhodamine, dichlorotriazinylamine fluorescein, dansyl chloride, allophycocyanin (APC), or phycoerythrin), a luminescent material (e.g., europium, terbium), a bioluminescent material (e.g., luciferase, luciferin, or aequorin), or a radioactive materials (e.g., ¹²⁵I, ¹³¹I, ³⁵S, ³²P, ³³P, or ³H).

### Screening Assays

A variety of methods can be used to identify candidate compounds that modulate (inhibit or stimulate) the phosphorylation of a Plk1 substrate (e.g., Myt1, CENPB, or Cdc25C) by Plk1 and/or modulate the interaction between a Plk1 protein and a Plk1 substrate. As Plk1 is known to contribute to the viability and proliferation of cancer cells, such methods are useful in identifying compounds effective for the treatment of cancer.

### Modulation of Phosphorylation of Plk1 Substrates by Plk1

Described herein are methods for identifying a candidate compound that modulates (inhibits or stimulates) phosphorylation of a Plk1 substrate (e.g., the polypeptide of any one of SEQ ID NOS:2 or 3, or a biologically active fragment or variant thereof) by a Plk1 protein. It is understood that compounds that inhibit the phosphorylation of a Plk1 substrate by Plk1 can inhibit the kinase activity of Plk1 directly (e.g., by binding to the Plk1 substrate or the active site of the Plk1 protein) or indirectly (e.g., by inhibiting the expression of Plk1 mRNA or Plk1 protein).

Examples of cell free assay conditions in which Plk1 kinase activity (i.e., the ability of a Plk1 protein to phosphorylate a Plk1 substrate) can be measured are described in detail in the accompanying Examples. Generally, reactions involve the addition of a Plk1 protein and a Plk1 substrate (e.g., the peptide of any one of SEQ ID NOS:2 or 3, or a biologically active fragment or variant thereof) in the presence of ATP and manganese (e.g., MnCl₂) or magnesium (e.g., MgCl₂) in a suitable buffered aqueous medium (e.g., Tris-buffered saline or HEPES), at physiologic temperature (e.g., 37° C.) or room temperature for a suitable amount of time (e.g., 30 minutes, 60 minutes, 90 minutes, or 120 minutes). Kinase reaction conditions and general reaction optimization methodologies are well known in the art. A kinase reaction can be curtailed by the addition of a chelating agent (e.g., EDTA or EGTA), heat inactivation of the kinase, or addition of a strong ionic detergent, e.g., sodium dodecyl sulfate (SDS).

In embodiments where the Plk1 substrate is Plk1 itself (i.e., the autophosphorylation of Plk1), Plk1 protein can be reacted in the presence of ATP and manganese or magnesium without adding further Plk1 substrate to the reaction (see Example 4 below). That is, Plk1 protein can be reacted under conditions effective to permit phosphorylation of the Plk1 itself, prior to detecting or measuring the autophosphorylation of the Plk1.

The Plk1 protein can be, e.g., purified, recombinant enzyme (e.g., recombinantly expressed in a bacterial cell, a yeast cell, an insect cell, or a mammalian cell). The Plk1 protein can also be isolated from a host naturally expressing Plk1 (e.g., a mammalian cell line such as a human cell line such as HeLa cells). For example, endogenous Plk1 protein can be isolated from a cell line that expresses it by immunoprecipation using antibodies specific for Plk1 (see Examples below).

Human Plk1 (GenBank® Reference AAA36659; encoded by GenBank® Reference L19559) is 603 amino acids in length and has the following amino acid sequence: MSAAVTAGKLARAPADPGKAGVPGVAAPGAPAAAPPAKEIPEVLVDPRSRRRYVRG RFLGKGGFAKCFEISDVDTKEVFAGKIVPKSLLLKPHQREKMSMEISIHRSLAHQHVVGFHGFFED NDFVFVVLELCRRRSLLELHKRRKALTEPEARYYLRQIVLGCQYLHRNRVIHRDLKLGNLFLNEDL EVKIGDFGLATKVEYDGERKKTLCGTPNYIAPEVLSKKGHSFEVDVWSIGCIMYTLLVGKPPFETS CLKETYLRIKKNEYSIPKHINPVAASLIQKMLQTDPTARPTINELLGDEFFTSGYIPARLPITCLTIPPR FSIAPSSLDPSNRKPLTVLNKGLENPLPERPREKEEPVVRETGEVVDCHLSDMLQQLHSVNASKP SERGLVRQEEAEDPACIPIFWVSKWVDYSDKYGLGYQLCDNSVGVLFNDSTRLILYNDGDSLQYI ERDGTESYLTVSSHPNSLMKKITLLKYFRNYMSEHLLKAGGNITPRQGDELARLPYLRTWFRTRS AIILHLSNGSVQINFFQDHTKLILCPLMAAVTYIDEKRDFRTYRLSLLEEYGCCKELASRLRYARTM VDKLLSSRSASNRLKAS (SEQ ID NO:1). Human Plk1 proteins having amino acid sequences that differ from SEQ ID NO:1 are described in GenBank® Reference CAA53536 (encoded by GenBank® Reference X75932), GenBank® Reference NP_{—}005021 (encoded by GenBank® Reference NM_{—}005030), and GenBank® Reference P53350 (encoded by GenBank® Reference AAH14846).

The kinase domain of human Plk1 is contained within amino acid residues 1-343 (e.g., amino acid residues 1-343 of SEQ ID NO:1).

A Plk1 protein used in the methods described herein contains the sequence of a naturally occurring Plk1 polypeptide or a fragment or variant thereof that retains serine/threonine kinase activity. A variant Plk1 polypeptide can contain one or more additions, substitutions, and/or deletions relative to the sequence of a naturally occurring Plk1 polypeptide.

In some embodiments, a variant Plk1 polypeptide (i) contains one or more amino acid substitutions, and (ii) is at least 70%, 80%, 85%, 90%, 95%, 98% or 99% identical to SEQ ID NO:1 (or 70%, 80%, 85%, 90%, 95%, 98% or 99% identical to amino acids 1-343 of SEQ ID NO:1). A variant Plk1 polypeptide differing in sequence from SEQ ID NO:1 can include, e.g., one or more amino acid substitutions (conservative or non-conservative), one or more deletions, and/or one or more insertions. In one exemplary embodiment, a biologically active form of Plk1 contains an amino acid substitution at position 210 (e.g., threonine to aspartic acid) of SEQ ID NO:1.

A Plk1 protein can be from any species (e.g., yeast, nematode, insect, plant, bird, reptile, or mammal (e.g., a mouse, rat, dog, cat, goat, pig, cow, horse, whale, or monkey) that expresses a homolog of human Plk1 protein.

The ability of a candidate compound to modulate phosphorylation of a Plk1 substrate (e.g., the polypeptide of any one of SEQ ID NOS:2 or 3, or a biologically active fragment or variant thereof) by a Plk1 protein can be directly measured by adding to a kinase reaction a source of ATP containing an ATP linked to a detectably-labeled gamma-phosphate moiety. The detectable label can be, for example, a radioisotope label (e.g., ³⁵S, ³³P, or ³²P). The ability of a candidate compound to modulate phosphorylation of a Plk1 substrate by a Plk1 protein can be measured by detecting the amount of labeled gamma-phosphate incorporated into the substrate in the presence or absence of the candidate compound. Determining the amount of the labeled phospho-substrate can be accomplished through the use of instrumentation that detects or quantitates radioisotope decay or appropriate autoradiographic film.

The ability of a candidate compound to modulate phosphorylation of a Plk1 substrate (e.g., the peptide of any one of SEQ ID NOS:2 or 3, or a biologically active fragment or variant thereof) by a Plk1 protein can also be determined by analyzing the rate of the substrate's physical passage through a stationary phase matrix (e.g., HPLC or TLC methodology). Following a kinase reaction described herein, samples can be resuspended in an appropriate solvent (or liquid phase) and actively or passively passaged over a stationary phase matrix (e.g., a silica-based gel or plate), which can retard (i.e., increase the retention time of) a modified substrate on the basis of physical properties (e.g., size, hydrophobicity, or charge). The occurrence or non-occurrence of phosphorylation of the Plk1 substrate by a Plk1 protein can be determined by measuring the retention time between the passage of a phosphorylated peptide compared to a non-phosphorylated peptide over the stationary phase matrix. For more details about HPLC methodology, see, for example, Nageswara-Rao et al. (2003) J. Pharm. Biomed. Anal. 33(3):335-377). Alternatively, following the kinase reaction step of the procedure, the mixture can be resuspended in buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using antibodies specific to, e.g., the Plk1 substrate. The extent of phosphorylation of a Plk1 substrate, in the presence or absence of the candidate compound, can be detected by comparing the relative position of the phosphorylated species of substrate with the non-phosphorylated species of substrate. Alternatively, the filter membrane can be subjected to immunoblotting using an antibody specific for a phosphorylated Plk1 substrate (a phospho-specific antibody). For example, the antibody can be one that specifically recognizes phosphorylated T495 of a Myt1 polypeptide or phosphorylated S43, S156, T169, S307, or T396 of a CENPB polypeptide. The extent of phosphorylation of a Plk1 substrate, in the presence or absence of a compound, can be detected by comparing the relative amount of phosphorylated substrate detected by the phospho-specific antibody. Exemplary immunoblotting assays for detecting/measuring Plk1 kinase activity towards a Plk1 substrate are set forth in the accompanying Examples.

The ability of a candidate compound to modulate phosphorylation of a Plk1 substrate (e.g., a peptide of any one of SEQ ID NOS:2, 3, 19, or a biologically active fragment or variant thereof) by a Plk1 protein can also be determined by an enzyme-linked immunosorbent assay (ELISA). A Plk1 kinase reaction can performed in the presence of a Plk1 substrate as detailed herein, followed by addition of a detection antibody that specifically recognizes a phosphorylated residue in a Plk1 substrate (as described above). The extent of phosphorylation of a Plk1 substrate in the presence of the candidate compound can be determined by comparing the amount of antibody bound to the Plk1 substrate (following the kinase reaction) as compared the amount of antibody bound to a control substrate (e.g., a substrate not exposed to the candidate compound and/or the Plk1 protein).

For the purposes of detection, an immunoassay can be performed with an antibody that bears a detection moiety (e.g., a fluorescent agent such a europium, terbium, green-fluorescent protein, or a fluorescent dye). The Plk1 substrate can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the Plk1 substrate to be purified away from reaction components prior to contact with the detection antibody and also allows for subsequent washing of unbound antibody. An example of an immunoassay detection method that can be used to identify a candidate compound that modulates phosphorylation of a Plk1 substrate by a Plk1 protein is the commercially available DELFIA® system of Perkin Elmer Life Sciences (Emeryville, Calif.) and is detailed in the accompanying Examples.

An immunoassay method can alternatively use two detection moieties in fluorescence resonance energy transfer (FRET), which entails the radiationless transfer of energy from a donor molecule to an acceptor molecule. The donor molecule can be a dye or chromophore that initially absorbs energy and the acceptor can be a chromophore to which the energy is subsequently transferred (called a donor/acceptor pair). This resonance interaction occurs over greater than inter-atomic distances, without conversion to thermal energy and without any molecular collision. Due to its sensitivity to distance, FRET is extremely useful in investigating protein-protein interactions and enzymatic reactions.

In one example of a FRET method, a Plk1 substrate (e.g., a peptide of any one of SEQ ID NOS:2, 3, 19, or a biologically active fragment or variant thereof) is conjugated to an energy acceptor molecule and an anti-phospho-specific antibody (e.g., anti-phospho-T495 Myt1 antibody or an anti-phospho-S43, S156, T169, S307, or T396 CENPB antibody) is conjugated to an energy donor molecule. Alternatively, the Plk1 substrate can be conjugated to the energy donor molecule and the anti-phospho-specific antibody can be conjugated to the energy acceptor molecule. The Plk1 substrate can be bound directly to either the FRET energy acceptor or donor or can be conjugated to a first member of a specific binding pair (e.g., biotin/streptavidin or a primary/secondary antibody) with the FRET energy acceptor or donor being conjugated to a second member of the specific binding pair (e.g., streptavidin/biotin or secondary/primary antibody, respectively). For example, the accompanying Examples describe biotin labeled with APC (acceptor) and a secondary antibody labeled with europium (donor). Phosphorylation of the Plk1 substrate by a Plk1 protein can be determined by measuring the amount of FRET following a kinase reaction performed in the presence or absence of a candidate compound. An example of a FRET method that can be used to identify a candidate compound that modulates phosphorylation of a Plk1 substrate by a Plk1 protein is the commercially available LANCE™ assay of Perkin Elmer Life Sciences (Emeryville, Calif.).

An immunoassay for detecting and/or measuring Plk1 kinase activity towards a Plk1 substrate can also involve the use of a “sandwich”-type assay. In these sandwich assays, instead of immobilizing reagents on a solid-phase matrix by the methods described above, a Plk1 substrate can be immobilized on the solid-phase matrix by, prior to exposing the solid-phase matrix to the Plk1 substrate, conjugating a “capture” reagent-specific antibody (polyclonal or monoclonal antibody) to the solid-phase matrix by any of a variety of methods known in the art. The Plk1 substrate is then bound to the solid-phase matrix by virtue of its binding to the capture antibody conjugated to the solid-phase matrix. The procedure is carried out in essentially the same manner described above for methods in which the Plk1 substrate is bound to the solid substrate by techniques not involving the use of a capture antibody. It is understood that in these sandwich assays, the capture antibody should not bind to the same epitope (or range of epitopes in the case of a polyclonal antibody) as the detection antibody (e.g., a phospho-specific T495 Myt1 antibody or a phospho-specific S43, S156, T169, S307, or T396 CENPB antibody). Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope that is either completely physically separated from or partially overlaps with any of the epitopes to which the capture polyclonal antibody binds; or (b) a polyclonal antibody that binds to epitopes other than or in addition to that to which the capture polyclonal antibody binds. The detection antibody can be directly coupled to a detectable label. The detection antibody can be unlabeled and a detectably-labeled second antibody that binds to the detection antibody can be used. Alternatively, the detection antibody can be linked to a first member of a binding pair (e.g., biotin or streptavidin) and the second member of the binding pair can be detectably-labeled.

Methods of detecting and/or for quantifying a detectable label depend on the nature of the label and are known in the art. Appropriate labels include, without limitation, radionuclides (e.g., ¹²⁵I, ¹³¹I, ¹³⁵S, ³H, ³²P, ³³P, or ¹⁴C), fluorescent reagents (e.g., fluorescein, rhodamine, or phycoerythrin), luminescent reagents (e.g., QDOT® nanoparticles supplied by the Quantum Dot Corporation, Palo Alto, Calif.), compounds that absorb light of a defined wavelength, or enzymes (e.g., alkaline phosphatase, luciferase, or horseradish peroxidase). The products of reactions catalyzed by appropriate enzymes can be, without limitation, fluorescent, luminescent, or radioactive or they may absorb visible or ultraviolet light. Examples of detectors include, without limitation, x-ray film, radioactivity counters, scintillation counters, spectrophotometers, colorimeters, fluorometers, luminometers, and densitometers.

The ability of a candidate compound (e.g., a compound identified as a Plk1 kinase inhibitor by a cell-free assay described herein) to modulate Plk1 kinase activity can also be evaluated in a cell-based assay. Cell-based assays can be performed in addition to, sequentially with, or concomitantly with any of the cell-free assays described herein. For example, the effects of a candidate compound on the biological activity of Plk1 can be measured by monitoring the phosphorylation state of an endogenous Plk1 substrate (e.g., Myt1, Emi1, Cdc25C, CENPB, Wee1, BRCA2, p53, Cyclin B1, Nlp, and GRASP65) in cells. The Plk1 substrate can be endogenous (naturally expressed by the cell) or exogenous (e.g., expressed from a plasmid or other recombinant nucleic acid vector encoding a Plk1 substrate or biologically active variant thereof introduced into the cell). The phosphorylation state of the substrate can be measured in intact cells using antibody-mediated immunofluorescence or immunohistochemical techniques. The phosphorylation state of a Plk1 substrate can alternatively be measured, for example, by solubilizing cells and subjecting the solubilized extracts to SDS-PAGE, followed by western blotting with antibodies specific for phosphorylated residues in the Plk1 substrate (as described above). As Plk1 protein levels and Plk1 activity peaks at mitosis, in some embodiments cells can be cultured in the presence of an anti-mitotic (e.g., nocodazole) prior to, or concurrently with, a candidate compound. Alternatively, an antibody that recognizes a non-phosphorylated Plk1 substrate can also be used to detect changes in protein mobility consistent with protein modification (e.g., phosphorylation) (also see above). For example, the phosphorylation state of endogenous Myt1 in the presence and absence of a candidate compound can be determined by analysis of lysates prepared from cells cultured with and without the compound (see Examples). A reduced amount of phosphorylation of Myt1 by Plk1 in the presence of a candidate compound as compared to in the absence of the candidate compound indicates that the candidate compound is a compound that inhibits Plk1. In another example, the phosphorylation state of endogenous CENPB in the presence and absence of a candidate compound can be determined by analysis of lysates prepared from cells cultured with and without the compound. A reduced amount of phosphorylation of CENPB by Plk1 in the presence of a candidate compound as compared to in the absence of the candidate compound indicates that the candidate compound is a compound that inhibits Plk1.

### Modulation of an Interaction Between Plk1 and CENPB

The disclosure also features methods for identifying a candidate compound that modulates (inhibits or stimulates) an interaction between a Plk1 protein and a Plk1 substrate (e.g., a CENPB protein or a fragment or variant thereof that binds to Plk1). It is understood that compounds that inhibit the interaction of a Plk1 protein and, e.g., a CENPB protein can inhibit the interaction between a Plk1 and CENPB (e.g., by binding to the CENPB protein or the active site of the Plk1 protein) or indirectly (e.g., by (i) inhibiting the expression of Plk1 mRNA or Plk1 protein or (ii) inhibiting the expression of CENPB mRNA or CENPB protein).

Examples of cell-free assay conditions in which inhibition of an interaction between a Plk1 protein and a CENPB protein can be measured are described in detail in the accompanying Examples. Generally, conditions involve contacting, in the presence of a candidate compound, a Plk1 protein and a CENPB protein (e.g., the polypeptide of SEQ ID NO:19, or a biologically active fragment or variant thereof) in a suitable buffered aqueous medium (e.g., Tris-buffered saline or HEPES), at physiologic temperature (e.g., 37° C.) or room temperature for a suitable amount of time (e.g., 30 minutes, 60 minutes, 90 minutes, or 120 minutes).

The CENPB protein, like the Plk1 protein (described above), can be, e.g., purified, recombinant proteins (e.g., recombinantly expressed in a bacterial cell, a yeast cell, an insect cell, or a mammalian cell). The CENPB protein can also be isolated from a host naturally expressing the polypeptides. For example, endogenous CENPB protein can be isolated from a cell line that expresses it by immunoprecipation using antibodies specific for CENPB.

In some embodiments, a Plk1 protein used in the methods contains the sequence of a naturally occurring Plk1 polypeptide or a fragment or variant thereof that retains serine/threonine kinase activity.

In some embodiments, a CENPB protein used in the methods contains the sequence of a naturally occurring CENPB polypeptide. In some embodiments, the CENPB protein can be a biologically active fragment or variant thereof that retains the ability to bind to a Plk1 protein. A biologically active variant of CENPB protein (i) can contain one or more amino acid substitutions and/or (ii) can be at least 70%, 80%, 85%, 90%, 95%, 98% or 99% identical to SEQ ID NO:19. A biologically active variant of CENPB protein differing in sequence from SEQ ID NO:19 can include, e.g., one or more amino acid substitutions (conservative or non-conservative), one or more deletions, and/or one or more insertions. A CENPB protein can be from any species (e.g., yeast, nematode, insect, plant, bird, reptile, or mammal (e.g., a mouse, rat, dog, cat, goat, pig, cow, horse, whale, or monkey) that expresses a homolog of human CENPB protein.

The ability of a candidate compound to modulate an interaction between a Plk1 protein and a CENPB protein (e.g., the polypeptide of SEQ ID NO:19 or a biologically active fragment or variant thereof) can be measured in a variety of cell-based methods (e.g., cell-based in vitro and in situ methods). For example, one method of determining inhibition of the interaction between a Plk1 protein and a CENPB protein uses immunoprecipitation and is described in the accompanying Examples. Briefly, cells expressing both a Plk1 protein and a CENPB protein can be cultured in the presence of an inhibitory compound for a pre-determined period of time (e.g., 5 minutes, 10 minutes, 30 minutes, 1 hour, 2 hours, 4 hour, 6 hour, 8 hours, or 12 hours or more), then washed and harvested from the culture vessel. The cells are then lysed using non-denaturing buffers that preserve protein-protein interactions, for example, buffers containing Nonidet™-40 (NP-40) or Triton® X-100 detergents. The lysates can then be clarified using, for example, centrifugation to remove insoluble debris. Clarified lysates are then subjected to immunoprecipitation by adding to the lysate an antibody specific for either a Plk1 protein or a CENPB protein for a time sufficient to allow for the binding of the antibody to its cognate antigen. Antibody-protein complexes are isolated from the lysate solution by coupling the complexes to solid support matrices. Examples of such solid support matrices include insoluble beads conjugated to anti-IgG antibodies or other antibody-binding reagents, for example, bacterial protein-A or protein-G. Isolated immunocomplexes can then be solubilized in Laemmli buffer (optionally containing a reducing agent such as P-mercaptoethanol or dithiothreitol) and subjected to SDS-polyacrylamide gel electrophoresis (SDS-PAGE). Immunoblotting of the samples using antibodies specific for one or both of a Plk1 or CENPB protein can then be used to determine whether a compound has inhibited the interaction between Plk1 and CENPB. For example, a reduced amount of a CENPB protein in anti-Plk1 antibody immunoprecipitates from cells treated with a compound as compared to the amount of CENPB protein in Plk1 immunoprecipitates from cells not treated with the compound indicates that the compound has inhibited the interaction of the two proteins. Similarly, a reduced amount of a Plk1 protein in anti-CENPB antibody immunoprecipitates from cells treated with a compound as compared to the amount of a Plk1 protein in CENPB immunoprecipitates from cells not treated with the compound indicates that the compound has inhibited the interaction of the two proteins.

Another method of determining inhibition of an interaction between Plk1 and CENPB proteins is an in situ staining method. Immunostaining methods are well known to those of skill in the art and include embodiments where the cells are still viable (e.g., confocal microscopy of live cells) or are fixed cells (e.g., immunohistochemistry). Examples of such methods are set forth in the Examples below. Briefly, antibodies specific for Plk1 and/or CENPB polypeptides are applied (e.g., administered, delivered, contacted) to cells. The antibodies are independently labeled with a different detectable label (e.g., a different colored fluorophore (e.g., rhodamine, texas red, FITC, Green fluorescent protein, Cy3, or Cy5) such that they can be readily and easily distinguished from one another. Use of an appropriate microscope (e.g., a confocal microscope) with the appropriate optical filters can identify the position of the labeled antibodies in a given cell. When each of the positions of the two proteins are determined (i.e., the location of their respective detectable label within the cell as determined by antibody binding), if they are found to occupy the same space, the two proteins are said to co-localize. Thus, when two proteins co-localize in the absence of a compound but do not co-localize in the presence of a compound, this can indicate that the compound has inhibited the interaction between the two proteins. Optionally the cells can be fixed, for example, using paraformaldehyde or formaldehyde, and permeabilized using a detergent (e.g., Triton®-X100) prior to contacting the cells with the antibodies.

It is understood that co-localization of two proteins (e.g., Plk1 and CENPB proteins) can be due to a direct, physical interaction of two proteins or it can be due to the localization of two proteins to a given, defined site in a cell (e.g., the nucleus or centromere of a cell), not necessarily involving a physical association between the two proteins. For example, Plk1 and CENPB proteins can co-localize in the nucleus of a cell, but in the absence of an interaction (e.g., in the presence of an inhibitor of their interaction) between them they can relocalize to distinct regions (e.g., the cytoplasm). In this regard, to define the particular localizations or organelles where localization occurs, it can be useful to use antibodies or other dyes that specifically detect the particular organelles or cellular regions of interest. For example, the DNA of a cell can be stained with a variety of chelating agents such as Hoescht stain or 4′,6-diamidino-2-phenylindole (DAPI).

In addition, as the interaction of Plk1 and CENPB proteins can result in CENPB phosphorylation, modulation of an interaction between a Plk1 protein and a CENPB protein can also be determined by measuring the phosphorylation of CENPB by Plk1. Methods for detecting and/or measuring phosphorylation of CENPB by Plk1 (and inhibition of phosphorylation) are described above.

### Candidate Compounds

Candidate compounds that can be used in the methods described herein include various chemical classes and include small organic molecules having a molecular weight in the range of, e.g., 50 to 2,500 daltons. Candidate compounds can optionally contain functional groups that promote interaction with proteins (e.g., hydrogen bonding) and can include at least an amine, carbonyl, hydroxyl, or carboxyl group (or at least two of the functional chemical groups). Candidate compounds can optionally contain cyclical carbon or heterocyclic structures and/or aromatic or polyaromatic structures (e.g., purine core) substituted with one or more of the above functional groups.

Candidate compounds can also include biomolecules including, but not limited to, peptides, polypeptides, proteins, antibodies, peptidomimetics, saccharides, fatty acids, steroids, purines, pyrimidines, derivatives or structural analogues thereof.

Candidate compounds can also include nucleic acids, for example, nucleic acids that inhibit the mRNA or protein expression of a Plk1 protein, for example, an antisense oligonucleotide that hybridizes to a Plk1 mRNA transcript, or a Plk1-specific small interference RNA (siRNA). Antisense oligonucleotides hybridize to Plk1 transcripts and have the effect in the cell of inhibiting expression of a Plk1. siRNAs homologous to Plk1 coding sequences can be also used to reduce expression of a Plk1 in a cell. See, e.g., Fire et al. (1998) Nature 391:806-811; Romano and Masino (1992) Mol. Microbiol. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017 1026. The disclosures of all these articles are incorporated herein by reference in their entirety. Exemplary Plk1-specific siRNAs (e.g., useful as positive controls in assays described herein) are set forth in the accompanying Examples.

Candidate compounds can be identified from a number of potential sources, including chemical libraries, natural product libraries, and combinatorial libraries comprised of random peptides, oligonucleotides (e.g., small inhibitory RNAs (siRNAs)), or organic molecules. Chemical libraries can consist of random chemical structures, some of which are analogs of known compounds or analogs or compounds that have been identified as hits or leads in other drug discovery screens, while others are derived from natural products, and still others arise from non-directed synthetic organic chemistry. Natural product libraries can include polyketides, non-ribosomal peptides, and variants (non-naturally occurring) thereof. For a review, see Science (1998) 282:63-68. Combinatorial libraries can be composed or large numbers of peptides, oligonucleotides, or organic compounds as a mixture.

Peptide libraries can be prepared by traditional automated synthesis methods or by use of recombinant nucleic acids. Libraries of interest include peptide combinatorial, protein, peptidomimetic, multiparallel synthetic collection, recombinatorial, and polypeptide libraries. For a review of combinatorial chemistry and libraries created therefrom, see Myers (1997) Curr. Opin. Biotechnol. 8:701-707. Identification of candidate compounds through the use of the various libraries permits subsequent modification of the candidate compound hit or lead to optimize the capacity of the hit or lead to modulate phosphorylation of a Plk1 substrate by a Plk1 polypeptide.

Candidate compounds identified herein can be synthesized by any chemical or biological method. The candidate compounds can also be pure, or may be in a heterologous composition, and can be prepared in an assay-, physiologic-, or pharmaceutically-acceptable diluent or carrier. This composition can also contain additional compounds or constituents that do not bind to or modulate the kinase activity of a Plk1 polypeptide.

Small molecule inhibitors of Plk1 kinase activity useful as positive controls in the assays described herein include, e.g., wortmannin, staurosporin, scytonemin, BI2536, and ON01910 (see, e.g., Examples and Stebhardt and Ullrich (2006) Nat. Rev. Cancer 6:321-330).

### Multiplex Format

Any of the screening assays can optionally be performed in formats that allow for rapid preparation, processing, and analysis of multiple reactions. This can be, for example, in multi-well assay plates (e.g., 96 wells or 386 wells). Stock solutions for various agents can be provided manually or robotically, and subsequent pipetting, diluting, mixing, distribution, washing, incubating, sample readout, data collection and/or analysis can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the phosphorylation of a Plk1 substrate in a cell) can utilize ArrayScan® VTI HCS Reader or KineticScan® HCS Reader technology (Cellomics Inc., Pittsburg, Pa.).

### Methods for Designing and Producing a Compound

The present disclosure also features methods for predicting or designing compounds that can physically interact with a Plk1 protein and/or a Plk1 substrate (e.g., CENPB) and potentially thereby inhibit the interaction between these two polypeptides. Such compounds would be useful, e.g., to inhibit the ability of Plk1 to promote cell viability and/or cell proliferation (e.g., through inhibition of Plk1 interaction with and/or phosphorylation of its substrates (e.g., CENPB) and thus in the treatment of cancer. One of skill in the art would know how to use standard molecular modeling or other techniques to identify small molecules that would bind to “appropriate sites” on a Plk1 protein and/or a Plk1 substrate such as CENPB (or, e.g., Myt1 or Cdc25C). One such example is provided in Cheng et al. (2003) EMBO J. 22:5757-5768. Generally, an “appropriate site” on a Plk1 protein or a Plk1 substrate is a site directly involved in the physical interaction between the two molecule types. However, an “appropriate site” can also be an allosteric site, i.e., a region of the molecule not directly involved in a physical interaction with another molecule (and possibly even remote from such a “physical interaction” site) but to which binding of a compound results (e.g., by the induction of a conformational change in the molecule) in inhibition of the binding of the molecule to another molecule.

By “molecular modeling” is meant quantitative and/or qualitative analysis of the structure and function of protein- protein physical interaction based on three-dimensional structural information and protein-protein interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods.

Methods of designing compounds that bind specifically (e.g., with high affinity) to the region of a Plk1 protein that interacts with a Plk1 substrate such as CENPB or the region of a Plk1 substrate that binds to a Plk1 protein typically are also computer-based, and involve the use of a computer having a program capable of generating an atomic model. Computer programs that use X-ray crystallography data are particularly useful for designing such compounds. Programs such as RasMol, for example, can be used to generate a three dimensional model of, e.g., the region of a Plk1 protein that interacts with a Plk1 substrate (e.g., CENPB) or the region of a Plk1 substrate that binds to a Plk1 protein and/or determine the structures involved in, e.g., Plk1-CENPB binding. Computer programs such as Insight (Accelrys, Burlington, Mass.), GRASP (Anthony Nicholls, Columbia University), DOCK (Molecular Design Institute, University of California at San Francisco), and Auto-Dock (Accelrys) allow for further manipulation and the ability to introduce new structures.

Compounds can be designed using, for example, computer hardware or software, or a combination of both. However, designing is often implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object-oriented programming language to communicate with a computer system. However, the programs can be implemented in assembly or machine language, if desired. In any case, the language can be a compiled or interpreted language.

Each computer program can be stored on a storage media or device (e.g., ROM or magnetic diskette) readable by a general or special purpose programmable computer. The computer program can serve to configure and operate the computer to perform the procedures described herein when the program is read by the computer. The methods of designing described herein can also be implemented by means of a computer-readable storage medium, configured with a computer program, where the storage medium so configured causes a computer to operate in a specific and predefined manner to perform the functions described herein.

For example, the computer-requiring steps in a method of designing a compound can involve:

(a) inputting into an input device, e.g., through a keyboard, a diskette, or a tape, data (e.g. atomic coordinates) that define the three-dimensional (3-D) structure of a first molecule (e.g., a Plk1 protein or Plk1 substrate-interacting variant thereof) that is known, or predicted, to bind to a second molecule (e.g., a Plk1 substrate (e.g., a CENPB protein) or Plk1-binding variant thereof) or a molecular complex comprising the first and second molecule; and

(b) determining, using a processor, the 3-D structure (e.g., an atomic model) of : (i) the site on the first molecule involved, or predicted to be involved, in binding to the second molecule; or (ii) one or more sites on the molecular components of molecular complex of interaction between molecular components of the molecular complex.

From the information obtained in this way, one skilled in the art will be able to design and make inhibitory compounds (e.g., peptides, non-peptide small molecules, aptamers (e.g., nucleic acid aptamers) with the appropriate 3-D structure. Compounds can be, e.g., any of those described herein under the section entitled “Candidate Compounds.”

Moreover, if computer-usable 3-D data (e.g., x-ray crystallographic or nuclear magnetic resonance (NMR) data) for a candidate compound are available, the following computer-based steps can be performed in conjunction with computer-based steps:

(a) and (b) described above:

(c) inputting into an input device, e.g., through a keyboard, a diskette, or a tape, data (e.g. atomic coordinates) that define the three-dimensional (3-D) structure of a candidate compound;

(d) determining, using a processor, the 3-D structure (e.g., an atomic model) of the candidate compound; (e) determining, using the processor, whether the candidate compound binds to the site on the first molecule or the one or more sites on the molecular components of the molecular complex; and (f) identifying the candidate compound as compound that inhibits the interaction between the first and second molecule or the between the molecular components of the molecular complex.

The method can involve the additional step of outputting to an output device a model of the 3-D structure of the compound. In addition, the 3-D data of candidate compounds can be compared to a computer database of, for example, 3-D structures (e.g., 3-D structures of a Plk1 protein and/or a Plk1 substrate) stored in a data storage system.

Compounds useful for the invention also may be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917). Compounds and polypeptides of the invention also can be identified by, for example, identifying candidate compounds by computer modeling as fitting spatially and preferentially (i.e., with high affinity) into the appropriate acceptor sites on a Plk1 protein or a Plk1 substrate such as CENPB.

Candidate compounds identified as described above can then be tested in standard cellular or cell-free binding or binding inhibition assays familiar to those skilled in the art. Exemplary assays are described herein.

The 3-D structure of biological macromolecules (e.g., proteins, nucleic acids, carbohydrates, and lipids) can be determined from data obtained by a variety of methodologies. These methodologies, which have been applied most effectively to the assessment of the 3-D structure of proteins, include: (a) x-ray crystallography; (b) nuclear magnetic resonance (NMR) spectroscopy; (c) analysis of physical distance constraints formed between defined sites on a macromolecule, e.g., intramolecular chemical crosslinks between residues on a protein (e.g., International Patent Application No. PCT/US00/1 4667, the disclosure of which is incorporated herein by reference in its entirety), and (d) molecular modeling methods based on a knowledge of the primary structure of a protein of interest, e.g., homology modeling techniques, threading algorithms, or ab initio structure modeling using computer programs such as MONSSTER (Modeling Of New Structures from Secondary and Trtiary Restraints) (see, e.g., International Application No. PCT/US99/11913, the disclosure of which is incorporated herein by reference in its entirety). Other molecular modeling techniques may also be employed in accordance with this invention [e.g., Cohen et al. (1990) J. Med. Chem. 33: 883-894; Navia et al (1992) Curr. Opin. Struct. Biol. 2: 202-210, the disclosures of which are incorporated herein by reference in their entirety]. All these methods produce data that are amenable to computer analysis. Other spectroscopic methods that can also be useful in the methods described herein, but that do not currently provide atomic level structural detail about biomolecules, include circular dichroism and fluorescence and ultraviolet/visible light absorbance spectroscopy. For example, one analysis methodology is x-ray crystallography.

### Methods for Evaluating the Efficacy of an Anti-Plk1 Agent

As detailed in the accompanying Examples, Plk1 protein interacts with and phosphorylates CENPB. Thus, the disclosure features methods for evaluating the efficacy of an anti-Plk1 agent utilizing CENPB as a biomarker. In some embodiments, the methods can be performed, e.g., in whole organisms (e.g., in mammals such as humans). For example, in vivo methods can be used to determine the efficacy of an anti-Plk1 therapy in a subject (e.g., a subject with cancer). In some embodiments, the methods can be performed in cell culture and can be used, e.g., as a screening assay to identify compounds having anti-Plk1 activity in cell-based assays. For example, the methods can be used as a secondary assay (following a cell-free biochemical assay) to determine the cellular activity of inhibitors of Plk1 kinase activity or inhibitors of an interaction between Plk1 and CENPB proteins.

In some embodiments, the methods can include the steps of providing a test biological sample from a subject to whom an anti-Plk1 agent was administered; and detecting phosphorylation of a CENPB protein in the test biological sample, wherein a decreased level of phosphorylation of the CENPB protein as compared to the level in a counterpart biological sample from the subject prior to administration of the anti-Plk1 agent indicates that the anti-Plk1 therapy is effective. An increased level (or no change in the level) of phosphorylation of the CENPB protein in the test biological sample as compared to the counterpart biological sample indicates that the anti-Plk1 therapy is not effective.

In some embodiments, the methods can include the steps of providing a test biological sample from a subject to whom an anti-Plk1 agent was administered; and detecting an interaction between a Plk1 protein and a CENPB protein in the test biological sample, wherein a decreased level of interaction between the Plk1 protein and the CENPB protein as compared to the level in a counterpart biological sample from the subject prior to administration of the anti-Plk1 agent indicates that the anti-Plk1 therapy is effective. An increased level (or no change in the level) of interaction between the Pl1 protein and the CENPB protein in the test biological sample as compared to the counterpart biological sample indicates that the anti-Plk1 therapy is not effective.

As used herein, an anti-Plk1 agent is any agent that is capable of inhibiting Plk1 kinase activity, inhibiting Plk1 expression, inhibiting proper localization of Plk1 protein or mRNA in a cell, or inhibiting the interaction between a Plk1 protein and a Plk1 substrate (e.g., Myt1, CENPB, Cdc25C, or any of the other Plk1 substrates described herein).

Suitable biological samples for the methods described herein include any biological fluid, cell, tissue, or fraction thereof, which includes analyte biomolecules of interest such as Plk1 protein or a Plk1 substrate. A biological sample can be, for example, a specimen obtained from a subject (e.g., a mammal such as a human) or can be derived from such a subject. For example, a sample can be a tissue section obtained by biopsy, or cells that are placed in or adapted to tissue culture. A biological sample can also be a biological fluid such as urine, blood, plasma, serum, saliva, semen, sputum, cerebral spinal fluid, tears, or mucus, or such a sample absorbed onto a paper or polymer substrate. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample.

In some embodiments, the method can include obtaining the test biological sample from the subject and/or obtaining the counterpart biological sample from the subject. In some embodiments, the biological samples can be obtained from a subject, e.g., a subject having, suspected of having, or at risk of developing, a cancer. In some embodiments, the biological samples can be obtained from a subject (e.g., a subject with cancer) before and/or after administration of an anti-Plk1 agent.

Any suitable methods for obtaining the biological samples can be employed, although exemplary methods include, e.g., phlebotomy, swab (e.g., buccal swab), or fine needle aspirate biopsy procedure. Non-limiting examples of tissues susceptible to fine needle aspiration include lymph node, lung, thyroid, breast, and liver. Samples can also be collected, e.g., by microdissection (e.g., laser capture microdissection (LCM) or laser microdissection (LMD)), bladder wash, smear (PAP smear), or ductal lavage.

Methods for obtaining and/or storing biological samples that preserve the activity or integrity of molecules (e.g., a Plk1 protein or a Plk1 substrate) in the sample are well known to those skilled in the art. For example, a biological sample can be further contacted with one or more additional agents such as appropriate buffers and/or inhibitors, including nuclease, protease and phosphatase inhibitors, which preserve or minimize changes in the molecules (e.g., nucleic acids or proteins) in the sample. Such inhibitors include, for example, chelators such as ethylenediamne tetraacetic acid (EDTA), ethylene glycol bis(P-aminoethyl ether) N,N,N1 ,NI-tetraacetic acid (EGTA), protease inhibitors such as phenylmethylsulfonyl fluoride (PMSF), aprotinin, leupeptin, antipain and the like, and phosphatase inhibitors such as phosphate, sodium fluoride, vanadate and the like. Appropriate buffers and conditions for isolating molecules are well known to those skilled in the art and can be varied depending, for example, on the type of molecule in the sample to be characterized (see, for example, Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W. B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography.

For use in the methods described herein, a sample can be in a variety of physical states. For example, a sample can be a liquid or solid, can be dissolved or suspended in a liquid, can be in an emulsion or gel, and can be absorbed onto a material.

Suitable methods for determining whether an anti-Plk1 agent inhibits phosphorylation of CENPB or inhibits an interaction between a Plk1 protein and CENPB are described above.

In some embodiments, the methods can further include the step of determining whether Plk1 protein and/or CENPB protein is present (is expressed) in the test biological sample. Methods for determining the presence or expression of a protein are described in the accompanying Examples and include, e.g., immunoblotting using antibodies specific for Plk1 or CENPB proteins.

In some embodiments, the in vivo methods can also include the step of, prior to obtaining a test biological sample from the subject, administering to the subject the anti-Plk1 agent. The anti-Plk1 agent can be any of those described herein including, e.g., scytonemin, ON01910, or BI 2536. Examples of suitable methods for administering an anti-Plk1 agent (e.g., an inhibitor of Plk1 kinase activity, an inhibitor of Plk1 expression, or an inhibitor of an interaction between Plk1 and Plk1 substrate (e.g., Myt1, CENPB, Cdc25C, or any other substrates described herein) are detailed below.

In some embodiments, the in vivo methods can also include the step of, after determining that the anti-Plk1 agent is not effective, administering to the subject a non-anti-Plk1 agent. Where, e.g., the subject is one with cancer, the non-anti-Plk1 agent can be, but is not limited to, a chemotherapeutic agent, an anti-hormonal therapeutic agent, an immunotherapeutic agent, or a radiation therapy. Chemotherapeutics include, e.g., cisplatin, carboplatin, procarbazine, mechlorethamine, cyclophosphamide, camptothecin, adriamycin, ifosfamide, melphalan, chlorambucil, bisulfan, nitrosurea, dactinomycin, daunorubicin, doxorubicin, bleomycin, plicomycin, mitomycin, etoposide, verampil, podophyllotoxin, tamoxifen, taxol, transplatinum, 5-flurouracil, vincristin, vinblastin, methotrexate, and an analog of any of the aforementioned.

### Methods of Inhibiting an Interaction Between Plk1 and CENPB

Provided herein are in vitro and in vivo methods of inhibiting an interaction between a Plk1 protein and a CENPB protein. Inhibition of this interaction can have general applicability in inhibiting the growth or viability of, e.g., a cancer cell. Inhibition of cell growth can be a reversible inhibition of cell growth or an irreversible inhibition of cell growth (e.g., permanent stasis or senescence, or causing the death of the cell). Where the methods are in vivo, such methods can also be useful in the treatment of cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In some embodiments, the methods of inhibiting an interaction between a Plk1 protein and a CENPB protein can, optionally, include a step of identifying a cell as one expressing a Plk1 protein or CENPB protein. That is, in cell-based or in vivo methods, the cell can be one from the subject's cancer, if present. Such identification can include, for example, identifying whether a cell expresses Plk1 (or CENPB) mRNA or protein. Suitable methods of identifying the expression of protein or mRNA are well known to those of skill in the art, and include, for example, sodium dodecyl sulfate-polyacrylamide gel electrophoresis (SDS-PAGE)/western blotting techniques using antibodies specific for Plk1 or CENPB (for detection of protein), or reverse transcription polymerase chain reaction (RT-PCR) or northern blotting techniques for detection of mRNA expression.

Compounds useful in the methods of inhibiting an interaction between Plk1 and CENPB proteins include any of the compounds described herein (e.g., any of the compounds identified, designed, or generated using a method described herein), or any other compounds with the appropriate inhibitory activity.

In vitro methods for inhibiting an interaction between a Plk1 protein and a CENPB protein can be useful, for example, in scientific studies to investigate the role of Plk1 in the regulation of centromere function or any other scientific studies in which inhibiting the interaction between a Plk1 protein and a CENPB protein is beneficial (e.g., cancer studies). Where the method is a cell-based method, it can also be useful as a further screening step, in e.g., a drug screening cascade, following the biochemical (e.g., a cell-free method of identifying a compound that inhibits the binding of a Plk1 and CENPB protein described above) identification of a compound that inhibits the binding of Plk1 and CENPB proteins. Moreover, it can also serve as a “positive control” in assays to identify compounds with the same activity.

The method can include the steps of: contacting (i) a Plk1 protein or a CENPB-binding variant thereof; (ii) a CENPB protein or a Plk1-binding variant thereof; or (iii) a molecular complex comprising (i) and (ii) with a compound that inhibits the interaction between a Plk1 protein and a CENPB protein. The method can also, optionally, include the step of determining whether the inhibition of an interaction between a Plk1 and CENPB protein occurred. The method can be cell-based, and utilize any of the cells described herein (e.g., see above).

Suitable concentrations of the inhibitory compound can be elucidated through routine experimentation and such optimization is well known to one of skill in the art. Where the contacting occurs in a cell, the cell may be co-cultured with one or more additional therapeutic agents such as chemotherapeutic agents.

Methods for detecting inhibition of an interaction between a Plk1 protein and CENPB protein are described above.

### Methods for Inhibiting Phosphorylation of CENPB by Plk1

The disclosure also features in vitro and in vivo methods of inhibiting the phosphorylation of a CENPB protein by a Plk1 protein. Inhibition of phosphorylation of CENPB by Plk1 can have general applicability in inhibiting the growth or viability of, e.g., a cancer cell. Where the methods are in vivo, such methods can also be useful in the treatment of cancers such as any of those described herein.

In some embodiments, the methods of inhibiting the phosphorylation of a CENPB protein by a Plk1 protein can, optionally, include a step of identifying a cell as one expressing a Plk1 protein or CENPB protein. That is, in cell-based or in vivo methods, the cell can be one from the subject's cancer, if present. Such identification can include, for example, identifying whether a cell expresses Plk1 (or CENPB) mRNA or protein (as described above).

Compounds useful in the methods of inhibiting the phosphorylation of a CENPB protein by a Plk1 protein include any of the compounds described herein (e.g., any of the compounds identified, designed, or generated using a method described herein), or any other compounds with the appropriate inhibitory activity. In some embodiments, peptides that contain the amino acid sequence of any one of SEQ ID NOS:21-25 (or a biologically active variant thereof), or that contain S43, S156, T169, S307, or T396 of CENPB, can be used to inhibit phosphorylation of a CENPB protein by a Plk1 protein. In some embodiments, any one of SEQ ID NOS:4-11 can be used to inhibit phosphorylation of a CENPB protein by a Plk1 protein.

In vitro methods for inhibiting the phosphorylation of a CENPB protein by a Plk1 protein can be useful, for example, in scientific studies to investigate the role of Plk1 in the regulation of centromere function or any other scientific studies in which inhibiting phosphorylation of a CENPB protein by a Plk1 protein is beneficial (e.g., cancer studies). Where the method is a cell-based method, it can also be useful as a further screening step, in e.g., a drug screening cascade, following the biochemical (e.g., a cell-free method of identifying a compound that inhibits the phosphorylation of a CENPB protein by a Plk1 protein as described above) identification of a compound that inhibits the binding of Plk1 and CENPB proteins. Moreover, it can also serve as a “positive control” in assays to identify compounds with the same activity.

The method can include the steps of: contacting (i) a Plk1 protein or a CENPB-binding variant thereof; (ii) a CENPB protein or a Plk1-binding variant thereof; or (iii) a molecular complex comprising (i) and (ii) with a compound that inhibits the phosphorylation of a CENPB protein by a Plk1 protein. The method can also, optionally, include the step of determining whether the inhibition of the phosphorylation of the CENPB protein by the Plk1 protein occurred. The method can be cell-based, and utilize any of the cells described herein (e.g., see above).

Suitable concentrations of the inhibitory compound can be elucidated through routine experimentation and such optimization is well known to one of skill in the art (see also “Pharmaceutical Compositions and Methods of Treatment”). Where the contacting occurs in a cell, the cell may be co-cultured with one or more additional therapeutic agents such as chemotherapeutic agents.

Methods for detecting inhibition of the phosphorylation of a CENPB protein by a Plk1 protein are described above.

### Pharmaceutical Compositions and Methods of Treatment

In vivo methods for inhibiting an interaction between a Plk1 protein and CENPB protein can include the steps of: optionally identifying a subject as having, at risk of developing, or suspected to have a cancer; and/or administering to the subject a compound that inhibits an interaction between a Plk1 protein and a CENPB protein. The method can also include the step of (a) determining if the one or more cancer cells of the subject express a Plk1 and/or CENPB protein and/or (b) determining whether inhibition of an interaction between a Plk1 and CENPB protein occurred.

In some embodiments, a compound that inhibits binding of a Plk1 protein to a CENPB protein is administered to a subject. The subject can be any mammal, e.g., a human (e.g., a human patient) or a non-human primate (e.g., chimpanzee, baboon, or monkey), mouse, rat, rabbit, guinea pig, gerbil, hamster, horse, a type of livestock (e.g., cow, pig, sheep, or goat), a dog, cat, or a whale. Any of the compounds described herein can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration.

A compound (e.g., a compound that inhibits an interaction between a Plk1 and a CENPB protein or a compound that inhibits phosphorylation of a CENPB protein by a Plk1 protein) can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Supplementary active compounds can also be incorporated into the compositions.

A pharmaceutical composition is formulated to be compatible with its intended route of administration. Examples of routes of administration include oral, rectal, and parenteral, e.g., intravenous, intramuscular, intradermal, subcutaneous, inhalation, transdermal, or transmucosal. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The compositions can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL [symbol] (BASF, Parsippany, N.J.) or phosphate buffered saline (PBS). In all cases, the composition must be sterile and should be fluid to the extent that easy syringability exists. It should be stable under the conditions of manufacture and storage and must be preserved against contamination by microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of contamination by microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. Prolonged absorption of the injectable compositions can be facilitated by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to cancer cells) can also be used as pharmaceutically acceptable carriers. These can be prepared according to methods known to those skilled in the art, for example, as described in U.S. Pat. No. 4,522,811.

In some instances, oral or parenteral compositions can be formulated in dosage unit form for ease of administration and uniformity of dosage. Dosage unit form, as used herein, refers to physically discrete units suited as unitary dosages for the subject to be treated; each unit containing a predetermined quantity of active compound calculated to produce the desired therapeutic effect in association with the required pharmaceutical carrier. Dosage units can also be accompanied by instructions for use.

The dose administered to a subject, in the context of the present invention should be sufficient to affect a beneficial therapeutic response in the subject over time. The dose will be determined by the efficacy of the particular compound employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disease being treated, the medical or veterinary professional can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present invention can be administered at a rate determined by factors that can include, but are not limited to, the pharmacokinetic profile of the compound, contraindicated drugs, and the side effects of the compound at various concentrations, as applied to the mass and overall health of the subject. Administration can be accomplished via single or divided doses.

Toxicity and therapeutic efficacy of such compounds can be determined by known pharmaceutical procedures in, for example, cell cultures or experimental animals (animal models of cancer, e.g., colon, breast, prostate, or lung cancer models). These procedures can be used, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in to minimize potential damage to normal cells (e.g., non-cancerous cells) and, thereby, reduce side effects.

The data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For a compound used as described herein (e.g., for treating cancer in a subject), the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high-performance liquid chromatography (HPLC).

As defined herein, a therapeutically effective amount of a compound (i.e., an effective dosage) is an amount of the compound that is capable of producing a medically desirable result (e.g., decreased proliferation of cancer cells) in a treated animal and can include milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a compound depend upon the potency of the compound with respect to the inhibition of the interaction between a Plk1 protein and a CENPB protein (and/or its effect on the target cell). When one or more of these compounds is to be administered to an animal (e.g., a human) to treat a cancer, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated. One in the art will also appreciate that certain additional factors may influence the dosage and timing required to effectively treat a subject, including but not limited to the severity of the disease or disorder, previous treatments, and other diseases present. Moreover, treatment of a subject with a therapeutically effective amount of a protein, polypeptide, or antibody can include a single treatment or can include a series of treatments.

A compound or pharmaceutical composition thereof described herein can be administered to a subject as a combination therapy with another treatment, e.g., a treatment for a cancer. For example, the combination therapy can include administering to the subject (e.g., a human patient) one or more additional agents that provide a therapeutic benefit to the subject who has, or is at risk of developing, (or suspected of having) a cancer. Thus, the compound or pharmaceutical composition and the one or more additional agents are administered at the same time. Alternatively, the compound can be administered first in time and the one or more additional agents administered second in time. The one or more additional agents can be administered first in time and the compound administered second in time. The compound can replace or augment a previously or currently administered therapy. For example, upon treating with a compound of the invention, administration of the one or more additional agents can cease or diminish, e.g., be administered at lower levels. Administration of the previous therapy can also be maintained. In some instances, a previous therapy can be maintained until the level of the compound (e.g., the dosage or schedule) reaches a level sufficient to provide a therapeutic effect. The two therapies can be administered in combination.

It will be appreciated that in instances where a previous therapy is particularly toxic (e.g., a treatment for cancer with significant side-effect profiles), administration of the compound can be used to offset and/or lessen the amount of the previous therapy to a level sufficient to give the same or improved therapeutic benefit, but without the toxicity.

In some instances, when the subject is administered a compound or pharmaceutical composition of the invention the first therapy is halted. The subject can be monitored for a first pre-selected result, e.g., an improvement in one or more symptoms of a cancer. In some cases, where the first pre-selected result is observed, treatment with the compound is decreased or halted. The subject can then be monitored for a second pre-selected result after treatment with the compound is halted, e.g., a worsening of a symptom of a cancer. When the second pre-selected result is observed, administration of the compound to the subject can be reinstated or increased, or administration of the first therapy is reinstated, or the subject is administered both a compound and first therapy, or an increased amount of the compound and the first therapeutic regimen.

The compound can also be administered with a treatment for one or more symptoms of a disease (e.g., a cancer). For example, the compound can be co-administered (e.g., at the same time or by any combination regimen described above) with, e.g., a pain medication or a treatment for anemia (e.g., Erythropoietin (EPO)).

### Activation of Plk1

To increase the kinase activity of a Plk1 protein described herein, the Plk1 protein can be activated (also referred to as “pre-activated”) prior to its addition to a kinase reaction. Pre-activation can be achieved by incubating the Plk1 protein in an aqueous buffer containing ATP and a divalent cation such as manganese, calcium, nickel, or zinc. In some embodiments, pre-activation of a Plk1 protein increases kinase activity by at least 1.5 fold (e.g., at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or at least 10 fold or more) relative to the kinase activity of unactivated Plk1.

A Plk1 protein (e.g., at least about 10 μg/ml, at least about 15 μg/ml, at least about 20 μg/ml, at least about 30 μg/ml, at least about 40 μg/ml, at least about 50 μg/ml, at least about 60 μg/ml, at least about 70 μg/ml, at least about 80 μg/ml, at least about 90 μg/ml, at least about 100 μg/ml, at least about 110 μg/ml, at least about 120 μg/ml, at least about 130 μg/ml, at least about 140 μg/ml, at least about 150 μg/ml, at least about 160 μg/ml, at least about 165 μg/ml, at least about 170 μg/ml, at least about 180 μg/ml, at least about 190 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or at least about 1 mg/ml of the Plk1 protein) can be incubated in a buffer containing a divalent cation such as manganese, calcium, nickel, or zinc (e.g., at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 12 mM, at least about 15 mM, at least about 20 mM, or at least about 25 mM of the divalent cation) and ATP (e.g., at least about 10 μM ATP, at least about 20 μM ATP, at least about 30 μM ATP, at least about 40 μM ATP, at least about 50 μM ATP, at least about 60 μM ATP, at least about 70 μM ATP, at least about 80 μM ATP, at least about 90 μM ATP, at least about 100 μM ATP, at least about 110 μM ATP, at least about 120 μM ATP, at least about 150 μM ATP, at least about 175 μM ATP, at least about 200 μM ATP, or at least about 250 μM ATP). The divalent cation can optionally be added as a salt such as a chloride or acetate salt, e.g., manganese chloride (MnCl₂) or manganese acetate (Mn(CH₃COO)₂).

A Plk1 protein can be incubated with a divalent cation (e.g., manganese) and ATP for various periods of time (e.g., at least about 1 hour, at least about 2 hours, at least about 3 hours, at least about 4 hours, at least about 5 hours, at least about 6 hours, at least about 8 hours, at least about 1 0 hours, at least about 1 2 hours, at least about 1 5 hours, at least about 20 hours, at least about 25 hours, or at least about 30 hours). Activation of Plk1 with a divalent cation and ATP can be performed at about 4° C. or at temperatures such as about 1° C., about 2° C., about 3° C., about 5° C., about 6° C., about 7° C., about 8° C., about 10° C., about 15° C., about 20° C., or about 25° C. Activation of a Plk1 protein can also be performed at room temperature or at physiologic temperature, e.g., about 37° C. A Plk1 protein described herein can be activated in the absence of a Plk1 substrate (e.g., any Plk1 substrate described herein, with the exception of Plk1 itself, where it is understood that although Plk1 can be a Plk1 substrate (e.g., by autophosphorylation or by trans-phosphorylation of another Plk1 protein by an activated Plk1 protein), Plk1 will always be present in any of the Plk1 activation methods described herein). Alternatively, a Plk1 protein described herein can be activated in the presence of a Plk1 substrate. A Plk1 protein described herein can optionally be activated with a divalent cation (e.g., manganese) and ATP in the further presence of additional components such as a buffer (e.g., HEPES), 2-glycerol phosphate, a detergent (e.g., CHAPS or MOPS), and/or L-cysteine.

### Arrays and Kits

Also featured herein are protein arrays and kits including the protein arrays that are useful in, e.g., detecting (and/or measuring) phosphorylation of a Plk1 substrate by Plk1 and/or an interaction between a Plk1 protein and a Plk1 substrate.

The protein arrays can include one or more (e.g., two, three, four, five, six, seven, eight, nine, 10, 15, or 20 or more) proteins that are subject to phosphorylation by, or can interact with, a Plk1 protein or biologically active fragment or variant thereof. For example, the protein array can include one or more of APC1, APC3, APC8, B23/Nucleophosmin, BRCA2, Cdc25 (e.g., Cdc25C), Cep55, CHO1/Mklp1, Cohesin, Cyclin B1, Emi1, GRASP65, HSF1, Kizuna, Mklp2/Rabkinesin6, Myt1, Ndd1p (yeast), Nlp, NudC, p53, CENPB, PICH (Plk1-interacting checkpoint helicase), Pin1, Stathmin/Op18, TCTP, Vimentin, or Wee1 as well as biologically active fragments and variants of these proteins that are capable of being phosphorylated by, or of interacting with, a Plk1 protein.

In some embodiments, the protein array comprises CENPB and/or Myt1 polypeptide. In some embodiments, the protein array contains more than one CENPB and/or Myt1 polypeptide. For example, the protein array can include one or more of SEQ ID NOS: 2, or 4-12 and/or one or more SEQ ID NOS:19, or 21-25. In some embodiments, the protein array can contain one or more CENPB polypeptides containing a serine residue that corresponds to position 43 of CENPB, on a serine residue that corresponds to position 156 of CENPB, on a threonine residue that corresponds to position 169 of CENPB, on a serine residue that corresponds to position 307 of CENPB, or on a threonine residue that corresponds to position 396 of CENPB. In some embodiments, the protein array can contain a Myt1 polypeptide containing T495 of Myt1.

A protein of the array can include one or more heterologous sequences as described above.

The protein arrays can be attached to a solid support, e.g., a porous or non-porous material that is insoluble. The substrate can be associated with the support in variety of ways, e.g., covalently or non-covalently bound.

A support can be composed of a natural or synthetic material, an organic or inorganic material. The composition of the solid support on which the proteins are attached (either amino or carboxy-terminal attachment) generally depend on the method of attachment (e.g., covalent attachment). Suitable solid supports include, but are not limited to, plastics, resins, polysaccharides, silica or silica-based materials, functionalized glass, modified silicon, carbon, metals, inorganic glasses, membranes, nylon, natural fibers such as silk, wool and cotton, or polymers. The material comprising the solid support can have reactive groups such as carboxy, amino, or hydroxyl groups, which are used for attachment of the polynucleotides. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, or polymethylpentene (see, e.g., U.S. Pat. No. 5,427,779, the disclosure of which is hereby incorporated by reference in its entirety). Alternatively, the proteins can be attached to the solid support without the use of such functional groups.

Each protein (of a plurality of proteins) on an array can be immobilized at predetermined positions such that each protein can be identified by its position.

In some embodiments of any of the arrays described herein, the array of proteins can have less than 50,000 (e.g., less than 40,000; less than 30,000; less than 20,000; less than 15,000; less than 10,000; less than 5,000; less than 4,000; less than 3,000; less than 2,000; less than 1,500; less than 1,000; less than 750; less than 500, less than 200, less than 1 00, or less than 50) different proteins.

The protein arrays can also be conjugated to solid support particles. Many suitable solid support particles are known in the art and illustratively include, e.g., particles, such as Luminex®-type encoded particles, magnetic particles, and glass particles.

Exemplary particles that can be used can have a variety of sizes and physical properties. Particles can be selected to have a variety of properties useful for particular experimental formats. For example, particles can be selected that remain suspended in a solution of desired viscosity or to readily precipitate in a solution of desired viscosity. Particles can be selected for ease of separation from sample constituents, for example, by including purification tags for separation with a suitable tag-binding material, paramagnetic properties for magnetic separation, and the like.

In some embodiments, encoded particles are used. Each particle includes a unique code (such as a bar code, luminescence code, fluorescence code, a nucleic acid code, and the like). Encoding can be used to provide particles for evaluating different Plk1 substrates simultaneously. The code is embedded (for example, within the interior of the particle) or otherwise attached to the particle in a manner that is stable through hybridization and analysis. The code can be provided by any detectable means, such as by holographic encoding, by a fluorescence property, color, shape, size, weight, light emission, quantum dot emission and the like to identify particle and thus the capture probes immobilized thereto. Encoding can also be the ratio of two or more dyes in one particle that is different than the ratio present in another particle. For example, the particles may be encoded using optical, chemical, physical, or electronic tags. Examples of such coding technologies are optical bar codes fluorescent dyes, or other means. In some embodiments, the particle code is a nucleic acid, e.g., a single stranded nucleic acid.

Different encoded particles can be used to detect or measure the phosphorylation of more than one Plk1 substrate by (or the binding of more than one Plk1 substrate to) a Plk1 protein in parallel, so long as the encoding can be used to identify the protein on a particular particle, and hence the presence or amount of phosphorylation or binding to a substrate. A sample can be contacted with a plurality of such coded particles. When the particles are evaluated, e.g., using a fluorescent scanner, the particle code is read as is the fluorescence associated with the particle from any probe used to evaluate modification of the intact substrate associated with the particles.

One exemplary platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Publication Nos. 2004-0179267, 2004-0132205, 2004-0130786, 2004-0130761, 2004-0126875, 2004-0125424, and 2004-0075907 describe exemplary particles encoded by holographic barcodes.

U.S. Pat. No. 6,916,661 describes polymeric microparticles that are associated with nanoparticles that have dyes that provide a code for the particles. The polymeric microparticles can have a diameter of less than one millimeter, e.g., a size ranging from about 0.1 to about 1,000 micrometers in diameter, e.g., 3-25 μm or about 6-12 μm. The nanoparticles can have, e.g., a diameter from about 1 nanometer (nm) to about 100,000 nm in diameter, e.g., about 10-1,000 nm or 200-500 nm.

Also provided are kits containing any of the protein arrays described herein. The kits can, optionally, contain instructions for detecting and/or measuring phosphorylation of a Plk1 substrate by a Plk1 protein or instructions for detecting/measuring an interaction between Plk1 protein and a Plk1 substrate.

The kits can optionally include, e.g., a control sample containing a known amount of a Plk1 protein. In some embodiments, the control sample can contain a known amount of a control protein known to bind to one or more specific proteins present on the array. The control protein can be detectably labeled.

In some embodiments, the kits can include one or more inhibitors of Plk1 kinase activity. For example, the kit can include one or more of scytonemin, ON01910, or BI 2536.

In some embodiments, the kits can include one or more reagents useful for performing kinase reactions. For example, the kits can include one or more of ATP (e.g., detectably-labeled ATP such as γ³²P-ATP), magnesium, or manganese.

The following are examples of the practice of the invention. They are not to be construed as limiting the scope of the invention in any way.

### EXAMPLES

### Example 1

### Expression of Recombinant Human Plk1

For expression of Plk1 in insect cells, the baculoviral vector pDEST10 (encoding the Plk1 protein of SEQ ID NO:1 with an amino-terminal histidine tag; N-terminal His-tag) was transfected into Sf9 cells using the Invitrogen Gateway baculovirus expression system (Invitrogen, Carlsbad, Calif.). Baculovirus containing the Plk1 coding sequence was amplified using standard methods and used to infect Sf9 insect cells for large-scale protein expression. Plk1 protein was purified by sequential chromatography on NiNTA (nickel-nitroltriacetic acid) then HQ/CM (anion/cation exchange tandem columns). His-Plk1 protein was digested with tobacco etch virus (TEV) protease to remove the N-terminal His-tag, followed by further purification on CM and Phenyl 5PW columns. Electron Spray Ionization (ESI)-mass spectrometry and gel electrophoresis was used to confirm that the Plk1 product was >90% pure and subsequent kinase assays using the purified protein confirmed high enzymatic activity.

For the expression of Plk1 in bacteria, the pDEST10-Plk1 construct was used as a template to duplicate the Plk1 cDNA by polymerase chain reaction (PCR). An internal Ncol site in the Plk1 cDNA was mutated to allow use of the restriction enzyme sites Ncol and Xhol in the cloning vector. PCR primers were used to amplify the Plk1 cDNA with incorporation of a C-terminal 6×-His tag. This PCR product was subcloned into the bacterial vector pET16b at the Ncol/Xhol sites. The Plk1 bacterial expression vector was transformed into BL2(DE3)-RIL E. coli cells (Stratagene). For large scale-amplification, one colony was expanded overnight in PM1 media at 25° C. This culture was then used to seed a 10L B. Braun Biotech Biostat® C fermenter (B. Braun Biotech International, GmbH, Melsungen, Germany). Bacterial cells were expanded to an OD(A600) of 5.0 at 25° C. and induced with 1.0 mM Isopropyl β-D-1-thiogalactopyranoside (IPTG) for 4 hours at 25° C. Bacterial cells were then harvested and spun down at the end of the induction, flash frozen in liquid nitrogen, and stored at −80° C. until purification. Plk1 protein was purified from E. coli by lysis in buffer containing protease inhibitors followed by sequential chromatography on NiNTA, HQ/CM, and size-exclusion columns. The final product,C-terminally-His-tagged full length Plk1, was found to be >95% pure and possess high enzymatic activity.

### Example 2

### Generation and Validation of Anti-Phospho-Myt1 (pT495) Polyclonal Antibodies

Polyclonal antibodies to the region around phosphorylated threonine 495 (phospho-T495) on Myt1 were generated. The peptide CNLLSLFED(pT)LDPT (SEQ ID NO:13) was synthesized and conjugated to the hapten Keyhole Limpet Hemocyanin (KLH). The peptide corresponds to the extreme C-terminus of Myt1 from amino acids 487 to 499, with an N-terminal cysteine residue added to allow for the conjugation to the hapten. Two rabbits were immunized with peptide-KLH to induce an antibody response. Anti-sera were collected after bleeds 1 and 2, and after bleeds 3, 4, and 5. Phospho-specific antibodies to the Myt1 peptide were affinity purified from the obtained anti-sera by sequential chromatography on a column (column “A”) conjugated with peptide containing phospho-T495 (CNLLSLFED(pT)LDPT (SEQ ID NO:13)), followed by a column (column “B”) conjugated with a peptide containing non-phospho-T495 (CNLLSLFED(T)LDPT (SEQ ID NO:12)).

To evaluate phospho-specific antibody titer, a standard DELFIA® assay was conducted wherein two biotinylated Myt1 peptides (MYT_{—}5T and MYT_{—}7T; see below) were bound to a streptavidin ELISA plate and incubated with four anti-P-Myt antibody samples at a dilution of 1:20,000. The antibody samples were as follows: “a-P-Myt-A.12” refers to an antibody from bleeds 1 and 2 that was affinity purified on a first column A; “a-P-Myt-B.12” refers to an antibody from bleeds 1 and 2 that was affinity purified on a second column B; “a-P-Myt-A.345” refers to an antibody from bleeds 3, 4, 5 that was affinity purified on a first column A; and “a-P-Myt-B.345” refers to an antibody from bleeds 3, 4, 5 that was affinity purified on second column B. Specific binding of the antibodies to the substrates was detected using europium-labeled secondary antibody at a dilution of 1:5000. Although each of the antibody preparations tested in the DELFIA® assay against MYT_{—}5T (biotinylated amino acid sequence NLLSLFEDTLD (SEQ ID NO:7)) and MYT_{—}7T (biotinylated amino acid sequence SFPSFEPRNLLSLFEDTLD (SEQ ID NO:9)) produced a higher count level as compared to no peptide, the “a-P-Myt-A.12” appeared to have the highest titer (see Table 1).

TABLE 1

| Antibody Preparation/ | | | |
|---|---|---|---|
| Peptide Tested | noPept | MYT_5T | MYT_7T |
| a-P-Myt-A.12 | 1.7 | 1618 | 2255 |
| a-P-Myt-B.12 | 1.7 | 835 | 172 |
| a-P-Myt-A.345 | 1.0 | 407 | 335 |
| a-P-Myt-B.345 | 0.8 | 340 | 218 |

Values represent signal to noise ratio, calculated from a Plk1 kinase reaction using raw counts in the presence of ATP divided by the raw counts in the absence of ATP. Higher values indicate stronger interaction with phosphorylated product as compared to an unphosphorylated peptide, resulting from direct Plk1 phosphorylation of the peptide.

### Example 3

### Recombinant Plk1 Phosphorylates Myt1 Peptide Using DELFIAO Assay Format

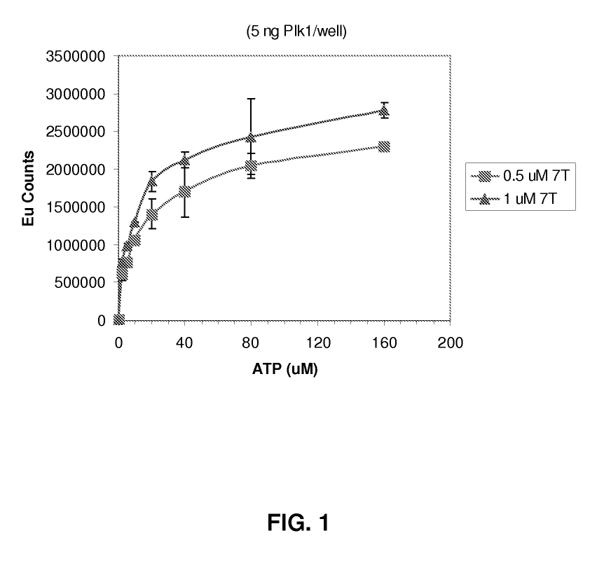

A plate-based DELFIA® assay was developed to detect inhibitors of Plk1 enzyme activity. Recombinant Plk1 enzyme (purified from bacteria; see above) was diluted in PLK1-Buffer D (PKB-D) (20 mM HEPES, 10 mM MgCl₂, 5 mM 2-glycerol phosphate) to a target amount of 5 ng/well (from a working stock of 3.7 nM or 256 ng/ml) and 19.5 μl of this dilution (˜2.9 nM or 200 ng/ml final concentration in the reaction) was added to each well of a 384-well streptavidin-coated assay plate (Perkin Elmer, Waltham, Mass.). Next, Myt1 peptide (MYT_{—}7T; biotinylated amino acid sequence SFPSFEPRNLLSLFEDTLD (SEQ ID NO:9)) was added to the reaction mixture to a final concentration of either 0.5 μM or 1.0 μM along with varying concentrations of ATP (160, 80, 40, 20, 10, 5, 2.5, or 0 μM ATP). Following addition of ATP and peptide, the reactions were incubated for 90 minutes at room temperature. Subsequently, the plates were washed two times with 75 μl of Tris-buffered Saline and Tween® 20 (TBST) using an automated plate washer. Next, 50 μl of a mixture of primary antibody (anti-phospho-threonine antibody (Cell Signaling Technology, Boston Mass.) diluted to 1:4000 in DELFIA® assay buffer) and secondary antibody (anti-rabbit-IgG-Eu (Perkin Elmer) diluted to 1:4000 in DELFIA® assay buffer) was then added to each well. The antibody mixture was incubated on the plate for 60 minutes at room temperature. Following the incubation, the plates were washed twice with TBST. 50 μl of DELFIA® Enhancement Solution was added to each well and further incubated for 30 minutes at room temperature. The fluorescence produced from each well of the plate was detected and quantitated using a Victor5 microplate reader (Perkin Elmer). Recombinant Plk1 phosphorylated the Myt1 peptide in an ATP-dependent manner (FIG. 1). The phosphorylation of Myt1 peptide by Plk1 was dependent on the concentration of the peptide in the reaction.

### Example 4

### Development of a LANCE™ Assay For Measuring Plk1 Kinase Activity

A plate-based LANCE™ assay was developed to detect inhibitors of Plk1 enzyme activity.

### (1) Enhanced Plk1 Kinase Activity in the Presence of Manganese.

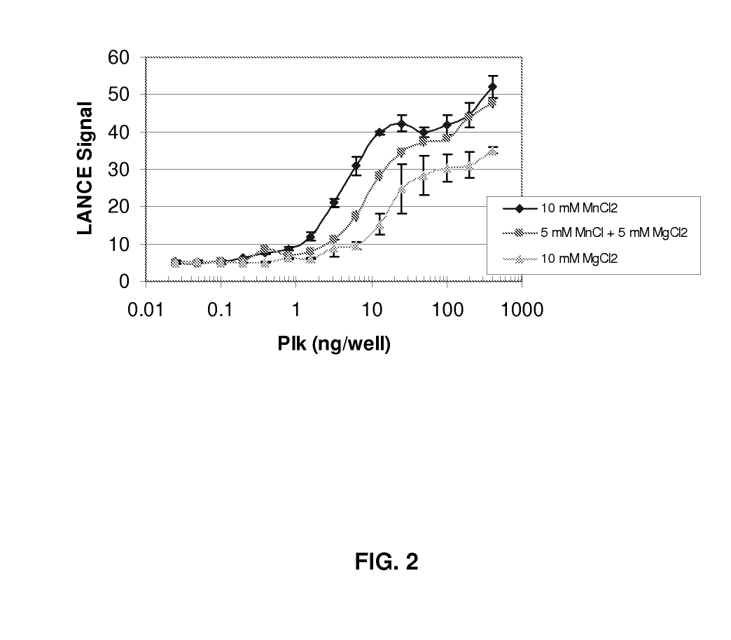

An experiment was performed to evaluate the activity of Plk1 in the presence of manganese (manganese chloride; MnCl₂) and/or magnesium (magnesium chloride; MgCl₂). Recombinant Plk1 enzyme (purified from bacteria; see above) was diluted in PLK1-Buffer L (PKB-L) (20 mM HEPES, 5 mM 2-glycerol phosphate; 0.05% CHAPS detergent and 2 mM L-cysteine) to a target amount of 5 ng/well (from a working stock of 3.7 nM or 256 ng/ml) and 2 μl of this dilution was added to each well of a 384-well streptavidin-coated assay plate (Perkin Elmer). After a 10 minute incubation at room temperature, 6 μl of 1.67× peptide/ATP mixture was added to each well. The reaction was further incubated for 30 minutes at room temperature. The final reaction conditions were: 10 ng (˜14.5 nM or 1 μg/ml) Plk1, 300 nM MYT_{—}13T peptide (MYT_{—}13T; biotinylated amino acid sequence SFPSFEPRNLLSLFEDTLDPT (SEQ ID NO:11)), 10 μM ATP, 20 mM HEPES, 8 mM MgCl2, 0.06 mM MnCl2, 4 mM 2-glycerol phosphate, 1.6 mM L-cysteine, 0.04% CHAPS, 4% DMSO. The reactions were stopped by addition of 5 μl of 60 mM EDTA in LANCE™ detection buffer (final concentration of 10 mM EDTA). Detection of the phosphorylated peptide was accomplished by adding 5 μl of 4× detection mixture (anti-phospho-Myt1 (pT495)) at a final concentration of 1:4800; LANCE-Eu-W1024-labeled-anti-rabbit antibody at a final concentration of 1:2000; and SureLight® streptavidin-APC at a final concentration of 50 nM, all of which were prepared in LANCE™ detection buffer. The reaction mixtures were incubated for 60 minutes at room temperature and then evaluated in a fluorescence microplate reader at 665 and 615 nm dual emission. The signal-to-noise ratio of the reaction was calculated from the ratio of signal produced from reactions with Plk1 protein compared with the signal produced from reactions with no Plk1 protein added. Plk1 kinase activity was enhanced in the presence of manganese as compared to magnesium (FIG. 2).

### (2) Preactivation of Plk1 Enhances Kinase Activity.

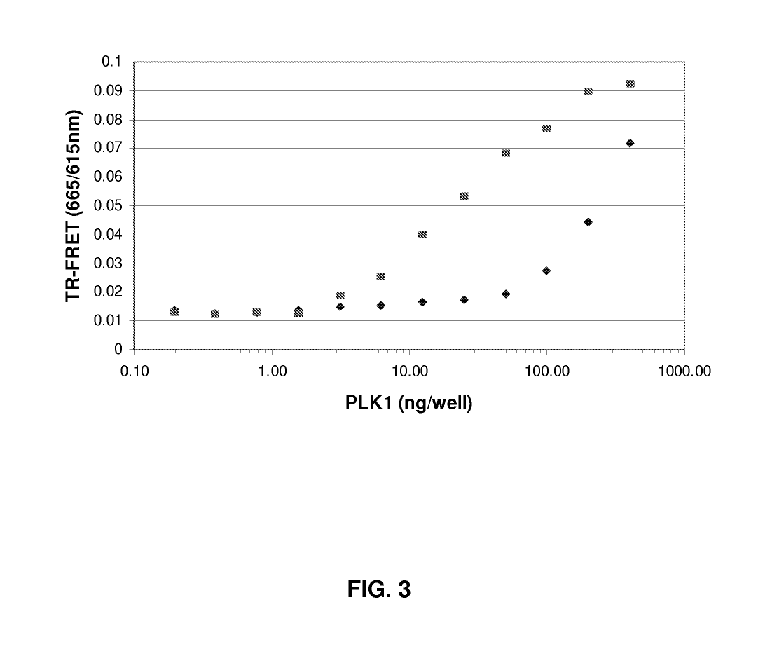

To determine whether pre-incubation of Plk1 with manganese and ATP (pre-activation) increased the activity of Plk1 in the LANCE™ assay, 175 μg of Plk1 protein was either pre-activated for 3 hours at room temperature in 200 μM ATP+10 mM MnCl₂ or left unactivated. Preactivated and unactivated Plk1 were serially diluted from 400 to 0.2 ng per well of a multi-well assay plate and processed in a LANCE™ assay (as described above). Plk1 pre-activated with manganese and ATP was more active in the LANCE™ assay as compared to unactivated Plk1 (FIG. 3).

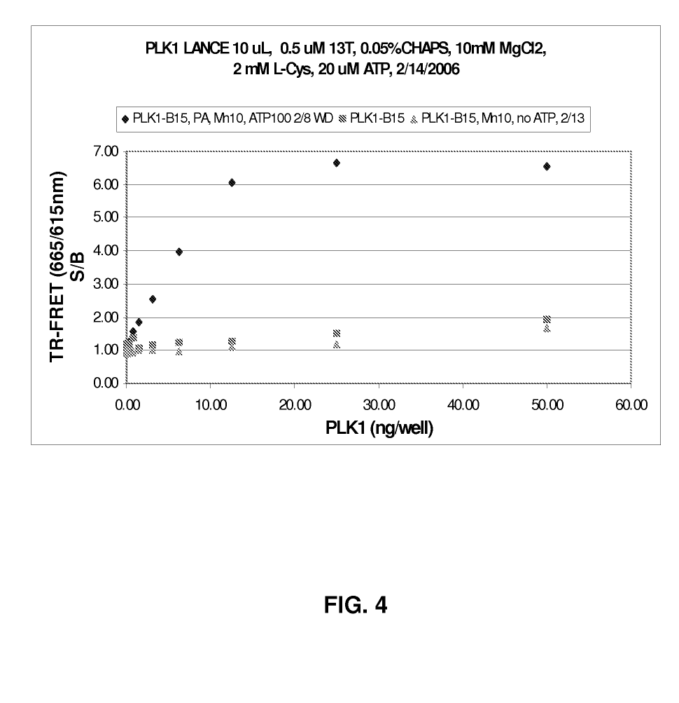

To determine if pre-activation of Plk1 required both manganese and ATP, Plk1 was pretreated with 10 mM MnCl₂ and 100 μM ATP or 10 mM MnCl₂ without ATP. The differently pretreated Plk1 was used in a LANCE™ assay as described above. Plk1 pretreated with manganese and ATP displayed robust activity towards the Myt1 substrate, whereas Plk1 pretreated with only manganese displayed reduced activity comparable to unactivated Plk1 (FIG. 4). These results indicated that preactivation of Plk1 requires both manganese and ATP.

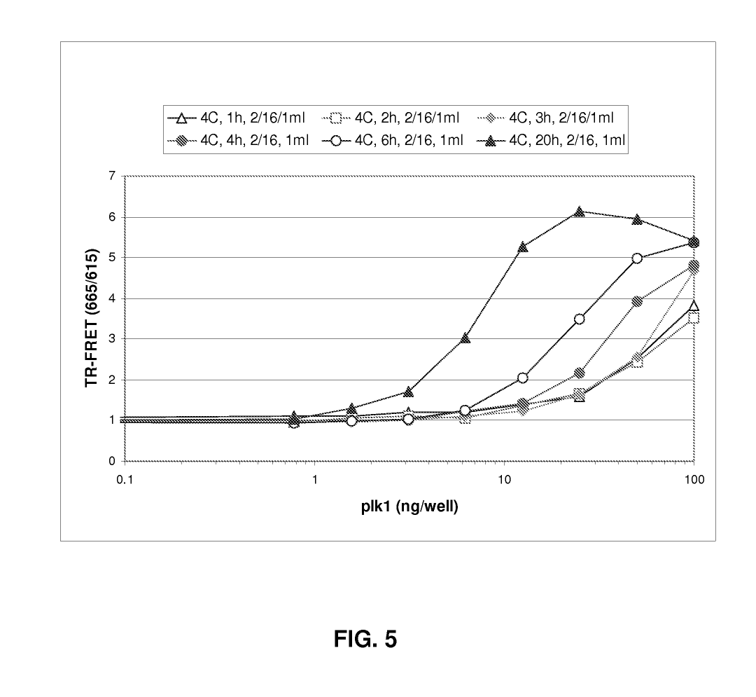

To determine if the amount of time of the preactivation of Plk1 affects its activity in the LANCE™ assay format, Plk1 was preactivated in manganese and 100 μM ATP (as described above) for 1, 2, 3, 4, 6, and 20 hours at 4° C. Preactivated Plk1 incubated for various times was serially diluted from 100 ng per well to 0.75 ng per well and evaluated in the LANCE™ assay. The data indicated that extended preactivation of Plk1 at 4° C. improves its activity in the LANCE™ assay (FIG. 5).

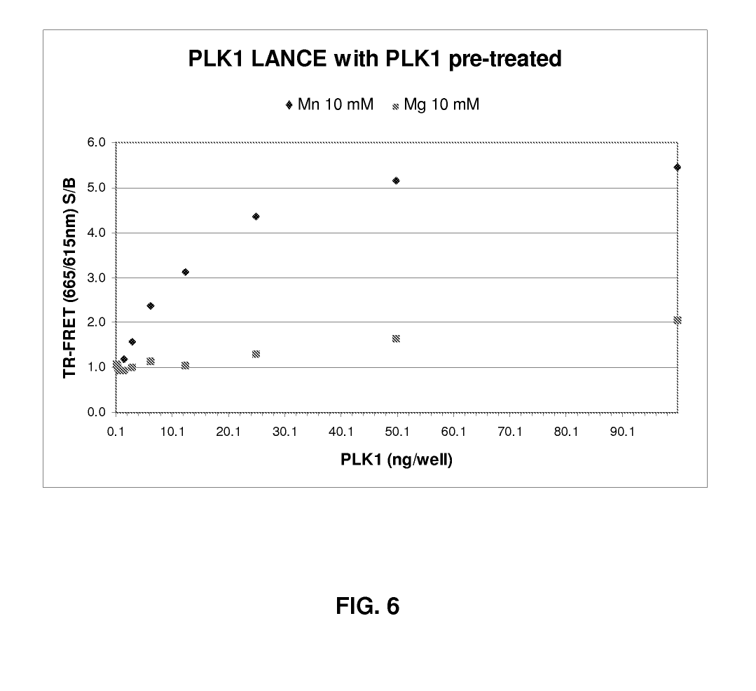

Next, to determine if Plk1 can be activated (pre-activated) using magnesium as well as manganese, various concentrations of Plk1 (0.1 to 100 ng/well) were incubated with 100 μM ATP and either 10 mM MnCl₂ or 10 mM MgCl₂. The differently pretreated Plk1 was used in a LANCE™ assay as above with 5 μM ATP. Plk1 pretreated with manganese and ATP was more active towards the Myt1 substrate as compared to Plk1 pretreated with magnesium and ATP (FIG. 6).

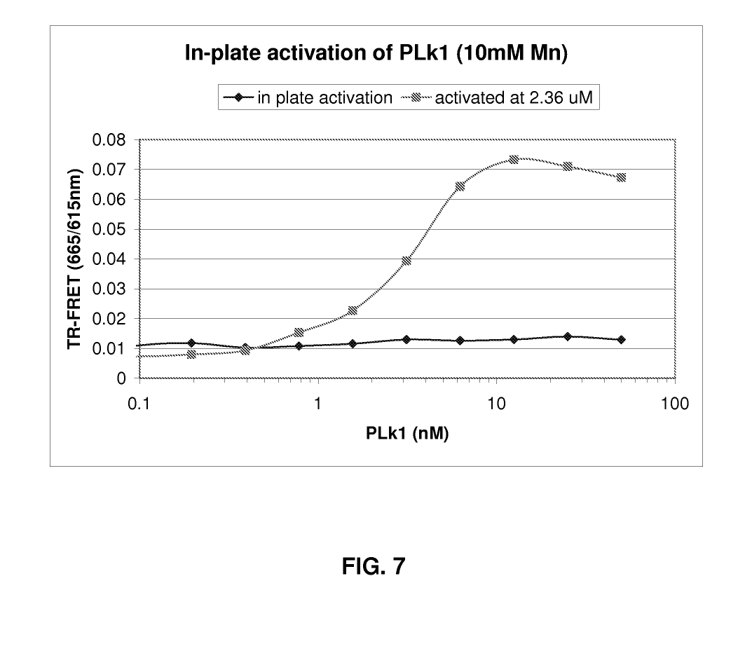

To determine if the concentration of Plk1 affects its pre-activation in the presence of manganese and ATP, Plk1 was pre-activated with 100 ,M ATP and either 10 mM MnCl₂ at a concentration of 165 μg/ml (2.36 μM) or in plate with a range of 0.006˜50 nM. The differentially pre-activated Plk1 was added at various concentrations (50 nM to 0.2 nM) to a LANCE™ assay as described above. Plk1 preactivated at a higher concentration of Plk1 was more active in the LANCE™ assay as compared to Plk1 preactivated at lower concentrations (FIG. 7).

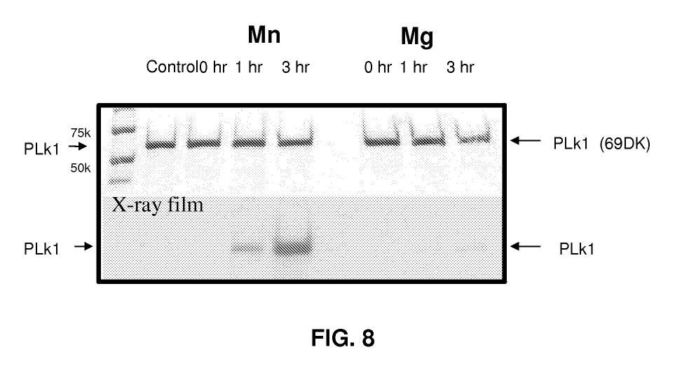

To determine if pre-activation of Plk1 induces the autophosphorylation of Plk1, 165 μg/ml (2.36 μM) of Plk1 was incubated in 20 mM HEPES and 5 mM glycerol phosphate with either 10 mM MnCl2 or 10 mM MgCl2. 10 μM unlabled ATP and 0.033 μM gamma-³³P labeled ATP was also added to the reaction. The reactions were performed at room temperature for 1 or 3 hours. The reactions were stopped by the addition of Laemmli's buffer and subsequently subjected to SDS-PAGE. The SDS-PAGE resolved protein gel was stained with Coomassie blue and dried. The dried gel was then exposed to X-ray film to detect ³³P-labeled (phosphorylated) Plk1 protein. ³³P-labeled Plk1 protein was detected in manganese pre-activated Plk1 reactions at 1 and 3 hours, indicating that pre-activation induces autophosphorylation of Plk1 (FIG. 8).

### (3) The Effect of the Detergent CHAPS on Plk1 Activity.

Various concentrations of unactivated Plk1 (20, 40, 60 ng) were incubated for 60 minutes in buffer containing 10 mM MnCl₂ and 0.05% CHAPS or buffer containing only 10 mM MnCl₂. ATP was also present in the buffer at concentrations at 100 μM or 500 μM. Different concentrations (from 1:500 to 1:2000 dilution) of primary and secondary antibodies were evaluated in the assay. In all cases, the signal-to-noise ratio of Plk1 activity in the LANCE™ assay was found to be higher in the presence of CHAPS detergent (Table 2).

TABLE 2

| | 1:2000 1^{st}/ | | 1:2000 1^{st}/ | | |
|---|---|---|---|---|---|
| | 1:500 2nd | | 1:1000 2nd | | |
| Plk1 | 100 μM | 500 μM | 100 μM | 500 μM | |
| (ng/well) | ATP | ATP | ATP | ATP | Buffer |
| 80 | 4.8 | 4.7 | 5.7 | 5.3 | PKB-L |
| 40 | 3.6 | 3.8 | 4.9 | 4.5 | PKB-L |
| 20 | 1.5 | 1.7 | 2.2 | 2.1 | PKB-L |
| 80 | 9.1 | 9.4 | 10.6 | 11.2 | PKB-L + 0.05% |
| | | | | | CHAPS |
| 40 | 6.8 | 7.1 | 11.2 | 10.7 | PKB-L + 0.05% |
| | | | | | CHAPS |
| 20 | 8.4 | 8.7 | 11.2 | 11.6 | PKB-L + 0.05% |
| | | | | | CHAPS |

### Example 5

### Inhibition of Plk1 Kinase Activity in the DELFIA® and LANCE™ Assays using Small Molecules

To determine the effect of the kinase inhibitors staurosporine, wortmannin, and BI2536 on Plk1 activity using the DELFIA® assay, unactivated Plk1 or manganese/ATP pre-activated Plk1 was evaluated in a DELFIA® assay (as described above) in the presence of various concentrations of the compounds. Pre-activation of Plk1 was conducted with 175 μg of Plk1 in 10 mM MnCl₂ and 100 μM ATP in PKB-D buffer. Plk1 enzyme (unactivated or preactivated) was diluted in PKB-D to a target of 5 ng/well and 19.5 μl was added to each well of a 384-well streptavidin plate. Various concentrations of staurosporine or wortmannin were added to the reaction as a 50× stock in 0.5 μl volume (final concentrations of the compounds ranged from 10 mM to 1 nM). After a 10 minute pre-incubation of compounds with enzyme, 5 μl of 5× biotinylated MYT_{—}13T peptide (SEQ ID NO:11)/ATP mixture was added to each well (5×=5 μM peptide and 50 μM ATP diluted into PKB-D buffer). The final reaction mixtures containing 5 ng (˜2.9 nM or 200 ng/ml) Plk1, 1 uM biotinylated MYT_{—}13T peptide (SEQ ID NO:11), 10 uM ATP, 20 mM HEPES, 10 mM MgCl2, 5 mM 2-glycerol phosphate, 2 mM L-cysteine, 2% DMSO, and various concentrations of inhibitors were incubated for 90 minutes at room temperature. Plates were washed twice with 75 μl of TBST in an automated plate washer. A mixture of primary and secondary antibodies were added and incubated on the plate for 60 minutes at room temperature. Plates were again washed twice with TBST. 50 μl of DELFIA® Enhancement Solution is added (50 μl) and incubated an additional 30 minutes at room temperature. The fluorescence produced from each well of the plate was detected and quantitated using a Victor5 microplate reader (Perkin Elmer). The signal-to-noise ratio (signal over background) was calculated from ratio of paired reactions containing or not containing ATP. The effect of the compounds on Plk1 kinase activity was determined by calculating the decrease in signal from Plk1 reactions with compound as compared to reactions without the compound. All three compounds inhibited Plk1 kinase activity using this assay format. While staurosporine, wortmannin, and BI2536 inhibited Plk1 activity against the Myt1 peptide, the IC₅₀ values for wortmannin and BI2536 were much lower that the IC₅₀ value obtained for staurosporine, indicating that wortmannin and BI2536 were more effective inhibitors of Plk1 (Table 3).

TABLE 3

| | DELFIA ® | DELFIA ® | LANCE ™ | |
|---|---|---|---|---|
| | Unactivated Plk1 | Pre-activated Plk1 | Pre-activated Plk1 | Reported IC₅₀ |
| Wortmannin | 6.1 +/− 3.9 (2) | 17.1 +/− 14 (2) | 15.2 +/− 7.8 (5) | 5.8, 24 |
| Staurosporine | 1891 +/− 1012 (2) | 3537 +/− 3613 (2) | 2040 +/− 847 (5) | 1000 |
| Bl2536 | 13.1 +/− 5.2 (4) | 0.9 +/− 0.1 (4) | 9.4 +/− 4.2 (8) | 0.8 |

IC₅₀ refers to “inhibitory concentration 50%” or the amount of a compound required to reduce the activity of Plk1 to half its maximum.

To determine the effect of the kinase inhibitors staurosporine, wortmannin, and BI2536 on Plk1 activity using the LANCE™ assay, manganese/ATP pre-activated Plk1 was evaluated in a LANCE™ assay in the presence of various concentrations of the compounds. Pre-activation of Plk1 was conducted with 175 μg of Plk1 in 10 mM MnCl₂ and 100 μM ATP in PKB-L buffer. Various concentrations of staurosporine or wortmannin were added to the reaction as a 25× stock freshly diluted in 20% DMSO/20 mM HEPES in a volume of 2 μl (final concentrations of the compounds ranged from 10 mM to 1 nM). MnATP-preactivated-Plk1 enzyme (diluted in PKB-L to a working stock of 5 μg/ml) was added in a volume of 2 μl. After a 10 minute incubation at room temperature, 6 μl of 1.67× peptide/ATP mixture was added to each well. Reactions were then incubated for 30 minutes at room temperature. Final reaction conditions were: 10 ng (˜14.5 nM or 1 μg/ml) Plk1, 300 nM MYT_{—}13T peptide, 10 μM ATP, 20 mM HEPES, 8 mM MgCl2, 0.06 mM MnCl₂, 4 mM 2-glycerol phosphate, 1.6 mM L-cysteine, 0.04% CHAPS, 4% DMSO. Enzyme reactions were stopped by addition of 5 μl of 60 mM EDTA in LANCE™ detection buffer (10 mM EDTA final concentration). To detect the extent of Plk1-dependent phosphorylation of Myt1 peptide, 5 μl of a 4× detection mixture (containing anti-phospho-Myt1 (pT495) at a final concentration of 1:4800; LANCE™-Eu-W1 024-labeled-anti-rabbit secondary antibody at a concentration of 1:2000; and SureLight™ streptavidin-APC at a final concentration of 50 nM, prepared in LANCE™ detection buffer). Plates were incubated for 60 minutes at room temperature. The fluorescence produced from each well of the plate was detected and quantitated using a Victor5 microplate reader (Perkin Elmer) at 665 and 615 nm dual emission. The signal-to-noise ratio (signal over background) was calculated from ratio of paired reactions containing or not containing ATP. The effect of the compounds on Plk1 kinase activity was determined by calculating the decrease in signal from Plk1 reactions with compound as compared to reactions without the compound. While both wortmannin and staurosporine inhibited Plk1 activity against the Myt1 peptide, the IC₅₀ for wortmannin was much lower, indicating that wortmannin was a more effective inhibitor of Plk1 (see Table 3).

The IC₅₀ values obtained for Plk1 under different activation (unactivated and preactivated) and assay format (DELFIA® or LANCE™) conditions were very similar (Table 3). Moreover, pre-activation of Plk1 with manganese and ATP allowed for an ultra-high throughput screening assay format, enabling simultaneous evaluation of large numbers of compounds for inhibitory activity towards Plk1

### Example 6

### An Immunoblot Assay Format for Measuring Plk1 Kinase Activity In Vitro

To determine if Plk1 phosphorylates full-length Myt1 in vitro, Myt1 cDNA obtained from Invitrogen (clone #IOH21301/8ORF01; GenBank® sequence NM_{—}004203) was used as a template to duplicate the Myt1 open reading frame by PCR. Three Myt1 PCR products were created: “Myt1-A” corresponding to full-length Myt1 from amino acids 1-499 (SEQ ID NO:2); “Myt1-B” corresponding to amino acids 239-499 of Myt1; and “Myt1-C” corresponding to amino acids 358-499 of Myt1. Primers with BamHI and Notl restriction enzyme cloning sites at their 5′ ends were used to incorporate these cloning sites into each Myt1 PCR product. The PCR products were subcloned into the bacterial expression vector pGEX-4T-1 at the BamH1/Not1sites, resulting in a recombinant nucleic acid sequence encoding a fusion protein containing an N-terminal GST tagged-Myt1 constructs (e.g., GST-tagged -Myt1-A, Myt1-B, or -Myt1-C). The Myt1 expression vectors were transformed into BL21 *DE3 E. coli cells (Invitrogen) and scaled up to 200 ml cultures. Protein expression was induced by adding 1 mM of IPTG to the cultures. Following induction of expression of the proteins, the three GST-Myt proteins were purified by batch-chromatography on glutathione-sepharose (Amersham Biosciences, Piscataway, N.J.).

The cDNA for Cdc25C was obtained from Invitrogen and subcloned into pDEST15, resulting in an N-terminal GST tag. GST-Cdc25C was expressed in E. coli DH5α (Invitrogen) and purified by chromatography on glutathione-sepharose (as above).

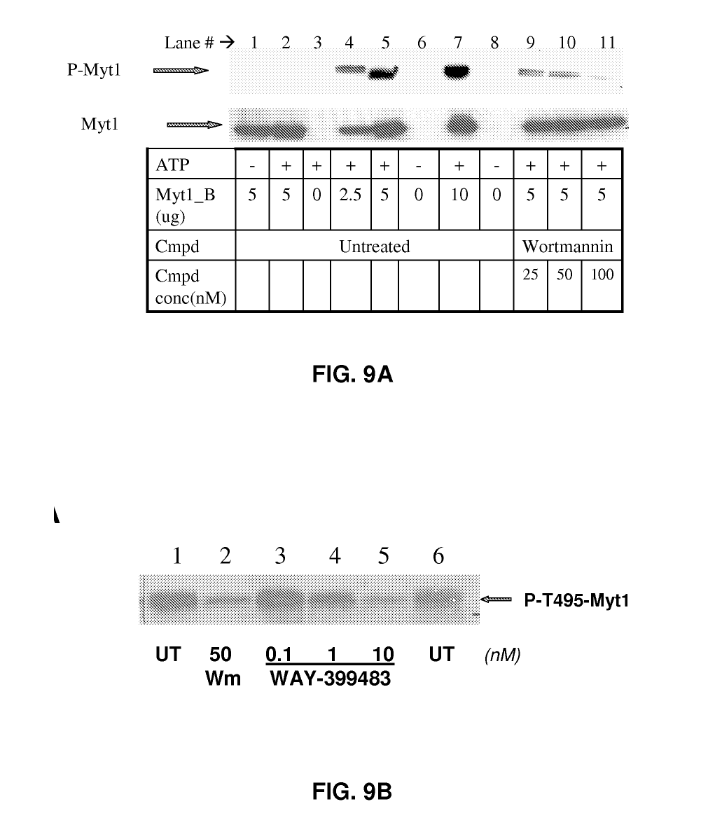

To determine if Plk1 can phosphorylate the GST-Myt1-B truncate, recombinant human Plk1 kinase was incubated with GST-Myt1-B (at 0, 2.5, 5, 10 μg) in the presence or absence of 0, 25, 50, or 100 nM wortmannin or 0.1, 1, or 10 nM BI2536. The reactions were initiated by the addition of 100 μM ATP and allowed to proceed at room temperature for 90 minutes. The reactions were terminated by addition of electrophoresis sample buffer with dithiothreitol (DTT), boiled for 2 minutes, and fractionated by sodium dodecyl sulfate polyacrylamide electrophoresis (SDS-PAGE). SDS-PAGE resolved proteins were immunoblotted with the anti-P-Myt-T495 antibody (described above) to detect phospho-Thr495-Myt1 or anti-Myt1 antibodies to detect total Myt1 protein. The Myt1-B polypeptide was phosphorylated by Plk1 in vitro in an ATP-dependent manner (FIG. 9A). Plk1 phosphorylation of Myt1-B was inhibited by wortmannin and BI2536 (FIGS. 9A and 9B).

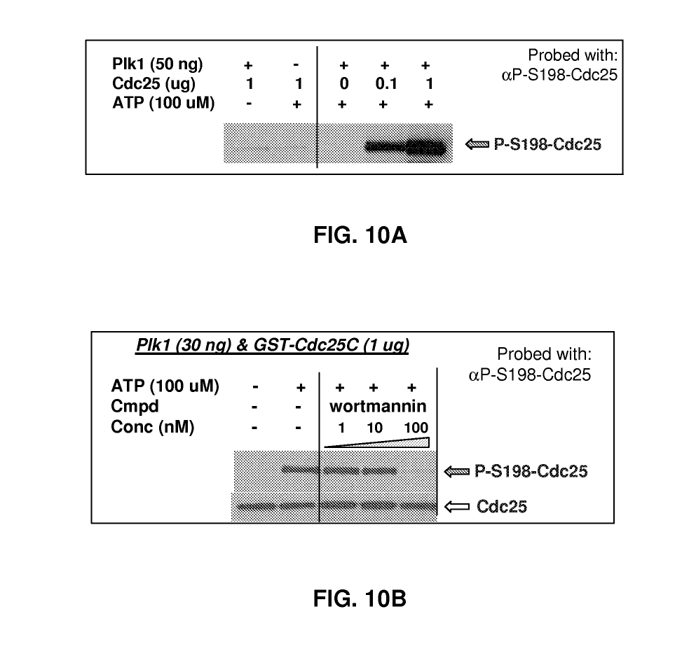

To determine if Plk1 can phosphorylate GST-full-length Cdc25C protein, recombinant human Plk1 kinase was incubated with GST-Cdc25C (at 0, 0.1, and 1 μg). The reactions were initiated by the addition of 100 μM ATP and allowed to proceed at room temperature for 90 minutes. The reaction was terminated by addition of electrophoresis sample buffer with dithiothreitol (DTT), boiled for 2 minutes, and fractionated by sodium dodecyl sulfate polyacrylamide electrophoresis (SDS-PAGE). SDS-PAGE resolved proteins were immunoblotted with an antibody specific for phosphorylated-S198-Cdc25 (Cell Signaling Technology) to detect phosphorylated Cdc25C and then immunoblotted with an antibody specific for Cdc25 protein (to detect total Cdc25 protein). The GST-Cdc25C protein was phosphorylated by Plk1 in vitro in an ATP-dependent manner (FIG. 10A). Plk1 phosphorylation of GST-Cdc25C was also inhibited by wortmannin (FIG. 10B).

### Example 7

### A Plk1 Immune Complex Kinase Assay

In vivo, Plk1 kinase protein levels and activity peak at mitosis. Thus, to obtain active Plk1 from cells, HeLa cells were cultured in 100 mm dishes in the presence or absence of the mitotic phase (M-phase) inhibitor nocodazole for 16 hours. Cells were harvested in LB1 lysis buffer (50 mM HEPES, pH 7.5, 150 mM NaCl, 2 mM EDTA, 1% NP40) and clarified by centrifugation. The protein content of the supernatant was then determined. Plk1 was immunoprecipitated from 1 mg of total protein overnight at 4° C. with gentle rocking by addition of 3-5 μg of a monoclonal anti-Plk1 antibody or a polyclonal anti-Plk1 antibody (obtained from Zymed or Upstate Biolabs, respectively). Protein A/G beads were then added to the immune complexes for 1 hour 4° C. with gentle rocking. The immune complexes were collected by centrifugation and washed twice with LB1 lysis buffer then twice more with PKB-D kinase buffer (see DELFIA® protocol above). To perform in vitro kinase assays using the immunoprecipitated Plk1, PKB-D was then added to each immune complex with 5 μg of GST-Myt1-B and 100 μM ATP, then incubated for 90 minutes at room temperature in the presence or absence of 50 nM wortmannin or 50 nM BI2536. Samples were fractionated by SDS-PAGE and immunoblotted with anti-P-Myt1-T495 as described above for the gel kinase assay. Plk1 immunoprecipitated from HeLa cells cultured in the presence of nocodazole exhibited pronounced kinase activity towards GST-Myt1-B, whereas Plk1 obtained from cells not cultured with nocodazole had less kinase activity towards GST-Myt1-B

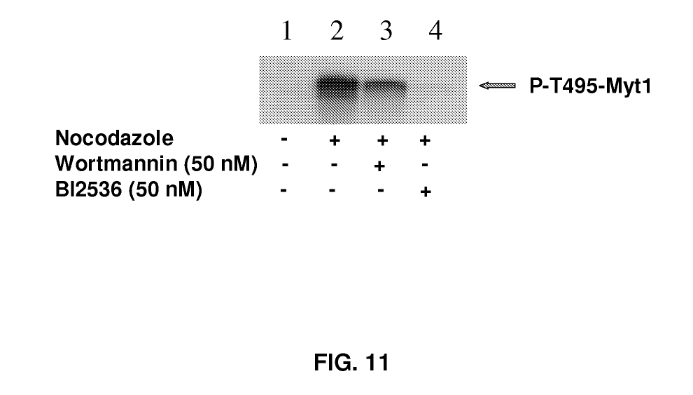

(FIG. 11). In addition, both wortmannin and B12536 inhibited the kinase activity of Plk1 that was immunoprecipitated from cells (FIG. 11).

### Example 8

### Plk1-T210D and Plk-KD Exhibit Increased Specific Activity

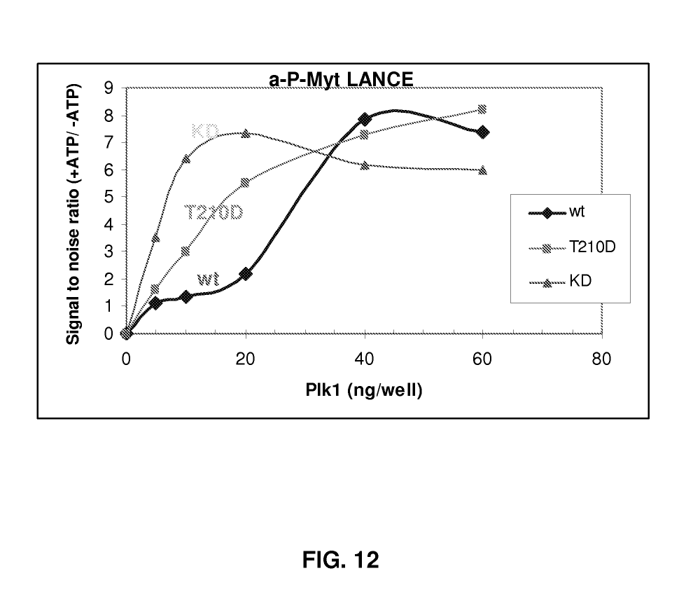

Plk1 is activated in vivo by phosphorylation of threonine 210. To mimic this activation in vitro, the threonine amino acid residue at position 210 of Plk1 was converted to an aspartate residue by site-directed mutagenesis of pET16b-Plk1 (described above) and expressed in E. coli (see above). In addition, a Plk1 protein containing only the kinase domain (Plk-KD; amino acids 1-343) was also generated from the pET16b-Plk1 vector and expressed in bacteria. LANCE™-based kinase assays were performed on these proteins (as described above). These assays demonstrated increased activity/per nanogram enzyme for both proteins compared with full length wild type Plk1 (FIG. 12). These data indicate that LANCE™ assays utilizing the increased kinase activity of Plk1 (kinase domain or T210D mutated) could be used to identify Plk1 inhibitors.

### Example 9

### Plk1 Cellular Assay: Phosphorylation of Plk1 Substrate Proteins

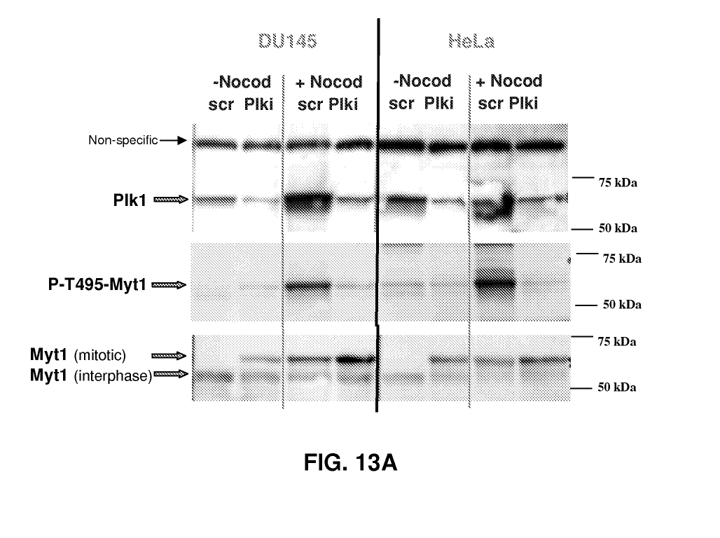

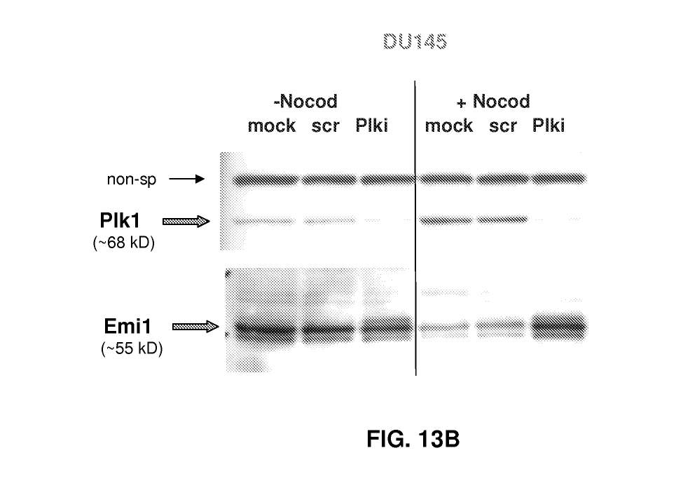

To determine if inhibitors of Plk1 kinase can be identified by observing a change in phosphorylation pattern of one or more known Plk1 substrates in cells, the following experiments were performed. DU145 human prostate cancer cells or HeLa cervical carcinoma cells were treated with or without nocodazole to induce mitotic arrest and increase Plk1 levels. The cells were then incubated with either a scrambled siRNA (scr; negative control) or Plk1-specific siRNA (Plki; pool of siRNA (p5-p8) containing: “p5:” CAACCAAAGTCGAATATGA (SEQ ID NO:15); “p6:” CAAGAAGAATGAATACAGGT (SEQ ID NO:16); “p7:” GAAGATGTCCATGGAAATAT (SEQ ID NO:17); and “p8:” CAACACGCCTCATCCTCT (SEQ ID NO:18)) to reduce Plk1 expression. Cells were then lysed and the proteins were subsequently fractionated using SDS-PAGE. Fractionated proteins were immunoblotted sequentially for Plk1, phospho-T495-Myt1, and Myt1 protein using antibodies specific for each. In the absence of Plki, both DU145 and HeLa cells treated with nocodazole displayed a marked increase in Plk1 protein levels and phosphorylation of Myt1 protein. However, in the presence of Plki, Plk1 protein levels and Myt1 phosphorylation were decreased (see FIG. 13A).

Unlike Myt1, phosphorylation of Emi1 by Plk1 results in the degradation of Emi1. To test whether inhibition of Plk1 stabilizes Emi1 in vivo, DU145 cells were cultured in the presence and absence of nocodazole and further treated with either control-scrambled or Plk1-specific siRNAs (as above). In the absence of Plki, DU145 cells treated with nocodazole contained elevated levels of Plk1 and reduced levels of Emi1 as compared to cells not treated with nocadozole (FIG. 13B). However, cells treated with nocodazole and Plk1 had markedly reduced levels of Plk1 and elevated levels of Emi1 (FIG. 13B). These results indicate that Plk1 inhibitors can be identified in cell-based assays using downstream targets of Plk1 such as Myt1 or Emi1 as indicators.

### Example 10

### Plk1 Phosphorylates CENPB In Vitro

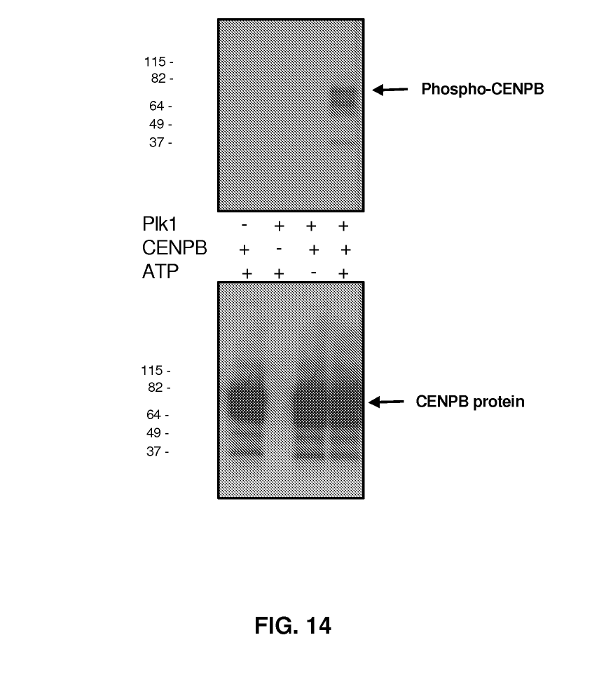

A protein microarray was used to identify centromere protein B (CENPB) as a potential substrate of Plk1. The ability of Plk1 to phosphorylate CENPB was evaluated using a solution-phase kinase assay. Kinase reactions were performed in which 50 ng of recombinant Plk1 (above) was contacted with 1 μg of CENPB (Sf9 insect cell-expressed, BioWorld, Dublin, Ohio) and 100 μM ATP in the a kinase buffer containing 20 mM HEPES/10 mM MgCl₂/5 mM 2-glycerophosphate/0.5 mM L-cysteine. The reactions were allowed to proceed for 90 minutes at 30° C., then resuspended in Lamaelli sample buffer and boiled. The reaction mixtures were subjected to SDS-PAGE and immunoblotting, using an anti-phosphothreonine antibody (Cell Signaling Technologies) and an anti-CENPB (Santa Cruz Biotechnology, Santa Cruz, Calif.). In the presence of ATP, Plk1 phosphorylated CENPB (FIG. 14; top panel, lane 4).

Collectively, these results indicate that Plk1 phosphorylates CENPB.

### Example 11

### Identification of Phosphorylation Sites on CENPB

To determine the specific amino acid residues phosphorylated by Plk1 in vitro, mass spectrometric analysis of Plk1-phosphorylated CENPB was performed. Briefly, a solution-phase kinase assay was performed using recombinant Plk1 (30 nM) and recombinant CENPB (0.5 μg, 303 nM) as described above (Example 10). Reaction mixtures were frozen and subjected to SDS-PAGE followed by in-gel trypsinization. The trypsinized CENPB polypeptides were then evaluated by ion-trap mass spectrometry.

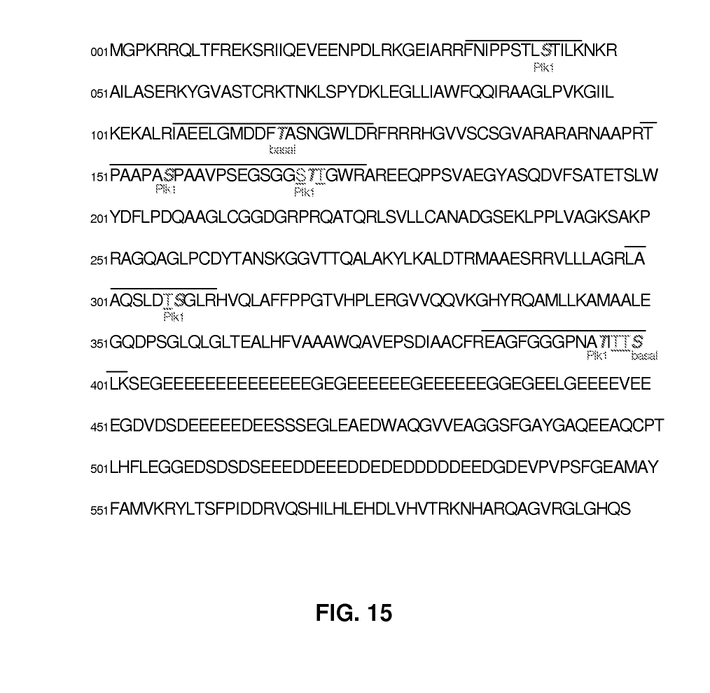

Five residues were determined to exhibit Plk1-dependent phosphorylation: Ser43, Ser156, Thr169, Ser307, and Thr396 (designated “Plk1” in FIG. 15; SEQ ID NO:19). Two sites, Thr117 and Ser400, were determined to be phosphorylated in the absence of Plk1 (designated “basal” in FIG. 15). Notably, the sequence surrounding Ser307 (DTSG; SEQ ID NO:20) corresponds to a reported Plk1 consensus sequence (E/D/Q)-X-(S/T)-(hydrophobic) (Barr et al. (2004) Nature Rev. Mol. Cell Biol. 5:429-440). Amino acid residues proximal to the identified Plk1 target sites (FIG. 15, underlined) may also be phosphorylated but could not be unambiguously identified due to the nature of the analysis. However, these data indicate that at least amino acid residues Ser43, Ser156, Thr169, Ser307, and Thr396 of CENPB can be phosphorylated by Plk1.

### Example 12

### Plk1 and CENPB Co-Localize in Human Cells

To determine if Plk1 and CENPB polypeptides exhibit similar localization (co-localize) in cells, cultured human embryonic kidney (HEK293) cells were fixed and incubated with antibodies specific for either CENPB or Plk1 polypeptides. The binding of the anti-CENPB antibody to CENPB was detected using a secondary antibody labeled with FITC and the binding of the anti-Plk1 antibody to Plk1 was detected using a secondary antibody labeled with rhodamine. The DNA of each cell was stained using DAPI. Both CENPB and Plk1 polypeptides exhibited a punctate staining in the same location as the DNA in mitotic cells. Similar results were also obtained in human cervical carcinoma cells (HeLa cells). These results suggest that Plk1 and CENPB could interact functionally in cells.

### Example 13

### Plk1 Interacts with and Phosphorylates CENPB in Cells

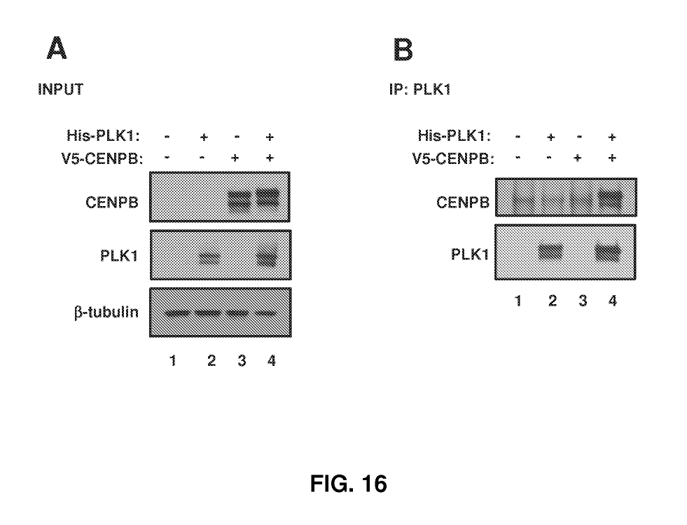

To determine if Plk1 and CENPB physically interact in cells, co-immunoprecipitation experiments were performed in human lung carcinoma cells (H1299 cells) transiently transfected with plasmids encoding His-tagged Plk1 and/or V5-tagged CENPB polypeptides (FIG. 16A). Cells transiently expressing the polypeptides were harvested, lysed, and subjected to immunoprecipitation using anti-Plk1 antibodies. The immunoprecipitates were subjected to SDS-PAGE and immunoblotted using antibodies specific for CENPB (FIG. 16B, top panel) or Plk1 (FIG. 16B, lower panel). V5-epitope tagged CENPB was immunoprecipitated in cells co-expressing both Plk1 and CENPB. To confirm that each of the proteins were expressed in the cells, lysates from each of the transfected cell populations, and a control, non-transfected cell population, were also evaluated by SDS-PAGE/immunoblotting. These data indicate that CENPB interacts with Plk1 in cells.

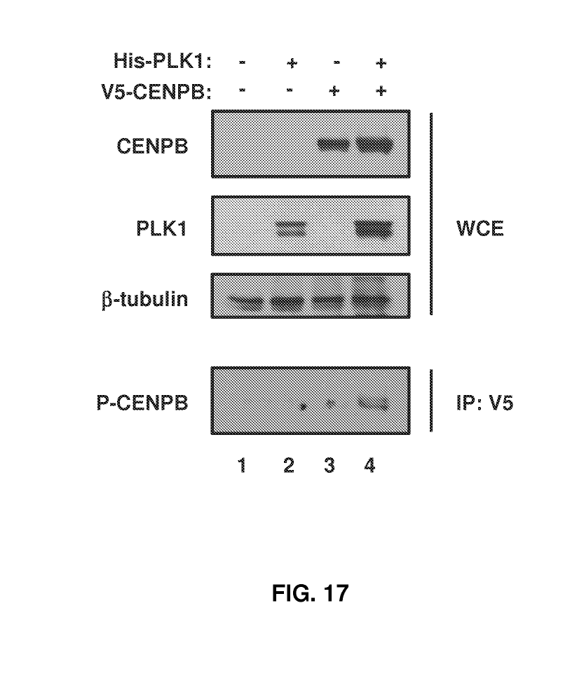

To determine if Plk1 phosphorylates CENPB in cells, His-Plk1 and V5-CENPB were either transiently co-expressed or individually expressed in H1299 cells as described above. Following the transfection and expression, cells were harvested, lysed, subjected to SDS-PAGE, and immunoblotted using antibodies specific for CENPB, Plk1, or β-tubulin as a control. Whole cell extracts were also subjected to immunoprecipitation using anti-V5 antibodies prior to SDS-PAGE and immunoblotting using an anti-phosphothreonine antibody. Phosphorylated CENPB, as determined using the anti-phosphothreonine antibody, was detected in immunoprecipitates from lysates of cells co-expressing CENPB and Plk1 (FIG. 17; lower panel). These results indicate that Plk1 promotes CENPB phosphorylation in cells.

### Example 14

### Plk1 Kinase Activity is Inhibited by BI2536

Inhibition of Plk1 activity by BI2536 was assayed by measuring the amount of phosphorylation of Myt1 at threonine 495, or by measuring the amount of stabilization of Emi1. In both cases, DU145 cells were treated overnight with nocodazole to synchronize in mitotic phase and to induce Plk1 activity. Following treatment with nocodazole, different amounts of BI2536 (0, 1, 10, 100, and 1000 nM) were added.

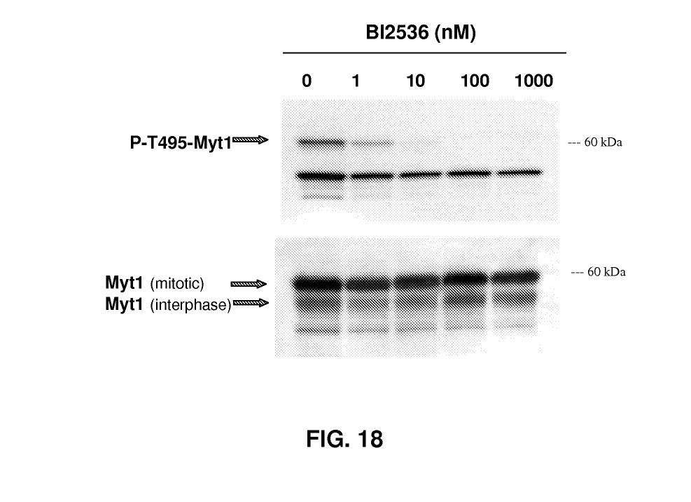

The ability of BI2536 to inhibit phosphorylation of Myt1 protein at site T495 was determined by using anti-phospho-T495-Myt1 antibody on total protein fractionated after treatment with BI2536 inhibitor. Briefly, after incubating the cells for 3 hours in the presence of the inhibitor, the cells were lysed, and total protein was fractionated by SDS-PAGE. Immunoblot analysis was performed using a phospho-specific anti-pT495 Myt1 antibody (see FIG. 18 top panel) and an anti-Myt1 antibody (FIG. 18 bottom panel). These data show a dose-dependent inhibition of phosphorylation of threonine 495 (T495) on Myt1 by Plk1. Treatment with 1 nM BI2536 resulted in over 50% inhibition or phosphorylation of threonine 495 on Myt1 with no change in the total Myt1 protein levels. Therefore, specific phosphorylation of T495 on Myt1 can be monitored to evaluate the efficacy of inhibitors of Plk1 kinase activity in cells. Similar data was observed in multiple cell lines and at various treatment times.

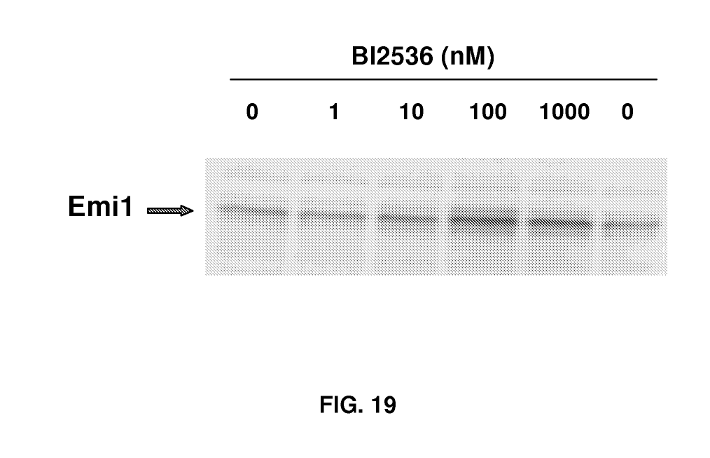

The effect of BI2536 on protein levels of the Plk1 substrate, Emi1, was determined using an anti-Emi1 antibody. Briefly, after incubating the cells for 6 hours in the presence of the inhibitor, the cells were lysed, and total protein was fractionated by SDS-PAGE. Immunoblot analysis was performed using an anti-Emi1 antibody. As depicted in FIG. 19, inhibition of Plk1 with a small molecule kinase inhibitor results in stabilization of Emi1 protein. This is a similar result as observed with Plk1 RNA interference experiments (see FIG. 13B),. These data suggest that Emi1 protein levels can be monitored to evaluate the efficacy of Plk1 kinase inhibitors.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of activating a polo-like kinase-1 (Plk1) protein, the method comprising incubating a Plk1 protein in a buffer comprising (i) a divalent cation selected from the group consisting of manganese, calcium, nickel, and zinc and (ii) adenosine triphosphate (ATP), wherein the divalent cation and ATP are present in amounts sufficient to increase the kinase activity of the Plk1 protein.

2. The method of claim 1, wherein the divalent cation is manganese.

3. The method of claim 1, wherein the buffer comprises manganese chloride (MnCl₂).

4. The method of claim 3, wherein the buffer comprises at least 10 mM MnCl₂.

5. The method of claim 1, wherein the buffer comprises a detergent.

6. The method of claim 5, wherein the detergent is 3-[(3-Cholamidopropyl)dimethyl ammonio]-1-propanesulfonate (CHAPS).

7. The method of claim 6, wherein the buffer comprises at least 0.05% CHAPS.

8. The method of claim 1, wherein at least 100 μg/ml of the Plk1 protein is incubated in the buffer.

9. The method of claim 8, wherein at least 165 μg/ml of the Plk1 protein is incubated in the buffer.

10. The method of claim 1, wherein the Plk1 protein is incubated in the buffer for a period of at least one hour.

11. The method of claim 1, wherein the PLK1 protein is activated in the absence of a Plk1 substrate.

12. The method of claim 1, wherein the PLK1 protein is activated in the presence of a Plk1 substrate.

13. A method of detecting the kinase activity of a Plk1 protein, the method comprising:
providing a Plk1 protein activated by the method of claim 1;
contacting the activated Plk1 protein with a Plk1 substrate under conditions effective to permit phosphorylation of the Plk1 substrate; and
measuring phosphorylation of the Plk1 substrate,
wherein phosphorylation of the Plk1 substrate indicates kinase activity of the Plk1 protein.

14. The method of claim 13, wherein the Plk1 substrate is a polypeptide comprising full length membrane-associated tyrosine- and threonine-specific cdc-2 inhibitory kinase (Myt1) or a fragment thereof that is subject to phosphorylation by Plk1.

15. The method of claim 13, wherein the Plk1 substrate is a polypeptide comprising a fragment of Myt1 that is subject to phosphorylation by Plk1 on a serine residue that corresponds to position 426 of Myt1, on a serine residue that corresponds to position 435 of Myt1, on a serine residue that corresponds to position 469 of Myt1, or on a threonine residue that corresponds to position 495 of Myt1.

16. The method of claim 15, wherein the Plk1 substrate comprises SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:14.

17. The method of claim 13, wherein the Plk1 substrate is less than 50 amino acids in length.

18. The method of claim 13, wherein the Plk1 substrate is Myt1, cell cycle phosphatase Cdc25C, Cyclin B1, early mitotic inhibitor 1 (Emi1), anaphase-promoting complex/cyclosome 1 (APC1), anaphase-promoting complex/cyclosome subunit 3 (APC3), anaphase-promoting complex subunit 8 (APC8), nucleolar phosphoprotein B23 (B23/Nucleophosmin), breast cancer type 2 susceptibility protein homolog (BRCA2), centrosomal protein of 55 kDa (Cep55), kinesin family member 23 (KIF23/CHO1/Mklp1), Cohesin, Cohesin, golgi reassembly stacking protein 1 (GRASP65), heat shock transcription factor 1 (HSF1), Kizuna, kinesin family member 20A (KIF20A/MkIp2/Rabkinesin6), ninein-like protein (Nlp), nuclear migration protein nudC (NudC), p53, Plk1-interacting checkpoint helicase (PICH), peptidylprolyl cis/trans isomerase, NIMA-interacting 1 (Pin1), stathmin 1/oncoprotein 18 (Stathmin/Op18), translationally-controlled tumor protein homolog (TCTP), Vimentin, Weel, tumor protein p73 (p73), Bora, DNA topoisomerase II alpha, origin recognition complex 1 (Hbo1), Aurora B, Mitotic centromere-associated kinesin (MCAK), Rho-associated, coiled-coil containing protein kinase 2, MLF1 interacting protein (PBIP1), budding uninhibited by benzimidazoles 1 homolog beta (BubR1), cytoplasmic polyadenylation element-binding protein (CPEB), human phosphatase HsCdc14A, or small GTP/GDP-binding protein Ran.

19. A method of detecting the kinase activity of a Plk1 protein, the method comprising:
contacting a Plk1 protein with a Plk1 substrate under conditions effective to permit phosphorylation of the Plk1 substrate, wherein the Plk1 substrate is a polypeptide comprising full length Myt1 or a fragment of Myt1 that is subject to phosphorylation by Plk1 on a threonine residue that corresponds to position 495 of Myt1;
contacting the Plk1 substrate with a phospho-specific anti-pT495 Myt1 antibody; and
measuring binding of the antibody to the Plk1 substrate to thereby detect phosphorylation of the Plk1 substrate;
wherein phosphorylation of the Plk1 substrate indicates kinase activity of the Plk1 protein.

20. The method of claim 19, wherein, prior to contacting the Plk1 protein with the Plk1 substrate, the Plk1 protein is incubated in a buffer comprising an amount of manganese and ATP sufficient to increase the kinase activity of the Plk1 protein.

21. A method of identifying a compound that inhibits phosphorylation of a Plk1 substrate, the method comprising:
providing a Plk1 protein activated by the method of claim 1;
contacting, in the presence of a candidate compound, the activated Plk1 protein with a Plk1 substrate; and
measuring phosphorylation of the Plk1 substrate;
wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

22. A method of identifying a compound that inhibits phosphorylation of a Plk1 substrate, the method comprising:
contacting, in the presence of a candidate compound, a Plk1 protein with a Plk1 substrate, wherein the Plk1 substrate is a polypeptide comprising full length Myt1 or a fragment of Myt1 that is subject to phosphorylation by Plk1 on a threonine residue that corresponds to position 495 of Myt1;
contacting the Plk1 substrate with a phospho-specific anti-pT495 Myt1 antibody; and
measuring binding of the antibody to the Plk1 substrate to thereby detect phosphorylation of the Plk1 substrate;
wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

23. The method of claim 19, wherein the measuring occurs in a cell.

24. The method of claim 13, wherein measuring phosphorylation of the Plk1 substrate comprises:
contacting the Plk1 substrate with an antibody that (i) is conjugated to a first fluorescent agent and (ii) specifically binds to the Plk1 substrate when the Plk1 substrate is phosphorylated on a serine or threonine residue, wherein the Plk1 substrate is conjugated to a second fluorescent agent; and
detecting the occurrence of fluorescence resonance energy transfer between the first fluorescent agent and the second fluorescent agent as an indicator of phosphorylation of the Plk1 substrate.

25. The method of claim 13, wherein measuring phosphorylation of the Plk1 substrate comprises:
contacting the Plk1 substrate with an antibody that (i) is conjugated to a detection moiety and (ii) specifically binds to the Plk1 substrate when the Plk1 substrate is phosphorylated on a serine or threonine residue;
removing antibody that is not bound to the Plk1 substrate; and
detecting the detection moiety associated with the Plk1 substrate as an indicator of phosphorylation of the Plk1 substrate.

26. The method of claim 13, wherein measuring phosphorylation of the Plk1 substrate comprises passaging the Plk1 substrate through a stationary phase, wherein increased or decreased retardation of the Plk1 substrate during passage through the stationary phase indicates the phosphorylation status of the Plk1 substrate.

27. The method of claim 19, wherein the contacting occurs in a cell.

28. The method of claim 27, wherein the cell is a mammalian cell.

29. A method of assessing the ability of a compound to inhibit phosphorylation of a Plk1 substrate by a Plk1 protein in a cell, the method comprising:
providing a cell expressing a Plk1 protein and a Plk1 substrate;
incubating the cell in the presence of the compound identified by the method of claim 21; and
measuring the amount of the Plk1 substrate in the cell after incubating the cell in the presence of the compound;
wherein a difference in the amount of the Plk1 substrate in the cell after incubation with the compound as compared to the amount of the Plk1 substrate in the cell in the absence of incubation with the compound indicates that the compound inhibits phosphorylation of the Plk1 substrate by the Plk1 protein.

30. The method of claim 29, wherein the Plk1 substrate is Emi1.

31. The method of claim 30 wherein an increase in the amount of Emi1 in the cell after incubation with the compound as compared to the amount of Emi1 in the cell in the absence of incubation with the compound indicates that the compound inhibits phosphorylation of Emi1 by the Plk1 protein.

32. An isolated peptide that is less than 50 amino acids in length and comprises SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or a variant thereof, wherein the variant is a phosphorylation substrate of Plk1.

33. The peptide of claim 32, wherein the peptide comprises SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

34. The peptide of claim 33, wherein the peptide comprises a variant of SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12 in which at least one but not more than five amino acid residues are substituted, deleted, or inserted.

35. The peptide of claim 34, wherein the peptide consists of SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

36. The peptide of claim 32, wherein the peptide is phosphorylated on a threonine residue that corresponds to position 495 of Myt1.

37. The peptide of claim 36, wherein the peptide has the amino acid sequence depicted in SEQ ID NO:13.

38. An isolated antibody that specifically binds to a peptide whose amino acid sequence consists of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

39. The antibody of claim 38, wherein the antibody preferentially binds the peptide when phosphorylated on a threonine amino acid residue that corresponds to position 495 of Myt1.

40. A method of generating an immune response in a mammal, the method comprising administering to the mammal an effective amount of the peptide of claim to 31.

41. The method of claim 13, wherein the Plk1 substrate is centromere protein B (CENPB).

42. A method of detecting the kinase activity of a Plk1 protein, the method comprising:
contacting a Plk1 protein with a Plk1 substrate under conditions effective to permit phosphorylation of the Plk1 substrate, wherein the Plk1 substrate is a polypeptide comprising a CENPB protein or a fragment of a CENPB protein that is subject to phosphorylation by Plk1; and
measuring phosphorylation of the Plk1 substrate;
wherein phosphorylation of the Plk1 substrate indicates kinase activity of the Plk1 protein.

43. The method of claim 42, wherein the CENPB protein comprises the sequence of SEQ ID NO:19.

44. The method of claim 42, wherein the Plk1 substrate is a polypeptide comprising a fragment of a CENPB protein that is subject to phosphorylation by Plk1 on a serine residue that corresponds to position 43 of CENPB, on a serine residue that corresponds to position 156 of CENPB, on a threonine residue that corresponds to position 169 of CENPB, on a serine residue that corresponds to position 307 of CENPB, or on a threonine residue that corresponds to position 396 of CENPB.

45. The method of claim 44, wherein the Plk1 substrate comprises SEQ ID NO:21, SEQ ID NO:22, SEQ ID NO:23, SEQ ID NO:24, or SEQ ID NO:25.

46. The method of claim 42, wherein the Plk1 substrate is less than 50 amino acids in length.

47. A method of identifying a compound that inhibits phosphorylation of a Plk1 substrate, the method comprising:
contacting, in the presence of a candidate compound, a Plk1 protein with a Plk1 substrate, wherein the Plk1 substrate is a polypeptide comprising a CENPB protein or a fragment of a CENPB protein that is subject to phosphorylation by Plk1; and
measuring phosphorylation of the Plk1 substrate;
wherein decreased phosphorylation of the Plk1 substrate in the presence of the candidate compound as compared to phosphorylation of the Plk1 substrate that occurs in the absence of the candidate compound indicates that the candidate compound inhibits phosphorylation of the Plk1 substrate by the Plk1 protein.

48. The method of claim 42, wherein measuring phosphorylation of the Plk1 substrate comprises:
contacting the Plk1 substrate with a phospho-specific anti-CENPB antibody; and
measuring binding of the antibody to the Plk1 substrate to thereby detect phosphorylation of the Plk1 substrate.

49. The method of claim 48, wherein the phospho-specific anti-CENPB protein antibody specifically recognizes CENPB at an epitope comprising a phosphorylated serine residue that corresponds to position 43 of CENPB, a phosphorylated serine residue that corresponds to position 156 of CENPB, a phosphorylated threonine residue that corresponds to position 169 of CENPB, a phosphorylated serine residue that corresponds to position 307 of CENPB, or a phosphorylated threonine residue that corresponds to position 396 of CENPB.

50. The method of claim 42, wherein measuring phosphorylation of the Plk1 substrate comprises:
contacting the Plk1 substrate with an antibody that (i) is conjugated to a detection moiety and (ii) specifically binds to the Plk1 substrate when the CENPB protein is phosphorylated on a serine or threonine residue; and
detecting the detection moiety associated with the Plk1 substrate as an indicator of phosphorylation of the Plk1 substrate.

51. The method of claim 42, wherein measuring phosphorylation of the Plk1 substrate comprises passaging the Plk1 substrate through a stationary phase, wherein an increased or decreased retardation of the Plk1 substrate during passage through the stationary phase indicates the phosphorylation status of the Plk1 substrate.

52. A method for identifying a compound that inhibits an interaction between a Plk1 protein and a Plk1 substrate, the method comprising:
contacting, in the presence of a candidate compound, a Plk1 protein with a Plk1 substrate, wherein the Plk1 substrate is a polypeptide comprising a CENPB protein or a fragment of a CENPB protein that binds to Plk1; and
measuring binding of the Plk1 protein to the Plk1 substrate;
wherein decreased binding of the Plk1 protein to the Plk1 substrate in the presence of the candidate compound as compared to binding of the Plk1 protein to the Plk1 substrate that occurs in the absence of the candidate compound indicates that the candidate compound inhibits an interaction between the Plk1 protein and the Plk1 substrate.

53. The method of claim 42, wherein the measuring occurs in a cell.

54. A method of inhibiting phosphorylation of a CENPB protein by a Plk1 protein, the method comprising administering to a subject an effective amount of a compound that inhibits phosphorylation of a CENPB protein by a Plk1 protein.

55. The method of claim 54, wherein the compound is a polypeptide comprising a CENPB protein or a fragment of a CENPB protein that is subject to phosphorylation by Plk1.

56. A method of inhibiting an interaction between a Plk1 protein and a CENPB protein, the method comprising administering to a subject an effective amount of a compound that inhibits an interaction between a Plk1 protein and a CENPB protein.

57. The method of claim 56, wherein the compound is a polypeptide comprising a CENPB protein or a fragment of a CENPB protein that binds to Plk1.

58. The method of claim 54, wherein the subject is a mammal.

59. The method of claim 58, wherein the mammal is a human.

60. The method of claim 54, wherein the subject has a cancer.

61. The method of claim 60, further comprising determining if one or more cells of the subject's cancer express a Plk1 protein, a CENPB protein, or a Plk1 protein and a CENPB protein.

62. A method for evaluating the efficacy of an anti-Plk1 agent, the method comprising:
providing a biological sample obtained from a subject to whom an anti-Plk1 agent has been administered; and
detecting phosphorylation of a CENPB protein in the biological sample;wherein a decreased level of phosphorylation of the CENPB protein as compared to the level of phosphorylation in a biological sample taken from another subject or from the subject prior to administration of the anti-Plk1 agent indicates that the anti-Plk1 therapy is effective.

63. The method of claim 62, wherein the anti-Plk1 agent inhibits Plk1 kinase activity.

64. The method of claim 62, wherein the anti-Plk1 agent inhibits Plk1 expression.

65. The method of claim 62, wherein the anti-Plk1 agent is scytonemin, ON01910, or BI 2536.

66. An isolated peptide that is less than 50 amino acids in length and comprises a fragment of a CENPB protein that is subject to phosphorylation by Plk1 on a serine residue that corresponds to position 43 of CENPB, on a serine residue that corresponds to position 156 of CENPB, on a threonine residue that corresponds to position 169 of CENPB, on a serine residue that corresponds to position 307 of CENPB, or on a threonine residue that corresponds to position 396 of CENPB.

67. An isolated peptide that is less than 50 amino acids in length and comprises SEQ ID NO:21, SEQ ID NO:22, SEQ ID NO:23, SEQ ID NO:24, or SEQ ID NO:25, or a variant thereof, wherein the variant is a phosphorylation substrate of Plk1.

68. The peptide of claim 67, wherein the peptide comprises SEQ ID NO:21, SEQ ID NO:22, SEQ ID NO:23, SEQ ID NO:24, or SEQ ID NO:25.

69. The peptide of claim 67, wherein the peptide comprises a variant of SEQ ID NO:21, SEQ ID NO:22, SEQ ID NO:23, SEQ ID NO:24, or SEQ ID NO:25 in which at least one but not more than five amino acid residues are substituted, deleted, or inserted.

70. The peptide of claim 67, wherein the peptide consists of SEQ ID NO:21, SEQ ID NO:22, SEQ ID NO:23, SEQ ID NO:24, or SEQ ID NO:25.

71. The peptide of claim 66, wherein the peptide is phosphorylated on a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB.

72. An isolated antibody that specifically binds to the peptide of claim 66.

73. The antibody of claim 72, wherein the antibody preferentially binds the peptide when phosphorylated on a serine residue that corresponds to position 43 of CENPB, a serine residue that corresponds to position 156 of CENPB, a threonine residue that corresponds to position 169 of CENPB, a serine residue that corresponds to position 307 of CENPB, or a threonine residue that corresponds to position 396 of CENPB.

74. A method of generating an immune response in a mammal, the method comprising administering to the mammal an effective amount of the peptide of claim 66.

75. A method for generating a compound that inhibits the interaction between a Plk1 protein and a CENPB protein, the method comprising:
providing a three-dimensional structure of a molecule or a molecular complex comprising: (a) a Plk1 protein or a CENPB-binding fragment thereof; (b) a CENPB protein or a Plk1-binding fragment thereof; or (c) a molecular complex comprising (a) and (b);
designing, based on the three-dimensional structure, a compound comprising a region that inhibits the interaction between a Plk1 protein and a CENPB protein; and
producing the compound.


SEQUENCE LISTING (25 sequences)

SEQ ID NO: 1 - protein, 603 aa, homo sapiens [sequence omitted]

SEQ ID NO: 2 - protein, 499 aa, homo sapiens
MLERPPALAMPMPTEGTPPPLSGTPIPVPAYFRHAEPGFSLKRPRGLSRSLPPPPPAKGSIPISRLFPPRTPGWHQLQPRRVSFRGEASETLQSPGYDPSRPESFFQQSFQRLSRLGHGSYGEVFKVRSKEDGRLYAVKRSMSPFRGPKDRARKLAEVGSHEKVGQHPCCVRLEQAWEEGGILYLQTELCGPSLQQHCEAWGASLPEAQVWGYLRDTLLALAHLHSQGLVHLDVKPANIFLGPRGRCKLGDFGLLVELGTAGAGEVQEGDPRYMAPELLQGSYGTAADVFSLGLTILEVACNMELPHGGEGWQQLRQGYLPPEFTAGLSSELRSVLVMMLEPDPKLRATAEALLALPVLRQPRAWGVLWCMAAEALSRGWALWQALLALLCWLWHGLAHPASWLQPLGPPATPPGSPPCSLLLDSSLSSNWDDDSLGPSLSPEAVLARTVGSTSTPRSRCTPRDALDLSDINSEPPRGSFPSFEPRNLLSLFEDTLDPT

SEQ ID NO: 3 - protein, 473 aa, homo sapiens
MSTELFSSTREEGSSGSGPSFRSNQRKMLNLLLERDTSFTVCPDVPRTPVGKFLGDSANLSILSGGTPKRCLDLSNLSSGEITATQLTTSADLDETGHLDSSGLQEVHLAGMNHDQHLMKCSPAQLLCSTPNGLDRGHRKRDAMCSSSANKENDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 4 - protein, 15 aa, Artificial
  Plk1 Substrate
PRNLLSLFEDTLDPT

SEQ ID NO: 5 - protein, 11 aa, Artificial
  Plk1 substrate
NLLSMFEDTLD

SEQ ID NO: 6 - protein, 15 aa, Artificial
  Plk1 substrate
PRNLLSMFEDTLDPT

SEQ ID NO: 7 - protein, 11 aa, Artificial
  Plk1 substrate
NLLSLFEDTLD

SEQ ID NO: 8 - protein, 15 aa, Artificial
  Plk1 substrate
FEPRNLLSLFEDTLD

SEQ ID NO: 9 - protein, 19 aa, Artificial
  Plk1 substrate
SFPSFEPRNLLSLFEDTLD

SEQ ID NO: 10 - protein, 23 aa, Artificial
  Plk1 substrate
PPRGSFPSFEPRNLLSLFEDTLD

SEQ ID NO: 11 - protein, 21 aa, Artificial
  Plk1 ubstrate
SFPSFEPRNLLSLFEDTLDPT

SEQ ID NO: 12 - protein, 14 aa, Artificial
  Plk1 substrate
CNLLSLFEDTLDPT

SEQ ID NO: 13 - protein, 14 aa, Artificial
  Plk1 substrate
CNLLSLFEDTLDPT

SEQ ID NO: 14 - protein, 499 aa, homo sapiens [sequence omitted]

SEQ ID NO: 15 - DNA, 19 bp, Artificial
  SiRNA
caaccaaagtcgaatatga

SEQ ID NO: 16 - DNA, 20 bp, Artificial
  SiRNA
caagaagaatgaatacaggt

SEQ ID NO: 17 - DNA, 20 bp, Artificial
  SiRNA
gaagatgtccatggaaatat

SEQ ID NO: 18 - DNA, 18 bp, Artificial
  SiRNA
caacacgcctcatcctct

SEQ ID NO: 19 - protein, 599 aa, Homo sapiens
MGPKRRQLTFREKSRIIQEVEENPDLRKGEIARRFNIPPSTLSTILKNKRAILASERKYGVASTCRKTNKLSPYDKLEGLLIAWFQQIRAAGLPVKGIILKEKALRIAEELGMDDFTASNGWLDRFRRRHGVVSCSGVARARARNAAPRTPAAPASPAAVPSEGSGGSTTGWRAREEQPPSVAEGYASQDVFSATETSLWYDFLPDQAAGLCGGDGRPRQATQRLSVLLCANADGSEKLPPLVAGKSAKPRAGQAGLPCDYTANSKGGVTTQALAKYLKALDTRMAAESRRVLLLAGRLAAQSLDTSGLRHVQLAFFPPGTVHPLERGVVQQVKGHYRQAMLLKAMAALEGQDPSGLQLGLTEALHFVAAAWQAVEPSDIAACFREAGFGGGPNATITTSLKSEGEEEEEEEEEEEEEEGEGEEEEEEGEEEEEEGGEGEELGEEEEVEEEGDVDSDEEEEEDEESSSEGLEAEDWAQGVVEAGGSFGAYGAQEEAQCPTLHFLEGGEDSDSDSEEEDDEEEDDEDEDDDDDEEDGDEVPVPSFGEAMAYFAMVKRYLTSFPIDDRVQSHILHLEHDLVHVTRKNHARQAGVRGLGHQS

SEQ ID NO: 20 - protein, 4 aa, Artificial
  Domain
DTSG

SEQ ID NO: 21 - protein, 12 aa, Artificial
  Plk1 substrate
NIPPSTLSTILK

SEQ ID NO: 22 - protein, 24 aa, Artificial
  Plk1 substrate
TPAAPASPAAVPSEGSGGSTTGWR

SEQ ID NO: 23 - protein, 12 aa, Artificial
  Plk1 substrate
LAAQSLDTSGLR

SEQ ID NO: 24 - protein, 17 aa, Artificial
  Plk1 substrate
EAGFGGGPNATITTSLK

SEQ ID NO: 25 - protein, 16 aa, Artificial
  Plk1 substrate
SEGSGGSTTGWRAREE